SEQUENCE LISTING (62 sequences)

SEQ ID NO: 1 - protein, 33 aa, Artificial Sequence
  SYNTHETIC
VXAKXLAXPGAIXGVLMALXQNAAVAANAAIRA

SEQ ID NO: 2 - protein, 17 aa, Artificial Sequence
  SYNTHETIC
SNCTSKGSPASCCSCCC

SEQ ID NO: 3 - protein, 21 aa, Artificial Sequence
  SYNTHETIC
STSCSSTSTCSSTTSTTSCSA

SEQ ID NO: 4 - protein, 30 aa, Artificial Sequence
  SYNTHETIC
NGCNCAGCVGCGLNCMSGGWKCATCGSACC

SEQ ID NO: 5 - protein, 38 aa, Artificial Sequence
  SYNTHETIC
SVIIIFGYTEDGEEILTEDSLNSEAAGSGSLSTSSSCC

SEQ ID NO: 6 - protein, 28 aa, Artificial Sequence
  SYNTHETIC
SVSSLLSAACPVSSFSSVSNSVAEVSAE

SEQ ID NO: 7 - protein, 21 aa, Artificial Sequence
  SYNTHETIC
STSCSSTSTCSSTTSTTSCSA

SEQ ID NO: 8 - protein, 30 aa, Artificial Sequence; identical to SEQ ID NO: 4
  SYNTHETIC

SEQ ID NO: 9 - protein, 21 aa, Artificial Sequence
  SYNTHETIC
STSCSSTSTCSSTTSTTSCSA

SEQ ID NO: 10 - protein, 23 aa, Artificial Sequence
  SYNTHETIC
SWGSCSCQGSSSCAQPQVETPTA

SEQ ID NO: 11 - protein, 13 aa, Artificial Sequence
  SYNTHETIC
SSCCCCCPCCSCT

SEQ ID NO: 12 - protein, 17 aa, Artificial Sequence
  SYNTHETIC
SNCTSKGSPASCCSCCC

SEQ ID NO: 13 - protein, 21 aa, Artificial Sequence
  SYNTHETIC
STSCSSTSTCSSTTSTTSCSA

SEQ ID NO: 14 - protein, 30 aa, Artificial Sequence; identical to SEQ ID NO: 4
  SYNTHETIC

SEQ ID NO: 15 - protein, 38 aa, Artificial Sequence
  SYNTHETIC
SVIIIFGYTEDGEEILTEDSLNSEAAGCGSLSTSSSCC

SEQ ID NO: 16 - protein, 28 aa, Artificial Sequence
  SYNTHETIC
SVSSLLSAACPVSSFSSVSNSVAEVSAE

SEQ ID NO: 17 - protein, 16 aa, Artificial Sequence
  SYNTHETIC
STTTTGCTTSSCSSSS

SEQ ID NO: 18 - protein, 16 aa, Artificial Sequence
  SYNTHETIC
SCVGTACACSSTSSSS

SEQ ID NO: 19 - protein, 21 aa, Artificial Sequence
  SYNTHETIC
STSCSSTSTCSSTTSTTSCSA

SEQ ID NO: 20 - protein, 21 aa, Artificial Sequence
  SYNTHETIC
STSCSSTSTCSSTTSTTSCSA

SEQ ID NO: 21 - protein, 30 aa, Artificial Sequence; identical to SEQ ID NO: 4
  SYNTHETIC

SEQ ID NO: 22 - protein, 20 aa, Artificial Sequence
  SYNTHETIC
TFLQALWPFLVVLSCRRSTS

SEQ ID NO: 23 - protein, 21 aa, Artificial Sequence
  SYNTHETIC
STSCSSTSTCSSTTSTTSCSA

SEQ ID NO: 24 - protein, 30 aa, Artificial Sequence; identical to SEQ ID NO: 4
  SYNTHETIC

SEQ ID NO: 25 - protein, 45 aa, Artificial Sequence
  SYNTHETIC
CGSYCECYCPSPTDDSGRFQPCGDLMGQQCPERCCLVCVDCQCEE

SEQ ID NO: 26 - protein, 28 aa, Artificial Sequence
  SYNTHETIC
TFSTTSTASTASCPASSAGSVMTANCTG

SEQ ID NO: 27 - protein, 27 aa, Artificial Sequence
  SYNTHETIC
WFSAASASTASCPGSSAATVGTASTFG

SEQ ID NO: 28 - protein, 28 aa, Artificial Sequence
  SYNTHETIC
SFSSATSASTASCPATSASSLTSASTIG

SEQ ID NO: 29 - protein, 29 aa, Artificial Sequence
  SYNTHETIC
SSVSTAGSVGTASCPATSASSFYSFSSYT

SEQ ID NO: 30 - protein, 28 aa, Artificial Sequence
  SYNTHETIC
GAAAGGAGGAPQQQAPGGGGCCGGGCCG

SEQ ID NO: 31 - protein, 21 aa, Artificial Sequence
  SYNTHETIC
STSCSSTSTCSSTTSTTSCSA

SEQ ID NO: 32 - protein, 30 aa, Artificial Sequence
  SYNTHETIC
NGCNSAGCVGSGLNCMSGGWKCATSGSACC

SEQ ID NO: 33 - protein, 30 aa, Artificial Sequence; identical to SEQ ID NO: 4
  SYNTHETIC

SEQ ID NO: 34 - protein, 25 aa, Artificial Sequence
  SYNTHETIC
LKIGHHMRTVFEQYVEEWQGSSTGN

SEQ ID NO: 35 - protein, 18 aa, Artificial Sequence
  SYNTHETIC
SNCTSTGTPASCCSCCCC

SEQ ID NO: 36 - protein, 21 aa, Artificial Sequence
  SYNTHETIC
STSCSSTSTCSSTTSTTSCSA

SEQ ID NO: 37 - protein, 29 aa, Artificial Sequence
  SYNTHETIC
GCNCAGCVGCGLNCMSGGWKCATCGSACC

SEQ ID NO: 38 - protein, 21 aa, Artificial Sequence
  SYNTHETIC
STSCSSTSTCSSTTSTTSCSA

SEQ ID NO: 39 - protein, 30 aa, Artificial Sequence; identical to SEQ ID NO: 4
  SYNTHETIC

SEQ ID NO: 40 - protein, 21 aa, Artificial Sequence
  SYNTHETIC
STSCSSTSTCSSTTSTTSCSA

SEQ ID NO: 41 - protein, 23 aa, Artificial Sequence
  SYNTHETIC
SWGSCSCQGSSSCAQPQVETPTA

SEQ ID NO: 42 - protein, 13 aa, Artificial Sequence
  SYNTHETIC
SSCCCCCPCCSCT

SEQ ID NO: 43 - protein, 22 aa, Artificial Sequence
  SYNTHETIC
SSTSCGSSSCSSSTTSTTSTCA

SEQ ID NO: 44 - protein, 21 aa, Artificial Sequence
  SYNTHETIC
STSCTSCSTTSSTTSCTSCCA

SEQ ID NO: 45 - protein, 24 aa, Artificial Sequence
  SYNTHETIC
SWGSCSCQASSSCAQPHVTTPEVL

SEQ ID NO: 46 - protein, 21 aa, Artificial Sequence
  SYNTHETIC
STSCSSTSTCSSTTSTTSCSA

SEQ ID NO: 47 - protein, 30 aa, Artificial Sequence; identical to SEQ ID NO: 4
  SYNTHETIC

SEQ ID NO: 48 - protein, 21 aa, Artificial Sequence
  SYNTHETIC
TFLQALWPFLVVLSCRRSTST

SEQ ID NO: 49 - protein, 21 aa, Artificial Sequence
  SYNTHETIC
STSCSSTSTCSSTTSTTSCSA

SEQ ID NO: 50 - protein, 30 aa, Artificial Sequence; identical to SEQ ID NO: 4
  SYNTHETIC

SEQ ID NO: 51 - protein, 21 aa, Artificial Sequence
  SYNTHETIC
STSCSSTSTCSSTTSTTSCSA

SEQ ID NO: 52 - protein, 30 aa, Artificial Sequence; identical to SEQ ID NO: 4
  SYNTHETIC

SEQ ID NO: 53 - protein, 21 aa, Artificial Sequence
  SYNTHETIC
STSCSSTSTCSSTTSTTSCSA

SEQ ID NO: 54 - protein, 30 aa, Artificial Sequence
  SYNTHETIC
NGCNCAGCVGCGLNCMSGGWICATCGSACC

SEQ ID NO: 55 - protein, 45 aa, Artificial Sequence; identical to SEQ ID NO: 25
  SYNTHETIC

SEQ ID NO: 56 - protein, 21 aa, Artificial Sequence
  SYNTHETIC
STSCSSTSTCSSTTSTTSCSA

SEQ ID NO: 57 - protein, 30 aa, Artificial Sequence; identical to SEQ ID NO: 4
  SYNTHETIC

SEQ ID NO: 58 - protein, 21 aa, Artificial Sequence
  SYNTHETIC
STSCSSTSTCSSTTSTTSCSA

SEQ ID NO: 59 - protein, 30 aa, Artificial Sequence; identical to SEQ ID NO: 4
  SYNTHETIC

SEQ ID NO: 60 - protein, 21 aa, Artificial Sequence
  SYNTHETIC
STSCSSTSTCSSTTSTTSCSA

SEQ ID NO: 61 - protein, 28 aa, Artificial Sequence
  SYNTHETIC
VSVSSLLSAACPVSSFSSVSNSVAEVAA

SEQ ID NO: 62 - protein, 21 aa, Artificial Sequence
  SYNTHETIC
STSCSSTSTCSSTTSTTSCSA

(19) United States
(12) Patent Application Publication
(10) Pub. No.: US 2017/0227545 A1
(43) Pub. Date: Aug. 10, 2017

(54) REACTIVITY-BASED SCREENING FOR NATURAL PRODUCT DISCOVERY

(71) Applicant: THE BOARD OF TRUSTEES OF THE UNIVERSITY OF ILLINOIS, Urbana, IL (US)

(72) Inventors: Douglas A. Mitchell, Urbana, IL (US); Jonathan Tietz, Champaign, IL (US)

(21) Appl. No.: 15/317,328

(22) PCT Filed: Jun. 10, 2015
(86) PCT No.: PCT/US2015/035215

§ 371 Date: Dec. 8, 2016

Related U.S. Application Data
(60) Provisional application No. 62/010,280, filed on Jun. 10, 2014.

(51) Int. Cl. G01N 33/68; G06F 19/12; C07K 7/56

(52) U.S. Cl. CPC G01N 33/6848; C07K 7/56; G06F 19/12; G01N 2333/36; G01N 2333/195; G01N 2560/00; G06F 19/706

(57) ABSTRACT

A method of identifying a natural product comprising NP—[X]ₙ is provided. The method includes several steps. The first step includes selecting an organism having a biosynthetic pathway for producing the natural product comprising NP—[X]ₙ using a bioinformatics algorithm. The second step includes preparing a sample suspected to contain NP—[X]ₙ including a complex cellular metabolite mixture from an organism. The third step includes reacting the sample suspected to contain NP—[X]ₙ with reactivity probe Y according to Scheme I: Scheme I. NP—[X]ₙ represents a natural product NP having a chemical moiety X that is susceptible to chemical modification by reactivity probe Y to form at least one product adduct NP—[X]ₙ₋ₘ [Z]ₙ in which chemical moiety X reacts with reactivity probe Y to form adduct Z, wherein n ranges from 1 to about 10 and m is at least 1 and m≦n. The fourth step includes optionally dereplicating the product collection of at least one known labeled metabolite to provide a depleted product collection including at least one unknown labeled metabolite. The fifth step includes determining the structure of the at least one unknown labeled metabolite, thereby identifying the natural product comprising NP—[X]ₙ.

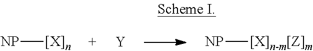

### CROSS-REFERENCE TO RELATED APPLICATIONS

This application claims benefit of priority under 35 U.S.C. 119 to U.S. provisional patent application serial numbers 62/010,280, filed Jun. 10, 2014, and entitled “REACTIVITY-BASED SCREENING FOR NATURAL PRODUCT DISCOVERY,” the contents of which are herein incorporated by reference in its entirety.

### STATEMENT REGARDING FEDERALLY SPONSORED RESEARCH OR DEVELOPMENT

This invention was made with government support under DP20D008463 awarded by the National Institutes of Health. The government has certain rights in the invention.

### FIELD OF THE INVENTION

This invention pertains to methods for identifying natural products. In particular, the methods are directed to identifying natural products from organisms using a combination of bioinformatics-guided organism prioritization and reactivity-based screening. The methods are robust by eliminating known natural products before conducting detailed physicochemical characterization of candidate natural products.

### BACKGROUND OF THE INVENTION

Bacteria have historically been a rich reservoir of architecturally complex natural products exhibiting antibiotic activity (Newman, D. J., Cragg, G. M. (2012) “Natural products as sources of new drugs over the 30 years from 1981 to 2010,” J. Nat. Prod. 75, 311-335). However, the traditional approach to natural product discovery—bioassay-guided isolation of compounds from extracts—is limited by high rates of compound rediscovery (Lewis, K. (2013) “Platforms for antibiotic discovery,” Nat. Rev. Drug Discov. 12, 371-387)). As such, the potential value of novel natural products to advance the treatment of disease, and in particular to address the issue of antibiotic resistance (Fischbach, M. A., Walsh, C. T. (2009) “Antibiotics for emerging pathogens,” Science. 325, 1089-1093), warrants the development of alternative strategies to discover novel compounds. The advent of widely available genome sequences makes bioinformatics-driven methods increasingly appealing, since the enzymatic machinery responsible for natural product biosynthesis can be readily identified (Deane, C. D., Mitchell, D. A. (2014) “Lessons learned from the transformation of natural product discovery to a genome-driven endeavor,” J. Ind. Microbiol. Biotechnol. 41, 315-31; Velasquez, J. E., van der Donk, W. A. (2011) “Genome mining for ribosomally synthesized natural products,” Curr. Opin. Chem. Biol. 15, 11-21). Consequently, a number of strategies have emerged that aid in connecting biosynthetic gene clusters to their products, including selective enzymatic derivatization (Gao, J. et al. (2014) “Use of a Phosphonate Methyltransferase in the Identification of the Fosfazinomycin Biosynthetic Gene Cluster,” Angew. Chem., Int. Ed. 126, 1358-1361), chemoselective enrichment (Odendaal, A. Y. et al. (2011) “Chemoselective enrichment for natural products discovery,” Chem. Sci. 2, 760-764), mass spectrometry-based network analysis (Nguyen, D. D. et al. (2013) “MS/MS networking guided analysis of molecule and gene cluster families,” Proc. Natl. Acad. Sci. U.S.A. 110, E2611-E2620), and PCR prioritization (Xie, P. et al. (2014) “Biosynthetic potential-based strain prioritization for natural product discovery: a showcase for diterpenoid-producing actinomycetes,” J Nat Prod. 77, 377-387) among others.

Many classes of dehydrated amino acid (DHAA)-bearing natural products are ribosomally produced, rendering them ideal for genome-guided discovery. The availability of genome sequences has revealed a tremendous biosynthetic capability among diverse microbial species (Challis, G. L. (2008) “Genome mining for novel natural product discovery,” J. Med. Chem. 51, 2618-2628). It has become apparent that even well-characterized bacteria harbor the potential to produce an abundance of yet-uncharacterized natural products (Bentley, S. D. et al. (2002) “Complete genome sequence of the model actinomycete Streptomyces coelicolor A3(2),” Nature. 417, 141-147). To overcome the burden of rediscovery (Watve, M. G. et al. (2001) “How many antibiotics are produced by the genus Streptomyces?,” Arch Microbiol. 176, 386-390), bioinformatics can be used to preselect bacterial strains for screening to only include the organisms with the theoretical capacity to produce a particular type of natural product (Xie, P. et al. (2014)). However, even with the bioinformatics identification of promising biosynthetic gene clusters, the detection and isolation of the resultant natural products often proves to be difficult given that the products of most biosynthetic pathways are present in extremely low quantities (if present at all) during laboratory cultivation (Scherlach, K., Hertweck, C. (2009) “Triggering cryptic natural product biosynthesis in microorganisms,” Org Biomol Chem. 7, 1753-1760).

Thus, there is a need for facile methods for identifying novel natural products while avoiding the problems associated with compound rediscovery.

### BRIEF SUMMARY OF THE INVENTION

In a first aspect, a method of identifying a natural product comprising NP—[X]ₙ is disclosed. The method includes several steps. The first step includes selecting an organism having a biosynthetic pathway for producing the natural product comprising NP—[X]ₙ using a bioinformatics algorithm. The second step includes preparing a sample suspected to contain NP—[X]ₙ including a complex cellular metabolite mixture from an organism. The third step includes reacting the sample suspected to contain NP—[X]ₙ with reactivity probe Y according to Scheme I:

NP—[X]ₙ+Y→NP—[X]ₙ₋ₘ[Z]ₘ Scheme I.

NP—[X]ₙ represents a natural product NP having a chemical moiety X that is susceptible to chemical modification by reactivity probe Y to form at least one product adduct NP—[X]ₙ₋ₘ[Z]ₘ in which chemical moiety X reacts with reactivity probe Y to form adduct Z, wherein n ranges from 1 to about 10 and m is at least 1 and m≦n. The fourth step includes optionally dereplicating the product collection of at least one known labeled metabolite to provide a depleted product collection including at least one unknown labeled metabolite. The fifth step includes determining the structure of the at least one unknown labeled metabolite, thereby identifying the natural product comprising NP—[X]ₙ.

In a second aspect, a natural product comprising NP—[X]ₙ identified with the foregoing disclosed method presented herein is provided.

In a third aspect, a composition is disclosed including cyclothiazomycin C having the structure of Formula (I):

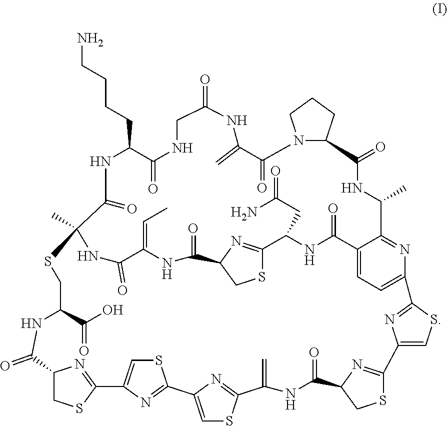 [SMILES: C=C1NC(=O)CNC(=O)[C@H](CCCCN)NC(=O)[C@@]2(C)NC(=O)/C(=C/C)NC(=O)[C@@H]3CSC(=N3)[C@H](CC(N)=O)NC(=O)c3ccc(nc3[C@@H](C)NC(=O)[C@@H]3CCCN3C1=O)-c1nc(cs1)C1=N[C@@H](CS1)C(=O)NC(=C)c1nc(cs1)-c1nc(cs1)C1=N[C@H](CS1)C(=O)N[C@H](C(=O)O)CS2]

In a fourth aspect, a method of identifying a natural product comprising NP—[X]ₙ is disclosed. The method includes several steps. The first step includes preparing a sample suspected to contain NP—[X]ₙ including a complex cellular metabolite mixture from an organism. The second step includes reacting the sample suspected to contain NP—[X]ₙ with reactivity probe Y according to Scheme I:

NP—[X]ₙ+Y→NP—[X]ₙ₋ₘ[Z]ₘ Scheme I.

NP—[X]ₙ represents a natural product NP having a chemical moiety X that is susceptible to chemical modification by reactivity probe Y to form at least one product adduct NP—[X]ₙ₋ₘ[Z]ₘ in which chemical moiety X reacts with reactivity probe Y to form adduct Z, wherein n ranges from 1 to about 10 and m is at least 1 and m≦n. The third step includes optionally dereplicating the product collection of at least one known labeled metabolite to provide a depleted product collection including at least one unknown labeled metabolite. The fourth step includes determining the structure of the at least one unknown labeled metabolite, thereby identifying the natural product comprising NP—[X]ₙ.

In a fifth aspect, a natural product comprising NP—[X]ₙ identified with the foregoing disclosed method presented herein is provided.

### BRIEF DESCRIPTION OF THE FIGURES

FIG. 1A depicts a strategy for natural product discovery by bioinformatics prioritization and nucleophilic 1,4-addition chemistry, wherein the reaction scheme for the thiol (DTT/base) labeling method with 1,4-addition sites is indicated with yellow circles.

FIG. 1B depicts a work flow for the bioinformatics-based strain prioritization, subsequent DTT-labeling, and MS screening (reactivity-based screening). (1) Prediction of DHAA-containing thiopeptide biosynthetic gene clusters from 400 in-house sequenced genomes (all from the USDA ARS Actinobacteria collection, which totals ˜9000 unique strains). More information on strain prioritization is given in FIG. 7. (2) DHAAs on exported bacterial metabolites that are reactive towards nucleophilic 1,4-additions (by DTT/base) are identified by differential mass spectrometry. (3) Compound isolation and characterization after dereplication. Compounds are dereplicated, taking only potentially novel compounds through the time-consuming characterization steps. Of the 400 sequenced genomes, 130 strains were prioritized, 23 strains were screened, 1 compound was rapidly dereplicated, and 1 compound was predicted to be novel and thus further characterized.

FIG. 2 depicts an exemplary listing of reactions and applications of Scheme I.

FIG. 3 depicts representative natural products bearing DHAAs. Structures of example molecules that contain DHAAs suitable for nucleophilic addition are shown. The sites of potential nucleophilic reactivity (i.e. the DHAA alkenes, often in the form of an α,β-unsaturated carbonyl) are indicated with yellow circles. LAP, linear azol(in)e-containing peptide.

FIG. 4A depicts the structure of thiostrepton with DHAAs suitable for nucleophilic addition highlighted with yellow circles.

FIG. 4B depicts an exemplary MALDI-TOF MS of thiostrepton labeling performed in the context of an organic, cell-surface extract of Streptomyces azureus ATCC 14921. The black spectrum (top) is an unreacted control while the red spectrum (bottom) resulted from DTT-labeling. Thiostrepton was visibly labeled by 1-5 DTT moieties, with the 4 DTT adduct being the majority product.

FIG. 5 depicts an exemplary base-dependence of the DTT-labeling reaction. MALDI-TOF MS of pure (commercially-obtained) thiostrepton reacted with DTT in the presence of diisopropylethylamine (DIPEA) (top), or no base (bottom). Thiostrepton was visibly labeled with 1-5 DTT moieties. * denotes peaks not corresponding to DTT labeling.

FIG. 6A depicts the structure of geobacillin I.

FIG. 6B depicts an exemplary Nucleophilic labeling with DTT of geobacillin I within the context of the organic extract of Geobacillus sp. M10EXG. Mass spectra of crude unlabeled extract (black spectrum, top) and DTT-labeled material (red spectrum, bottom) are shown. Extent of labeling with DTT is indicated on the bottom spectrum (2 DTT adducts are clearly observed, with the third being a very low intensity ion).

FIG. 7A depicts an exemplary bioinformatics prioritization schematic. (1) A list is populated with strains encoding a thiazole/oxazole-modified microcin (TOMM) cyclodehydratase “YcaO” necessary for the heterocyclization of select Cys, Ser and Thr residues. (2) The list of strains is then trimmed to only contain strains that also harbor a “lantibiotic” dehydratase in close proximity (within 10 open reading frames on either side) to the YcaO protein. (3) TOMM-like precursor peptides from the trimmed list are then identified, and the mass of the final natural product is predicted for use in the dereplication process. (4) If strains make it through steps 1-3, reactivity-based screening with DTT is utilized to identify natural products of interest.

FIG. 7B depicts the predicted core regions of the precursor peptides identified in the 23 strains prioritized and screened using the DTT labeling method. Highlighted in red are the precursor peptides predicted from WC-3908 (the producer of cyclothiazomycin C) and WC-3480 (the producer of grisemycin).

FIG. 8 depicts exemplary Mass spectra of strains screened by the DTT labeling method. Mass spectrometry data (m/z 900-4200 Da) is shown for all strains screened except Streptomyces griseus subsp. griseus and WC-3908 (shown as FIGS. 9 and 10, respectively). The mass spectra of the unreacted organic cell-surface extracts are shown in black with the corresponding DTT-reacted extracts in red. Each spectrum is labeled according to the strain designation (NRRL identifier) and whether or not DTT/DIPEA was added. NRRL, Northern Regional Research Laboratory collection, which is curated by the Agricultural Research Service under the supervision of the U.S. Department of Agriculture (USDA/ARS).

FIG. 9A depicts the structure of grisemycin.

FIG. 9B depicts an exemplary MALDI-TOF MS analysis of unreacted grisemycin (black spectrum, top) and DTT-labeled grisemycin (red spectrum, bottom) from an organic, cell-surface extract showing 1-2 DTT adducts.

FIG. 9C depicts an exemplary MS/MS analysis of grisemycin with the discerned sequence tag listed above the spectrum.

FIG. 10A depicts an exemplary MALDI-TOF MS analysis showing spectra of unreacted (black spectrum, top) and DTT-labeled (red spectrum, bottom) extracts of WC-3908, the producer of cyclothiazomycin C. *, peaks do not correspond to DTT-labeled cyclothiazomycin C.

FIG. 10B depicts the conserved open-reading frames from each of the three cyclothiazomycin gene clusters (precise cluster boundaries are not yet established). Genes are color-coded with proposed functions given in the legend. The strain used for the comparison of cyclothiazomycin A is Streptomyces hygroscopicus subsp. jinggangensis 5008 and cyclothiazomycin B is Streptomyces mobaraensis.

FIG. 10C depicts precursor peptide sequences of cyclothiazomycins A, B, and C. Highlighted in red are residues that differ in the core region of the peptide. The asterisk denotes the leader peptide cleavage site.

FIG. 10D depicts structures of cyclothiazomycins A, B, and C.

FIG. 11A depicts an exemplary HPLC trace of cyclothiazomycin C. A sample (spatula tip) of purified cyclothiazomycin C was dissolved in 50% MeOH (B)/aq. 10 mM NH4HCO3 (A) (100 μL). An aliquot (20 μL) was analyzed by HPLC (isocratic 72% B for 35 min). Photodiode array (PDA) detection was used to monitor absorbance (abs) from 190-400 nm. A blank injection was also run and subtracted from the cyclothiazomycin C chromatogram; the resulting spectrum with UV monitoring at 254 nm is shown.

FIG. 11B depicts an exemplary UV spectrum of cyclothiazomycin C. Cyclothiazomycin C exhibits UV absorbance consistent with that reported for cyclothiazomycin A and B1/B2. (1,2) A UV spectrum (PDA) from the HPLC trace at 19.5 min is shown (sh, shoulder).

FIG. 12A depicts an exemplary high resolution Fourier transform mass spectrometry (FT-MS) analysis of cyclothiazomycin C, wherein the m/z scan of purified cyclothiazomycin C showed an ion in the 1⁺ charge state with an observed isotopic m/z value with <2 ppm error from the calculated value for cyclothiazomycin C.

FIG. 12B depicts an exemplary high resolution Fourier transform mass spectrometry (FT-MS) analysis of cyclothiazomycin C for a CID spectrum of m/z 1486. The monoisotopic mass values are given for assigned peak predictions. The number ranges given below the mass values refer to a shorthand notation describing predicted fragments of cyclothiazomycin C. A key for the shorthand notation for the structure of cyclothiazomycin C is given in pictorial format using single letter codes for the amino acids, the residue's N to C position, and lines depicting molecular connectivity within the mature structure. The colors used for the shorthand notation depict the modification present at a particular residue. Purple, thiazoline moieties; green, thioether linkage; cyan, thiazole moieties; red, dehydrated amino acids; orange, pyridine moiety; black, unmodified amino acids.

FIG. 13A depicts the assignments of ¹H and ¹³C resonances are given, wherein the labeling scheme below depicts the lettering system utilized in the table (see FIG. 13D).

FIG. 13B depicts peak assignments as shown directly on the structure of cyclothiazomycin C. Sites where a resonance could not be unambiguously assigned or was not detected are noted. Note that the resonances corresponding to two of the thiazole systems could not be precisely assigned.

FIG. 13C depicts a diagram of connectivity established via 2D correlational experiments. Observed correlations are indicated by red arrows (¹H/¹³C HMBC correlations) or thick black bonds (COSY or TOCSY correlations). Significant germinal ¹H/¹H correlations observed by COSY (blue circles) or TOCSY (green squares) are indicated.

FIG. 13D depicts a table of NMR peak assignments. ¹H NMR shifts of analogous positions on cyclothiazomycin B1 in the same solvent system are shown in the table for comparison. Observed 2D correlations are listed. Abbreviations: Dha, dehydroalanine; Dhb, dehydrobutyrene; Pyr, pyridine; Tzn, thiazoline; Tzl, thiazole; U, unknown; s, singlet; d, doublet; t, triplet; q, quartet; m, multiplet; n.d., not detected; cycloB1, cyclothiazomycin B1, ** ambiguous assignments.

FIG. 14 depicts exemplary NMR spectra of cyclothiazomycin C, wherein complete NMR spectra (¹H, COSY, TOCSY, HSQC, HMBC, and ROESY) are shown.

FIG. 15A depicts the cyclothiazomycin C biosynthetic gene cluster (strain WC-3908, NCBI accession KJ651958 apparently lacked the ctmG gene for the carrying out the [4+2] cycloaddition required for pyridine formation (FIG. 10B). However, BLAST searching found a highly similar gene elsewhere on the WC-3908 chromosome (NCBI accession KJ690935). Interestingly, ctmG from WC-3908 is adjacent to ctmF, which appears to have been duplicated from the rest of the cyclothiazomycin C biosynthetic gene cluster (FIG. 10).

FIG. 15B depicts an amino acid alignment of the CtmG proteins from the cyclothiazomycin A (S. hygroscopicus), cyclothiazomycin B (S. mobaraensis), and cyclothiazomycin C (WC-3908) biosynthetic gene clusters. Below the aligned residues, * represents identical residues, while : and . represent highly and moderately similar residues, respectively.

FIG. 15C depicts an exemplary plot sequence similarity (sum of identical and similar residues/length of longest protein) and identity (identical residues/length of longest protein) between other known formal [4+2] cycloaddition proteins. The gene name and resulting thiopeptide product are given. Values in blue indicate sequence similarity, while green represent sequence identity values.

FIG. 16A depicts genes surrounding the conserved portion of the cyclothiazomycin biosynthetic gene clusters were used as query sequences to identify homologs via BLAST searching. Genes 1-10 represent the genes upstream of the conserved cluster with 1 being the farthest from ctml. Ctml-H are the conserved genes in the clusters (FIG. 4B, NCBI accession number KJ651958) and are highlighted in gray. Genes 11-20 lie downstream of the conserved region.

FIG. 16B depicts BLAST results using the conserved genes from the cyclothiazomycin C gene cluster as query sequences. The best match returned by BLAST and the percent identities are given.

FIG. 17A depicts an exemplary HPLC trace of cyclothiazomycin B. A sample (spatula tip) of purified cyclothiazomycin B was dissolved in 50% MeOH (B)/aq. 10 mM NH4HCO3 (A) (200 μL). An aliquot (27 μL) was analyzed by HPLC (isocratic 75% B for 35 min). Photodiode array (PDA) detection was used to monitor absorbance (abs) from 200-400 nm. A blank injection was also run and subtracted from the cyclothiazomycin B chromatogram; the resulting spectrum with UV monitoring at 254 nm is shown.

FIG. 17B depicts an exemplary UV spectrum of cyclothiazomycin B, wherein the protein exhibits UV absorbance consistent with that previously reported (1) and cyclothiazomycin C (see FIG. 11). A UV spectrum (PDA) from the HPLC trace at 18.6 min is shown (sh, shoulder).

FIG. 18A depicts an exemplary high resolution Fourier transform mass spectrometry (FT-MS) of cyclothiazomycin B, wherein the m/z scan of purified cyclothiazomycin B showed an ion in the 1⁺ charge state with an observed isotopic m/z value with <1 ppm error from the calculated value for cyclothiazomycin B.

FIG. 18B depicts an exemplary CID spectrum of m/z 1528. The monoisotopic mass values are given for assigned peak predictions. The number ranges given below the mass values are a shorthand notation describing predicted fragments of cyclothiazomycin B. A key for the shorthand notation for the structure of cyclothiazomycin B is given in pictorial format using single letter codes for the amino acids, the residue's N to C position, and lines depicting molecular connectivity within the mature structure. The colors used for the shorthand notation depict the modification present at a particular residue. Purple, thiazoline moieties; green, thioether linkage; cyan, thiazole moieties; red, dehydrated amino acids; orange, pyridine moiety; black, unmodified amino acids.

FIG. 19 depicts exemplary MALDI-MS of cell-surface extractions (no media components in spectra) of a single actinomycete cultured using four distinct media. Many metabolites are unique to a given condition.

FIG. 20 depicts a solid-format method wherein eight (1-8) unique actinomycetes are grown under three (a-c) growth conditions on 2×12-well plates. Note that the same strain appears visibly different when cultivated on variable media, as visible evidence of the MS differences in FIG. 19.

FIG. 21A depicts exemplary comparative MALDI-TOF mass spectra showing a solution of anisaldehyde unlabeled (upper spectrum (i)) or labeled with dibrominated probe A2 (lower spectrum (ii)) under the general conditions described above. The unlabeled parent peak is not depicted due to being below the mass threshold of the MALDI-TOF detector.

FIG. 21B depicts exemplary comparative MALDI-TOF mass spectra showing a solution of streptomycin unlabeled (upper spectrum (i))) or labeled with dibrominated probe A2 (lower spectrum (ii))) under the general conditions described above.

FIG. 21C depicts inset of labeling reaction from FIG. 21B showing the characteristic isotope distribution of compounds labeled with a dibrominated probe. This isotope distribution is also evident in FIG. 21A.

FIG. 22 depicts an exemplary MALDI-TOF mass spectrum depicting labeling of kanamycin.

FIG. 23 depicts an exemplary MALDI-TOF mass spectrum depicting labeling of doxorubicin.

FIG. 24 depicts an exemplary MALDI-TOF mass spectrum depicting labeling of vancomycin.

FIG. 25 depicts exemplary comparative MALDI-TOF mass spectra of extracts of S. nodosus, the producer of amphotericin. The top spectrum ((i)) has been labeled with anisaldehyde (probe B1) whereas the lower spectrum ((ii)) is of the unlabeled cell extract.

FIG. 26 depicts a schematic depiction of capture of thiol-bearing compounds using a disulfide resin followed by elution with a thiol (DTT (C1)).

FIG. 27 depicts exemplary comparative MALDI-TOF mass spectra showing crude labeling reaction mixture of thiostrepton and DTT (C1) (spectrum (i)) and the material eluted from the resin with DTT (C1) (spectrum (ii)).

FIG. 28A depicts MALDI-TOF mass spectrum of FK506 labeled with representative thiol probes under thiol-ene coupling conditions.

FIG. 28B depicts MALDI-TOF mass spectrum of quinine labeled with representative thiol probes under thiol-ene coupling conditions.

FIG. 29 depicts biotin-functionalized probes enhancement detection of low-abundance metabolites by affinity enrichment, wherein exemplary MALDI-TOF mass spectra are shown for the crude mixture (subpanel (a)); a sample of FK506 ([M+Na]⁺ m/z=827) as a minor component in a complex extract (subpanel (b)) was subjected to labeling with a biotin-linked thiol probe BT (subpanel (c)); wash fractions of the reaction mixture applied to a streptavidin-linked agarose resin; and elution fraction of the reaction mixture applied to a streptavidin-linked agarose resin (subpanel (e)). For subpanels (d) and (e), the reaction mixture (subpanel (c)) was subsequently evaporated, redissolved in water, and subjected to affinity purification with a streptavidin-linked agarose resin The peak corresponding to labeled material ([M+BT+Na]⁺, m/z=1130) is significantly enhanced upon elution with MeCN/H₂O.

FIG. 30A depicts the structure of thiostrepton with the hypothesized labeling site indicated (blue).

FIG. 30B depicts an exemplary MALDI-TOF mass spectra showing a solution of thiostrepton (front) and the same labeled with tetrazine probe D6 (back).

FIG. 31A depicts the structure of FK506 with the hypothesized labeling site indicated (blue).

FIG. 31B depicts exemplary MALDI-TOF mass spectra showing a solution of FK506 (front) and the same labeled with tetrazine probe D6 (back).

FIG. 32A depicts the structure of rifampicin with the hypothesized labeling site indicated (blue).

FIG. 32B depicts exemplary MALDI-TOF mass spectra showing a solution of rifampicin (front) and the same labeled with tetrazine probe D6 (back).

FIG. 33A depicts the structure of amphotericin B with several possible hypothesized labeling sites indicated (blue).

FIG. 33B depicts exemplary MALDI-TOF mass spectra showing a solution of amphotericin B (spectrum (i)) and the same labeled with tetrazine probe D6 (spectrum (ii)).

FIG. 34 depicts labeling of thiostrepton in the context of an extract of its producing organism, Streptomyces azureus, as shown by exemplary MALDI-TOF mass spectra. The front spectrum shows a CHCl₃ extract of the organism, and the back spectrum shows this extract labeled by probe D6.

FIG. 35 depicts labeling of amphotericins A and B in the context of an extract of their producing organism, Streptomyces nodosus, as shown by exemplary MALDI-TOF mass spectra. The front spectrum shows a MeOH extract of the organism, and the back spectrum shows this extract labeled by probe D6.

FIG. 36 depicts labeling of FK506 in the context of an extract of its producing organism, Streptomyces tsukubaensis, as shown by exemplary MALDI-TOF mass spectra. The front spectrum shows an EtOAc extract of the organism, and the back spectrum shows this extract labeled by probe D6 in MeOH.

FIG. 37 depicts exemplary labeling of unknown peaks in the context of an extract of Streptomyces capuensis NRRL B-12337, as shown by exemplary MALDI-TOF mass spectra of the labeled material.

FIG. 38 depicts exemplary labeling of unknown peaks in the context of an extract of Streptomyces rimosus NRRL WC-3558, as shown by exemplary MALDI-TOF mass spectra of the labeled material.

### DETAILED DESCRIPTION OF THE INVENTION

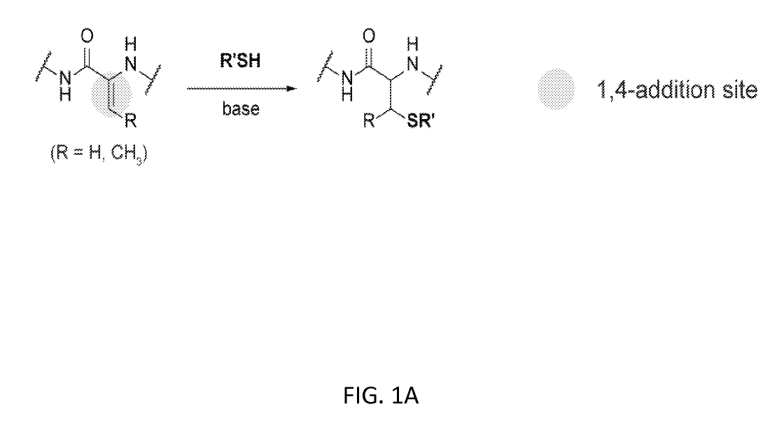

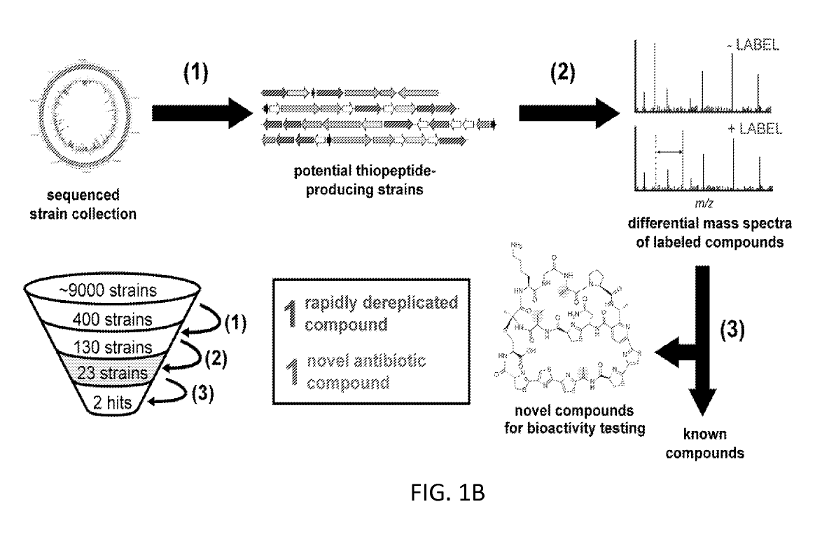

A novel reactivity-based screening method is disclosed herein for natural product discovery that utilizes the intrinsic chemical reactivity of functional groups that are enriched in a target class of metabolites. The reactivity-based screening method enables one to identify, isolate, dereplicate and characterize novel natural products using a combination of bioinformatics and simple chemical probes for modifying reactive functional groups (see, for example, FIG. 1). The method employs specific unique structures found in natural products as useful chemical handles for the their discovery in a variety of organisms (see, for example, FIG. 1A). For organisms in which biosynthetic genes for a specific natural product are clustered together in the genome, the disclosed bioinformatics method enables prioritization of organisms likely to produce the specific natural product, thereby streamlining selection of candidate organisms for the reactivity-based screening method (see, for example, FIG. 1B). The method can find any type of natural product that bears the organic functional group undergoing derivatization.

### Definitions

To aid in understanding the invention, several terms are defined below.

Unless defined otherwise, all technical and scientific terms used herein have the same meaning as commonly understood by one of skill in the art. Although any methods and materials similar to or equivalent to those described herein can be used in the practice or testing of the claims, the exemplary methods and materials are described herein.

Moreover, reference to an element by the indefinite article “a” or “an” does not exclude the possibility that more than one element is present, unless the context clearly requires that there be one and only one element. The indefinite article “a” or “an” thus usually means “at least one.”

The term “about” means within a statistically meaningful range of a value or values such as a stated concentration, length, molecular weight, pH, time frame, temperature, pressure or volume. Such a value or range can be within an order of magnitude, typically within 20%, more typically within 10%, and even more typically within 5% of a given value or range. The allowable variation encompassed by “about” will depend upon the particular system under study.

The terms “comprising,” “having,” “including,” and “containing” are to be construed as open-ended terms (i.e., meaning “including, but not limited to”) unless otherwise noted.

Recitation of ranges of values herein are merely intended to serve as a shorthand method of referring individually to each separate value falling within the range, and includes the endpoint boundaries defining the range, unless otherwise indicated herein, and each separate value is incorporated into the specification as if it were individually recited herein.

The chemical structures described herein are named according to IUPAC nomenclature rules and include art-accepted common names and abbreviations where appropriate. The IUPAC nomenclature can be derived with chemical structure drawing software programs, such as ChemDraw® (PerkinElmer, Inc.), ChemDoodle® (iChemLabs, LLC) and Marvin (ChemAxon Ltd.). The chemical structure controls in the disclosure to the extent that an IUPAC name is misnamed or otherwise conflicts with the chemical structure disclosed herein.

### Rationale and Overview of the Natural Product Discovery Method

A preferred chemical reaction aspect of the reactivity-based screening method for natural product discovery is presented in Scheme I:

NP—[X]ₙ+Y→NP—[X]ₙ₋ₘ[Z]ₘ Scheme I,

wherein NP—[X]ₙ represents a natural product NP having a chemical moiety X that is susceptible to chemical modification by reactivity probe Y to form at least one product adduct NP—[X]ₙ₋ₘ[Z]ₘ in which chemical moiety X reacts with reactivity probe Y to form adduct Z, wherein n ranges from 1 to about 10 and m is at least 1 and m≦n.

In some aspects, a natural product can have a greater number of chemical moieties X than described above. The reactivity-based screening method for natural product discovery based upon Scheme I is highly robust and completely scalable to any natural product comprising NP—[X]ₙ regardless of the value of n. In most cases, however, one can apply Scheme I by focusing on one or two chemical moieties X to enable positive confirmation of candidate natural products for further analysis. As further explained below, the preferred chemical reaction aspect of Scheme I is only one component of the natural product discovery method disclosed herein. Additional components of the disclosed method are required, such as determining the complete structure of the natural product comprising NP—[X]ₙ by physicochemical analysis.

Reactivity probe Y has the structure of Formula (I):

R-L-Q (I),

wherein R is a reactive moiety that reacts with chemical moiety X, L is a linker and Q is a label. In majority of aspects, the stoichiometry of reactive moiety R, linker L and label Q in reactivity probe Y will be 1:1:1 (R:L:Q). In some aspects, the stoichiometry of reactive moiety R, linker L and label Q in reactivity probe Y may differ from 1:1:1 (R:L:Q). For certain aspects of ether formation reactions using silicone-based reagents (for example, SiX₂(L-Q)₂, wherein X is a suitable leaving group (for example, —OH or halogen)) for reactivity probe Y can include a stoichiometry of reactive moiety R, linker L and label Q in reactivity probe Y being 1:2:2 (R:L:Q).

Linker L typically includes at least one covalent bond that links reactive moiety R to label Q. Linker L can include a non-cleavable moiety or a cleavable moiety. Examples of non-cleavable moieties include substituted or nonsubstituted alkyl groups. Examples of cleavable moieties include those cleavable by temperature, light or subsequent chemical reaction, such as pH adjustment, nucleophilic substitution, among others. A preferred linker L includes a non-cleavable alkyl group.

In some aspects, reactivity probe Y has the structure of Formula (I), wherein linker L has zero bond order (that is, L is omitted). In such aspects, label Q is covalently attached directly to an atom present in chemical moiety X to form adduct Z of the at least one product adduct NP—[X]ₙ₋ₘ[Z]ₘ.

Label Q can include any moiety that enables selection, detection, and/or quantitation of the at least one product adduct NP—[X]ₙ₋ₘ[Z]ₘ. In a first aspect, a natural product may be present in low abundance in a sample. In such aspects, Y can preferably include a label Q having an affinity group so one can select and subsequently enrich the at least one product adduct NP—[X]ₙ₋ₘ[Z]ₘ. Exemplary affinity groups include biotin, streptavidin, polyhistine (for example, (His₆)), an unreacted thiol group of dithiothreitol, glutathione-S-transferase (GST), HaloTag®, AviTag, Calmodulin-tag, polyglutamate tag, FLAG-tag, HA-tag, Myc-tag, S-tag, SBP-tag, Softag 3, V5 tag, Xpress tag, a hapten, among others. A preferred label Q for this purpose includes biotin, such as that presented in formula (A):

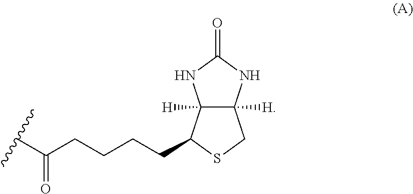

In a second aspect, it is desirable to monitor and quantify the at least one product adduct NP—[X]ₙ₋ₘ[Z]ₘ during purification and subsequent isolation. In such aspects, Y can preferably include a label Q having a detectable group, such as a radiolabel, a fluorescent label, a chemiluminescent label, among others. A preferred label Q for this purpose includes a fluorescent species, such as that presented in formula (B):

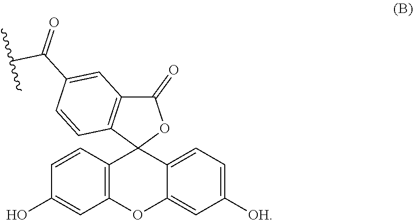 [SMILES: *C(=O)c1ccc2c(c1)C(=O)OC21c2ccc(O)cc2Oc2cc(O)ccc21]

In a third aspect, it is desirable to aid in selecting the at least one product adduct NP—[X]ₙ₋ₘ[Z]ₘ to determine the structure of the natural product comprising NP—[X]ₙ. In such aspects, Y can preferably include a label Q comprising a physicochemical label suitable to select the natural product comprising NP—[X]ₙ for further analysis based upon physical-chemical properties of the at least one product adduct NP—[X]ₙ₋ₘ[Z]ₘ by, for example, NMR or MS. Examples of a physicochemical label include an isotopic label or a mass label. A preferred label Q for this purpose includes a cation mass label amenable to use with MS, such as that presented in Formula (C):

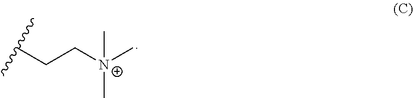 [SMILES: *CC[N+](C)(C)C]

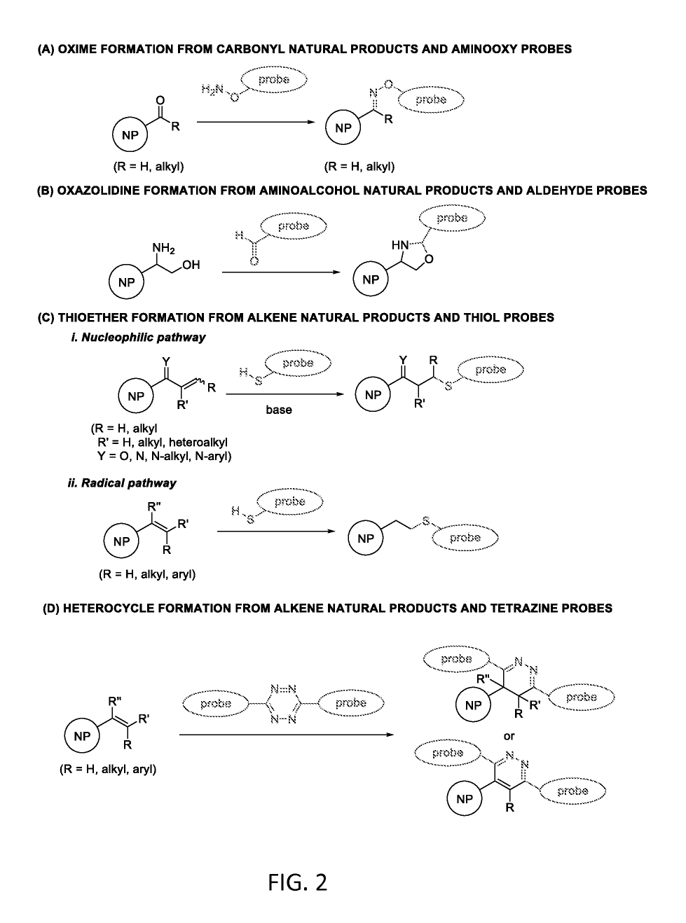

A summary of a set of preferred reactions and their applications with respect to Scheme I is presented in FIG. 2.

One preferred reactivity probe Y having Formula (I) includes an aminooxy compound as reactive moiety R (see Table 1) that can react with carbonyl as chemical moiety X of a natural product NP to form an oxime product (see subpanel (a) of FIG. 2).

TABLE 1
Aminooxy-based reactivity probes Y.

| Reactivity | |
|---|---|
| Probe Y | Structure |
| A1 | 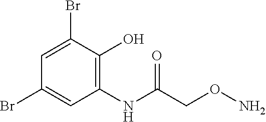 [SMILES: NOCC(=O)Nc1cc(Br)cc(Br)c1O] |
| A2 | 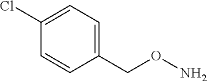 |

One preferred reactivity probe Y having Formula (I) include an aldehyde compound as reactive moiety R (see Table 2) that can react with aminoalcohol or aminothiol as chemical moieties X of a natural product NP to form an oxazolidine or thiazole product (see subpanel (b) of FIG. 2).

TABLE 2
Aldehyde-based reactivity probes Y.

| Reactivity | |
|---|---|
| Probe Y | Structure |
| B1 | 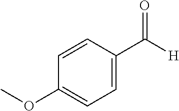 |

Another preferred reactivity probe Y having Formula (I) includes compounds having two reactive groups. The two reactive groups can be identical or different. An example of a compound having two identical reactive groups is dithiothreitol, which includes two reactive thiol groups. For example, following reaction of reactivity probe Y having two thiol groups with chemical moiety X of NP—[X]ₙ, one unreacted thiol group remains available for further reaction. In such cases, the unreacted thiol group can be considered as label Q as described supra. In these cases, the unreacted thiol group can enable selection, detection, quantitation and/or determination of the structure of the at least one product adduct NP—[X]ₙ₋ₘ[Z]ₘ. Thus, the unreacted thiol group can be used as an affinity group so one can select and subsequently enrich the at least one product adduct NP—[X]ₙ₋ₘ[Z]ₘ using, for example, thiol capture resins. Such resins include thiol groups for forming covalent bonds with the unreacted thiol group present in the at least one product adduct NP—[X]ₙ₋ₘ[Z]ₘ. Similarly, the unreacted thiol group present in the at least one product adduct NP—[X]ₙ₋ₘ[Z]ₘ can be used in subsequent reactions with an extrinsic label (Q′) that includes an unreacted thiol group coupled to a detectable group (for example, a radiolabel, a fluorescent label, a chemiluminescent label, among others) or a physicochemical label (for example, an isotopic label or a mass label). In the latter context, dithiothreitol can serve as a physicochemical label (for example, a mass label) owing to its unique mass signature following reaction with X of NP—[X]ₙ.

A preferred set of reactivity probes Y use a thiol as reactive moiety R (Table 3) for reaction with alkene natural products (see subpanel (c) of FIG. 2).

TABLE 3
Thiol-based reactivity probes Y

| Re- | |
|---|---|
| activity | |
| Probe Y | Structure |
| C1 | 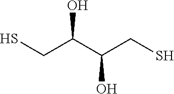 |
| C2 | 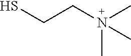 |
| C3 | 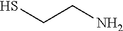 |
| C6 | 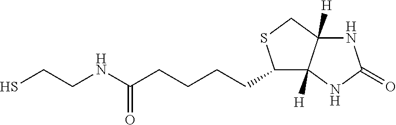 [SMILES: O=C(CCCC[C@@H]1SC[C@@H]2NC(=O)N[C@@H]21)NCCS] |
| C7 | 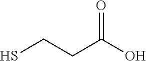 |
| C8 | 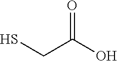 |
| C9 | 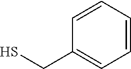 |
| C10 | 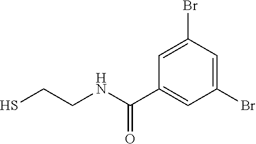 [SMILES: O=C(NCCS)c1cc(Br)cc(Br)c1] |
| C11 | 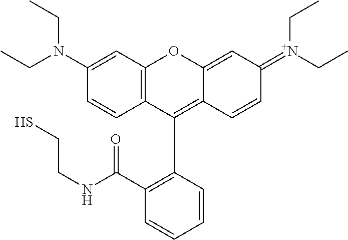 [SMILES: CCN(CC)c1ccc2c(-c3ccccc3C(=O)NCCS)c3ccc(=[N+](CC)CC)cc-3oc2c1] |

By varying reaction conditions, these reagents can target an array of electron-rich or electron-poor alkenes. As discussed infra, the utility of dithiothreitol (C1) as a reactivity probe Y has been described. Probe C2 is a simple thiocholine; the incorporation of permanent cations results in substantial analyte MS signal enhancement. As many NPs exist at exceptionally low levels, this method will facilitate their detection. Probe C3 is a bifunctional probe carrying an amine to enable subsequent reaction with one of a variety of labels Q. Compounds labeled with biotin-bearing C6, can be enriched via affinity chromatography, allowing removal of non-labeled compounds and retention of labeled metabolites. C7 and C8 have the added anticipated benefits of enhancing compound solubility in hydrophilic solvents. C9 has the added anticipated benefits of enhancing compound solubility in hydrophobic solvents. Probe C10 bears a dibrominated moiety that gives rise to characteristic isotope peaks in mass spectra, allowing direct detection of labeling without the need for spectral comparisons. This dibromo probe strategy has been validated in the selective tagging and MS analysis of proteins. Probe C11 is rhodamine-linked (although any suitable fluorophore can be substituted), allowing for a selective UV-HPLC visualization of labeled compounds. Software is known in the art for automated detection of tagged molecules.

One preferred reactivity probe Y having Formula (I) include a tetrazine compound as reactive moiety R (see Table 4) that can react with an alkene as a chemical moiety X of a natural product NP to form a heterocycle product (see subpanel (d) of FIG. 2).

TABLE 4
Aldehyde-based reactivity probes Y.

| Reactivity | |
|---|---|
| Probe Y | Structure |
| D1 | 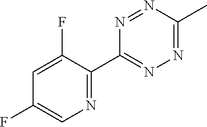 |
| D2 | 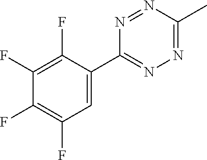 [SMILES: Cc1nnc(-c2cc(F)c(F)c(F)c2F)nn1] |
| D3 | 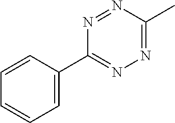 |
| D4 | 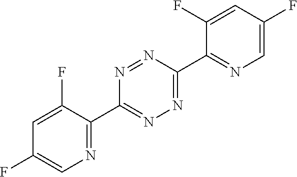 [SMILES: Fc1cnc(-c2nnc(-c3ncc(F)cc3F)nn2)c(F)c1] |
| D5 | 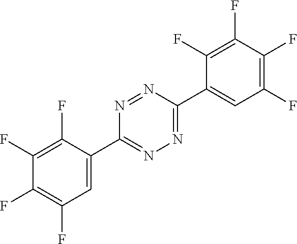 [SMILES: Fc1cc(-c2nnc(-c3cc(F)c(F)c(F)c3F)nn2)c(F)c(F)c1F] |
| D6 | 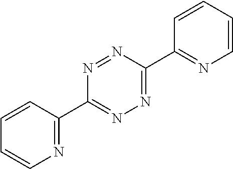 [SMILES: c1ccc(-c2nnc(-c3ccccn3)nn2)nc1] |

A natural product NP—[X]ₙ can include a variety of different chemical moieties X. Accordingly, the reactivity-based screening method contemplates a corresponding variety of reactivity probes Y, wherein at least one reactivity probe Y can react with at least one chemical moiety X to form at least one adduct Z in the final product NP—[X]ₙ₋ₘ[Z]ₘ of Scheme I. Based upon the reactivity probes Y presented in Tables 1-4, a summary listing several exemplary species of chemical moieties X found in natural products and corresponding reactivity moieties R of Y suitable for reacting with chemical moieties X to form Z is presented in Table 5. The variations of Scheme I summarized in Table 5 are robust reactions well known in the art.

TABLE 5
Exemplary X, Y and Z moieties of Scheme I.

| Rxn¹ | X² | Y³ | Z⁴ |
|---|---|---|---|
| 1 | 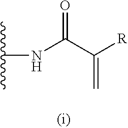 | 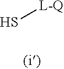 | 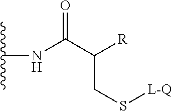 |
| 2 | 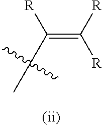 | 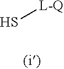 | 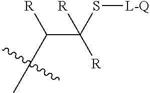 |
| 3 | 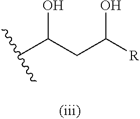 | 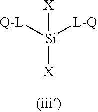 | 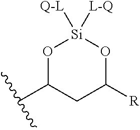 |
| 4 | 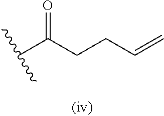 [SMILES: *C(=O)CCC=C] | 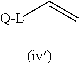 | 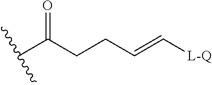 |
| 5 | 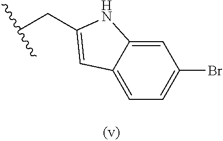 [SMILES: *Cc1cc2ccc(Br)cc2[nH]1] | Q-L-Met (v′) | 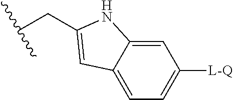 |
| 6 | 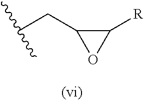 | 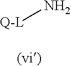 | 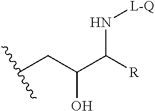 |
| 7 | 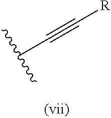 | 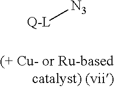 | 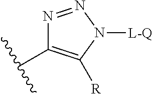 |
| 8 | 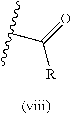 | 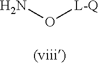 | 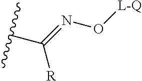 |
| 9 | 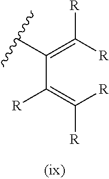 | 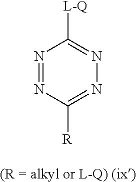 | 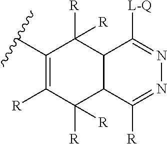 |
| 10 | 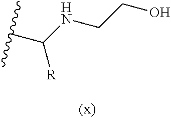 | 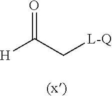 | 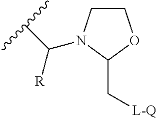 |
| 11 | 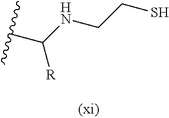 | 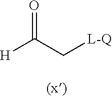 | 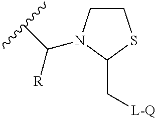 |

¹Reaction (Rxn) summary for each exemplary variation of Scheme I is as follows: (1) 1,4-addition into α,β-unsaturated carbonyl/imine in the presence of thiol conjugation agent (HS-L-Q) and a base; (2) Strieter thio-ene coupling reaction carried out in presence of a thiol conjugation agent (HS-L-Q); (3) ether formation in the presence of SiX₂(L-Q)₂; (4) cross-metathesis reaction in the presence of an alkenyl-containing R-L-Q; (5) cross-coupling reaction; (6) epoxide ring opening reaction; (7) cycloaddition reaction on an alkyne in the
presence of an azide reactive moiety in DMF (Cu-based catalyst (Cu(I), or Cu(II) with reductant) for terminal alkynes and Ru-based catalyst for internal alkynes, RT, 24 h); (8) oxime formation from an aldehyde or ketone using an aminooxy derivative and (9) Diels-Alder reaction with a dienophile (for example, a tetrazine derivative); (10) reaction of 1,2-aminoalcohol (or 1,3-aminoalcohol) by aldehyde-mediated oxazolidine formation; and (11) reaction of 1,2-aminothiol (or 1,3-aminothiol) by aldehyde-mediated thiazole formation.
²X moiety of NP-[X]ₙ is illustrated, where the wavy line ( [symbol] ) depicts at least one scaffold connection to the remainder of the natural product and the R group(s) depict(s) additional scaffold attachments or terminal groups including connecting atoms selected from H, C, N, O, P and S.
³Y is illustrated in R-L-Q format, wherein the explicit structure of the reactive moiety R is shown.
⁴Z illustrates the predicted structure of the at least one adduct Z in the final product NP-[X]ₙ₋ₘ[Z]ₘ.

In a first aspect, natural product NP—[X]ₙ can include only one chemical moiety X (that is, n=1). In such aspects, a single type of reactivity probe Y is suitable for reacting with NP—[X]ₙ to form NP—Z (that is, NP—[X]ₙ₋ₘ[Z]ₘ, wherein n=1 and m=1). In a second aspect, natural product NP—[X]ₙ can include two or more of the same type of chemical moiety X, such as NP—[X]₂ (that is, where n=2). In such aspects, one or two different types of reactivity probes Y, such as Y¹ or a combination of Y¹ and Y², can be used to in reactions with NP—[X]₂ to form NP—[Z¹]₂ or NP—[Z¹,Z²], respectively. In a third aspect, natural product NP—[X]ₙ can includes two or more of the different types of chemical moiety X, such as NP—[X¹,X²] (that is, where n=2). In such aspects, different types of reactivity probes Y¹ and Y², can be used singly or in combination in reactions with NP—[X¹,X²] to form NP—[Z¹,X²], NP—[X¹,Z²] or NP—[Z¹,Z²], wherein Y¹ displays reactivity to only X¹ and Y² displays reactivity to only X².

Natural products are present in all organisms. Accordingly, the reactivity-based screening method for natural product discovery is applicable to any organism. Exemplary organisms include bacteria, fungi, plant cells, and animal cells as suitable starting materials for the discovery pipeline. To the extent that certain parasites, such as viroid's, sinusoids, and viruses (among others), modify host cells to produce altered natural products, host cells harboring such parasites are also suitable starting materials for the discovery pipeline.

Organisms (or cells) are typically treated in a manner to prepare a sample including complex cellular metabolite mixture. In some aspects, the complex cellular metabolite mixture can include a crude or partially purified total cell extract. In other aspects, the complex cellular metabolite mixture can include cell surface-associated metabolites (for example, exported metabolites). Preferred organic solvents include chloroform and volatile alcohols, such as methanol, various isomeric forms of butanol, and various isomeric forms of propanol, among others. Preferred organic solvents include chloroform, methanol, n-butanol and isopropanol. The choice of organic solvent can depend upon the organism subjected to the non-lytic cell surface-associated exported metabolite as well as the physicochemical properties of the compound(s) undergoing extraction.

The complex cellular metabolite mixture suspected to include at least one natural product NP—[X]ₙ is reacted with at least one reactivity probe Y to form at least at least one product adduct NP—[X]ₙ₋ₘ[Z]ₘ according to Scheme I. Generally, reactivity probe Y is selected such that a natural product NP—[X]ₙ and the at least one product adduct NP—[X]ₙ₋ₘ[Z]ₘ differ in at least one physicochemical characteristic. A preferred physicochemical characteristic difference between natural product NP—[X]ₙ and the at least one product adduct NP—[X]ₙ₋ₘ[Z]ₘ is a mass difference between these two species due to the presence of at least label Q present in adduct Z. Such a mass difference can be readily detected using differential mass spectrometry (MS).

Accordingly, two MS spectra are obtained corresponding to the complex cellular metabolite mixture before and after reaction with reactivity probe Y in Scheme I and a difference MS spectra is generated either visually or computationally. Molecular species of natural products consistent with a mass spectra shift are identified from the difference MS spectra. Because previously discovered natural products are known, one can readily generate a priori a set of predicted mass values of product adducts NP—[X]ₙ₋ₘ[Z]ₘ for known natural products based upon the specific reactivity probe(s) Y used with the complex cellular metabolite mixture in Scheme I. Those mass values corresponding to NP—[X]ₙ₋ₘ[Z]ₘ adducts of previously discovered natural products appearing in the difference MS spectra are removed from consideration (“dereplicated”), leaving the remaining molecular species as candidate novel natural products available for further detailed structural characterization.

The robust power of this reactivity-based screening method therefore lies in one identifying and dereplicating previously known natural products from the complex cellular metabolite mixture before one begins detailed structural characterization of candidate natural products. Since the majority of the energy, time and expense in natural product discovery arise during the detailed structural characterization stage, the reactivity-based screening method disclosed herein assures one that subsequent work on the selected, dereplicated population of candidate natural products will focus on viable, novel products rather than previously discovered products.

In some respects, the dereplication step is optional and can be omitted in some instances of the discovery screening strategy. If one works with a well-characterized, popular strain, dereplication is necessary to expedite the discovery process. However, if one works with unusual or inconvenient strains where there are no previously identified natural product compounds known, dereplication cannot be accomplished as every identified natural product is novel. Those strains may nevertheless have a gene cluster similar to a known compound; thus, one can obtain insights about the structure/function from genomic analysis. If the screened strain has an identical gene cluster to a known compound, there is a very high probability that the strain will make the same compound. In those instances, dereplication step is not only feasible, but preferable to perform as part of the discovery strategy.

The biosynthesis of natural products is brought about by the coordinated action of several enzymes encoded in variety of genes. For certain organisms, such as bacteria and fungi, a great majority of the genes encoding enzymes responsible for biosynthesis of a particular natural product are often clustered together in the genome. Though the linkage relationship for each of the natural product gene clusters can vary, it is common to find two or more genes for a given natural product biosynthetic pathway within linkage proximity to each other, such as, for example, a range of about ten open reading frames of each other. The discovery pipeline begins in these aspects with a bioinformatics survey for strains of a given organism predicted to be capable of producing a particular natural product having a chemical moiety X generated by the concerted action of two or more biosynthetic enzymes.

In one aspect, this bioinformatics-based strain prioritization includes three steps. The first step includes populating a list of strains encoding a first enzyme for the biosynthesis pathway of the chemical moiety X. The second step includes reducing the list of strains encoding a second enzyme for the biosynthesis pathway of the chemical moiety X to yield a refined list of strains, wherein the second enzyme is encoded by a gene having proximity to a gene encoding the first enzyme (for example, the genes encoding the first and second enzymes range about ten open reading frames apart in the chromosome). The third step includes identifying precursor peptide products of the first enzyme from the refined list of strains.

After the bioinformatics-based strain prioritization is performed, a select number of the prioritized strains are cultivated for preparing complex cellular metabolite mixture(s). The complex cellular metabolite mixture(s) suspected to include natural product NP—[X]ₙ are surveyed using the reactivity-based screening method of Scheme I with at least one reactivity probe Y, and preferably with a platform including a plurality of reactivity probes Y.

The combination of bioinformatics-guided predictive methodology to prioritize organism candidates for subsequent analysis dramatically improves the efficiency of reactivity-based screening method for natural product discovery. The bioinformatics-based prioritization method permits one to focus on those candidate organisms likely to produce natural products having a specific chemical moiety X, which is a product of the desired, targeted biosynthetic pathway of interest. One can then focus efforts on using the prioritized collection of organisms using highly specific reactivity probes Y for chemical moiety X according to the reactivity-based screening method of Scheme I in conjunction with differential MS as described above.

### Application of the Methods to Discover a New Natural Product Having Dehydrated Amino Acids

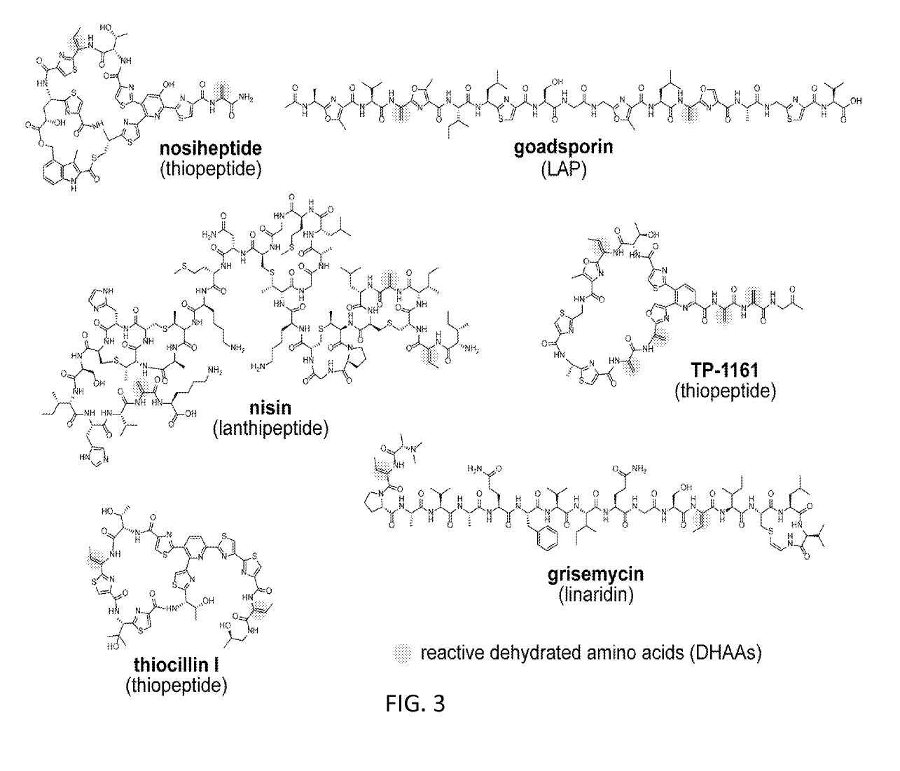

In the proof of principle example, the method employs dehydrated amino acids (DHAAs) as useful chemical handles for the discovery of natural products, as DHAAs are frequently found in natural products, including thiopeptides, lanthipeptides and linaridins, among others (FIG. 3). Thio nucleophiles participate in 1,4-addition into α,β-unsaturated carbonyl/imine DHAAs under mild conditions to yield covalent thioether adducts (FIG. 1A).

A combination of bioinformatics and nucleophilic 1,4-addition chemistry is disclosed for the rapid labeling, discovery, and dereplication of DHAA-containing natural products (FIG. 1B) by reactivity-based screening. The discovery pipeline begins with a bioinformatic survey for strains of Actinobacteria predicted to be capable of producing a DHAA-containing natural product. (FIG. 1B, Step 1, vide infra for specifics on the bioinformatics-based strain prioritization). After cultivation, the exported metabolites from the prioritized Actinobacteria are extracted with organic solvent using a non-lytic procedure (see Examples). A portion of this cell-surface extract then undergoes treatment with dithiothreitol (DTT) in the presence of a Bronsted base. DTT was chosen as the thiol probe owing to its low cost and ubiquity in natural product discovery laboratories. If reactive DHAA moieties are present in the cell-surface extract, the resulting DTT adducts increase the mass of the exported metabolite by multiples of 154.0 Da (FIG. 1B, Step 2). Differential mass spectrometry between the unreacted control and the DTT-reacted extracts readily identifies the compounds containing DHAAs within a pre-determined mass range. The molecular mass, number of DTT additions, and analysis of tandem mass spectra, combined with the initial bioinformatic prediction of DHAA-containing natural products, permits a rapid determination of compound novelty. Known compounds are removed from further analysis at this step, leaving only compounds with a high probability of novelty for further structural and functional characterization, which is considerably more time-consuming (FIG. 1B, Step 3). To determine if the above proposed discovery pipeline was viable, we sought to discover a novel DHAA-containing thiopeptide via bioinformatic prioritization and reactivity-based screening utilizing nucleophilic 1,4-addition chemistry.

### Validation of the DTT-Labeling Strategy

With the ultimate goal of using the above-described DTT-labeling method to discover new natural products, we first sought to establish whether the DTT-labeling method was a viable and operationally simple route to rapidly screen organic extracts for compounds of interest. We utilized two DHAA-containing natural products, thiostrepton and geobacillin I, for method development and validation.

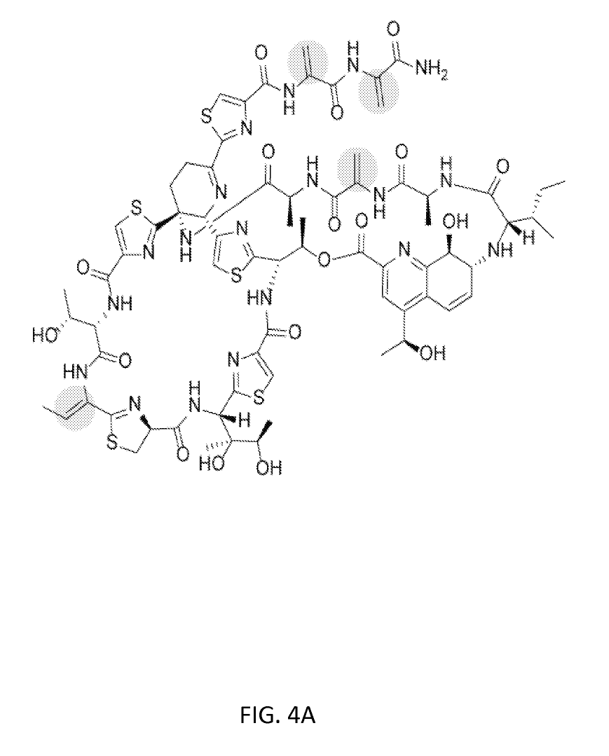

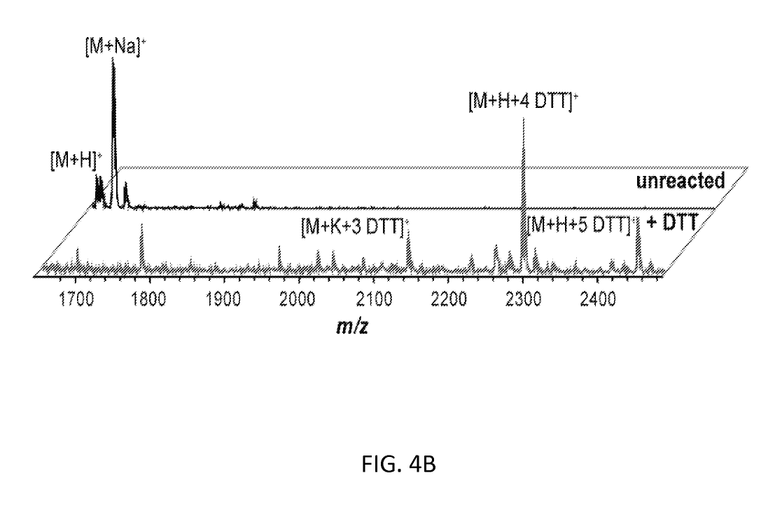

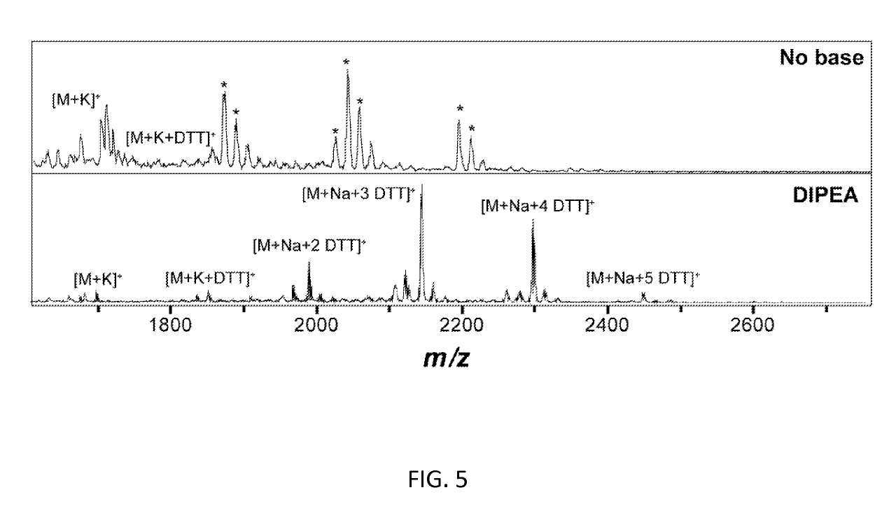

Thiostrepton is a thiopeptide produced by Streptomyces azureus ATCC 14921 (among others). Notably, the highly-modified scaffold of thiostrepton contains four DHAAs where labeling can occur: three dehydroalanine residues and one dehydrobutyrine (FIG. 4A). To test the method, reactions were conducted using commercially-obtained thiostrepton, DTT, and either diisopropylethylamine (DIPEA) or no base at 23° C. for 16 h in a 1:1 mixture of chloroform and methanol. The authentic thiostrepton standard and the DTT-reacted samples were then subjected to matrix-assisted laser desorption/ionization time-of-flight mass spectrometry (MALDI-TOF MS) analysis. The peaks corresponding to unmodified thiostrepton (m/z 1664.4 Da) were supplanted in the DTT-reacted sample by peaks corresponding to the addition of multiple DTT labels, suggesting the successful addition of DTT into the reactive alkenes (FIG. 5). The addition of base enhanced the DTT-labeling reaction. Other bases, including triethylamine and 1,8-diazabicycloundec-7-ene (DBU), were tested and labeling occurred similarly to the reactions using DIPEA. A range of DIPEA concentrations were tested (10-50 mM) and the extent of labeling did not greatly vary. Therefore all further experiments employed 10 mM DIPEA.

To confirm DTT-labeling of thiostrepton could be observed by MALDI-TOF MS in the context of a more complex biological mixture, we subjected an organic cell-surface extract of S. azureus ATCC 14921 (thiostrepton producer) to the above labeling reaction. Analogous to the pure thiostrepton sample, comparison of the crude extract with the DTT-labeled extraction again showed the appearance of multiple DTT adducts, with the tetra-adduct being the primary species; a higher extent of labeling was seen here due to the larger relative excess of the labeling reagents in the context of a biological extract (FIG. 4C). Although thiostrepton contains only 4 reactive DHAA sites, a minor 5^{th} adduct was observed in both the commercially available and extracted samples, presumably from reaction with another electrophilic site. Thiostrepton possesses an additional alkene that is conjugated to pyridine within the quinaldic acid moiety; we hypothesize that addition of DTT may have occurred at this site, given the literature precedent for addition of thiols to aromatic-conjugated alkenes. Importantly, the appearance of this low-intensity ion does not complicate detection or interpretation of the labeled analyte.

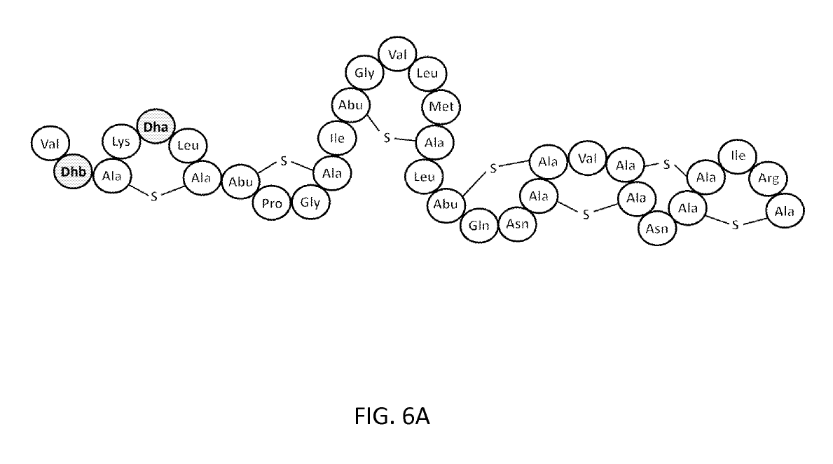

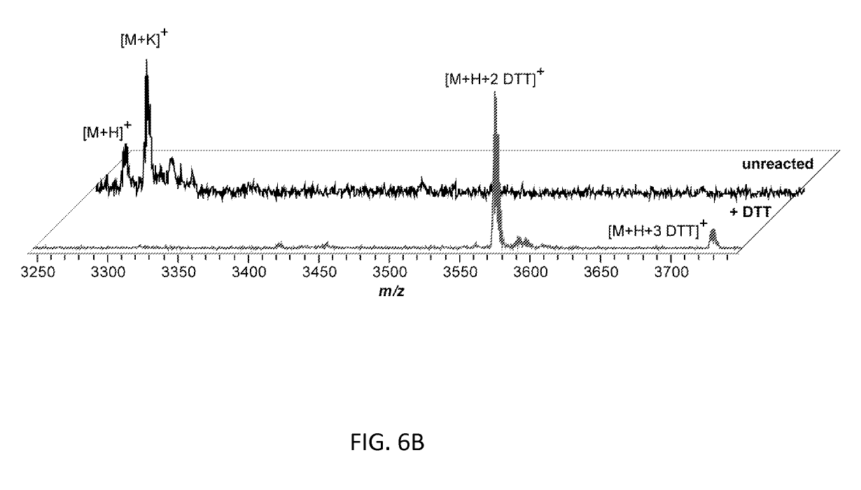

Lanthipeptides are ribosomally synthesized and post-translationally modified peptide natural products (RiPPs) that are easily identified using bioinformatics and frequently contain DHAAs. To test if the reactivity-based screening method could also be used to identify other classes of natural products in varied bacterial extracts, we attempted to label the lanthipeptide geobacillin I. Geobacillin I, a nisin analogue, is produced by Geobacillus sp. M10EXG (FIG. 6A). Upon subjecting an organic cell-surface extract from Geobacillus sp. M10EXG to our labeling conditions, a mass corresponding to 2 DTT adducts was prominently observed; a third adduct was visible but of very low intensity (FIG. 6B). Only two reactive DHAA sites are present in geobacillin I: a dehydroalanine and a dehydrobutyrine. However, transient DHAA sites occur in the biosynthesis of the lanthionine rings, which are formed by intramolecular 1,4-addition of cysteines to DHAAs. We hypothesize, accordingly, that a small percentage of the geobacillin present in the extract may have an unformed lanthionine ring, leaving a free reactive site available for DTT-labeling. Again, even under stoichiometrically forcing conditions, this extract adduct was of only minor abundance and thus did not interfere with compound detection or analysis.

### Bioinformatics Guided Strain Prioritization

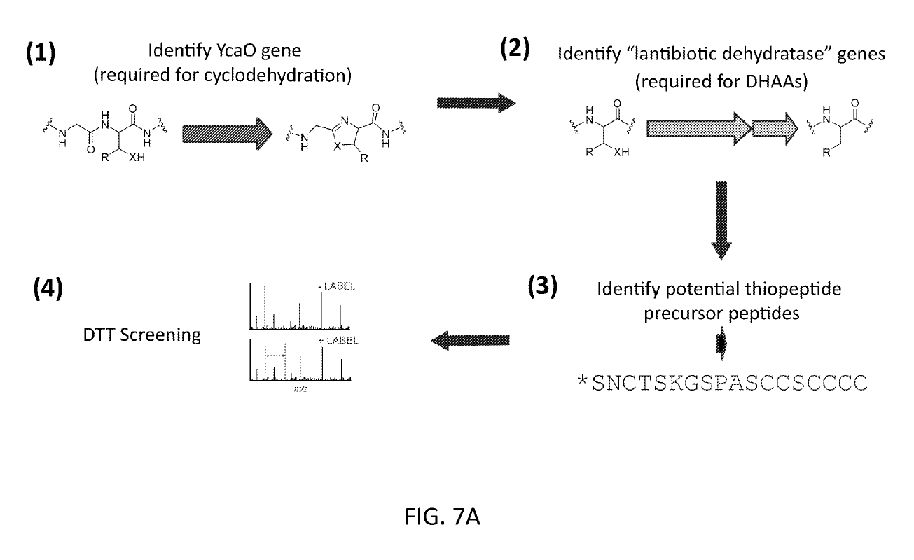

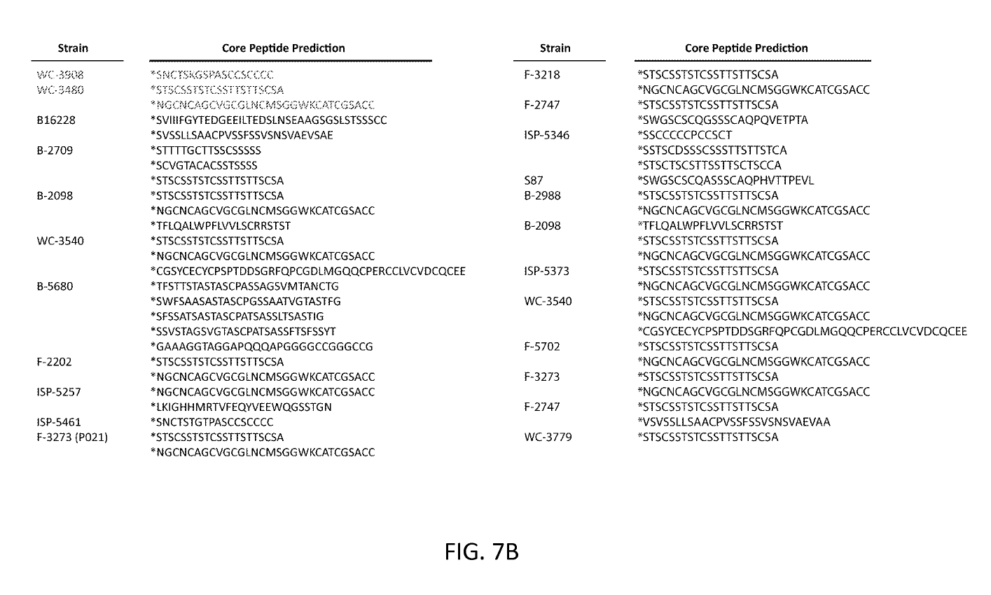

Like lanthipeptides, thiopeptides are RiPPs and the biosynthetic genes responsible for their production are often clustered, rendering them identifiable by sequence similarity searching. From the perspective of the present study, we sought to prioritize bacterial strains for subsequent screening based on the presence of biosynthetic genes capable of installing DHAAs (often misleadingly annotated as “lantibiotic dehydratases”). These genes, however, can be found in a variety of other natural product gene clusters and not exclusively in thiopeptide clusters. Therefore, we first identified clusters that encode for the YcaO cyclodehydratase protein that is necessary for the biosynthesis of all thiazole/oxazole-modified microcin natural products, of which thiopeptides can be broadly categorized. Strains containing a YcaO cyclodehydratase were analyzed further for the local co-occurrence of genes encoding a “lantibiotic dehydratase” (for the production of DHAAs) and a thiopeptide-like precursor peptide (FIG. 7A). 130 unique strains of recently sequenced (in-house) Actinobacteria from the Northern Regional Research Laboratory collection (NRRL), which is curated by the Agricultural Research Service under the supervision of the U.S. Department of Agriculture (USDA/ARS), were predicted to have the genetic capacity to produce a DHAA-containing thiopeptide (FIG. 1B). The precursor peptide sequences from these clusters were then used to estimate the masses of the final natural products for dereplication and characterization purposes (FIG. 7B). These strains were then subjected to reactivity-based screening with DTT and DIPEA to discover a novel thiopeptide.

### MS-Based Screening of Prioritized Strains

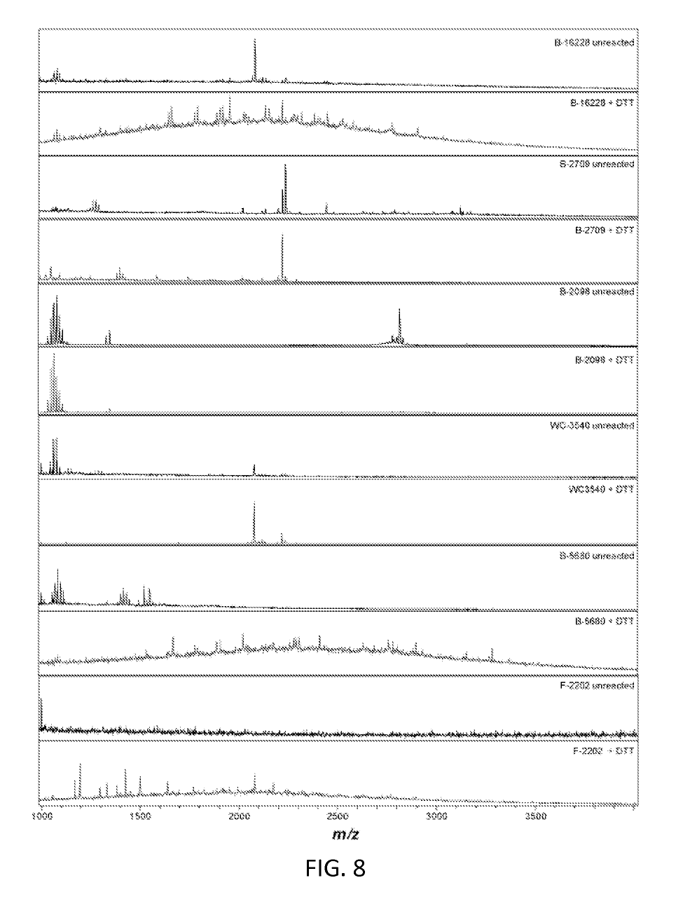

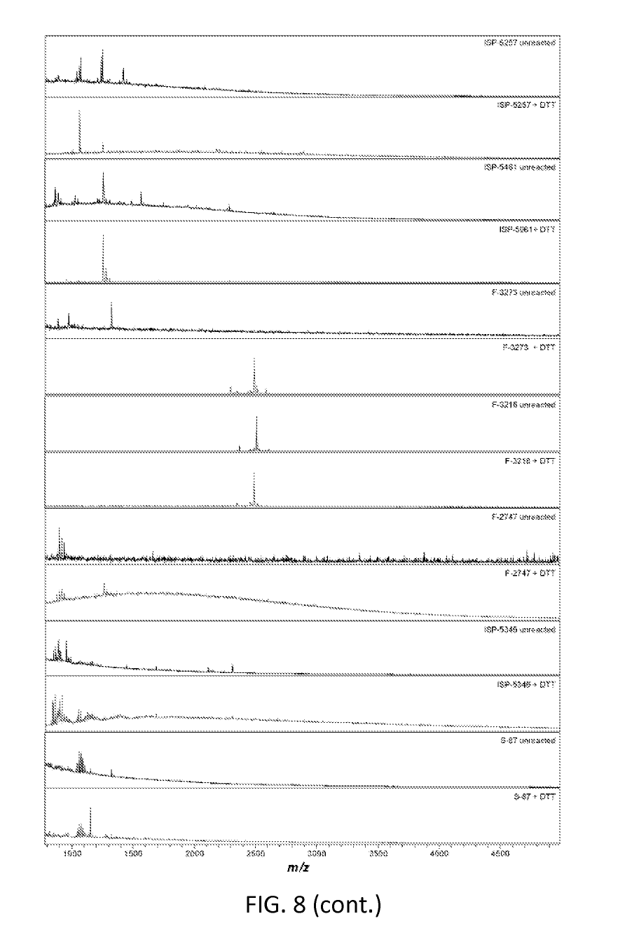

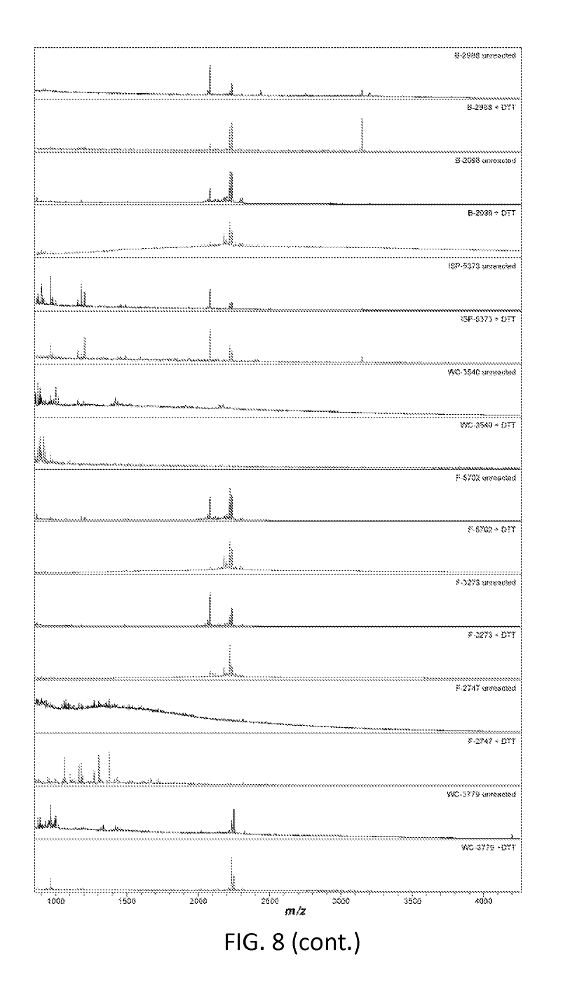

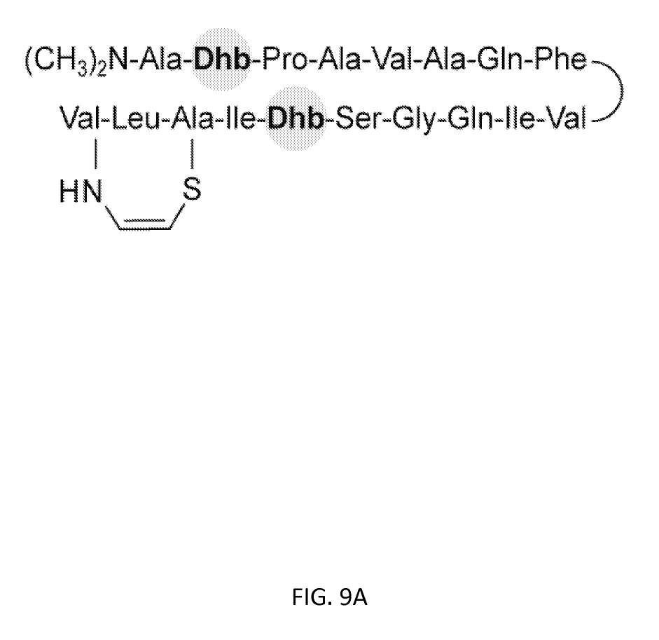

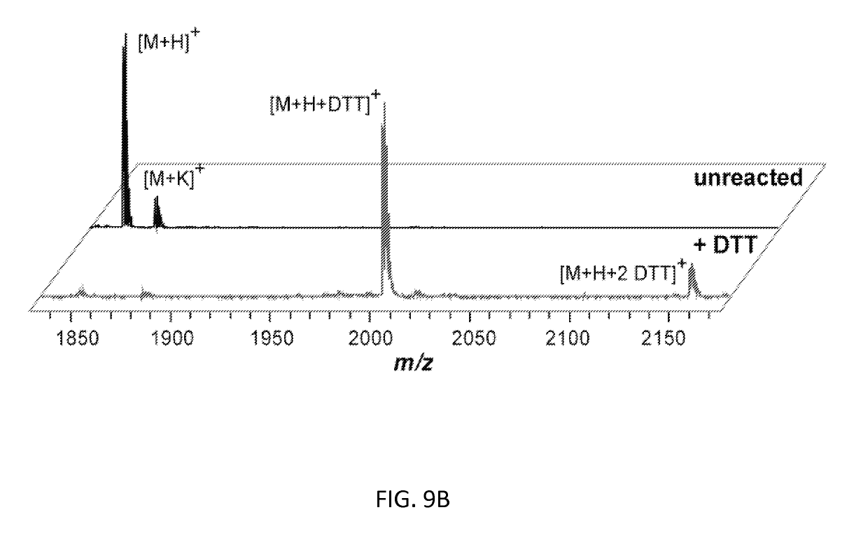

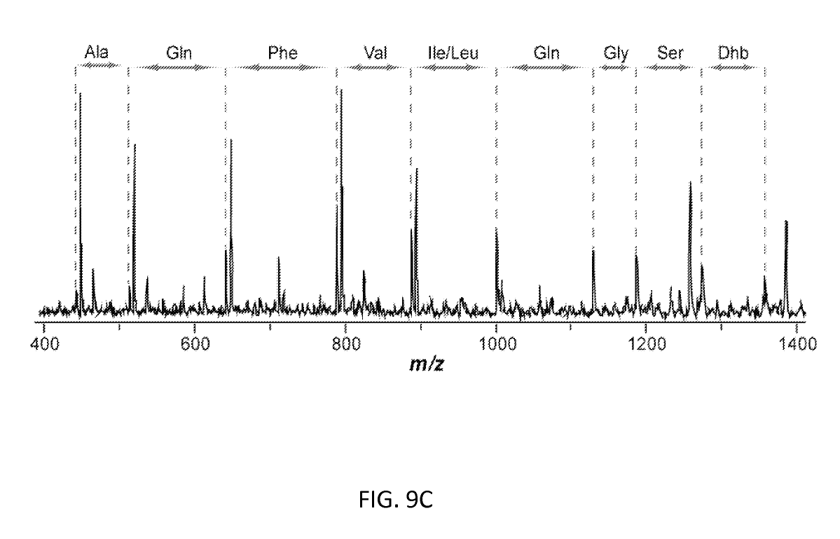

Twenty-three of the prioritized strains with novel precursor peptide sequences were selected for screening by DTT-labeling (FIG. 8). We first noticed a sample containing 1-2 DTT adducts on an exported metabolite with a mass of [M+H]⁺, m/z 1855.0 Da. While we were intentionally blind to which of the Actinobacteria strains were undergoing analysis, after labeling we established that this particular extract originated from Streptomyces griseus subsp. griseus, and the labeled mass did not correlate with the expected mass of the predicted thiopeptide from this strain. However, Streptomyces griseus subsp. griseus is a known producer of grisemycin (FIG. 9A), of which the mass of the labeled natural product did correlate (FIG. 9). MS/MS fragmentation analysis yielded a seven amino acid sequence tag confirming the identity of the compound as grisemycin (FIG. 9B). The labeling and identification of grisemycin, a member of the linaridin class of natural products, further validated our reactivity-based screen while also highlighting the usefulness of bioinformatic integration to rapidly dereplicate known compounds.

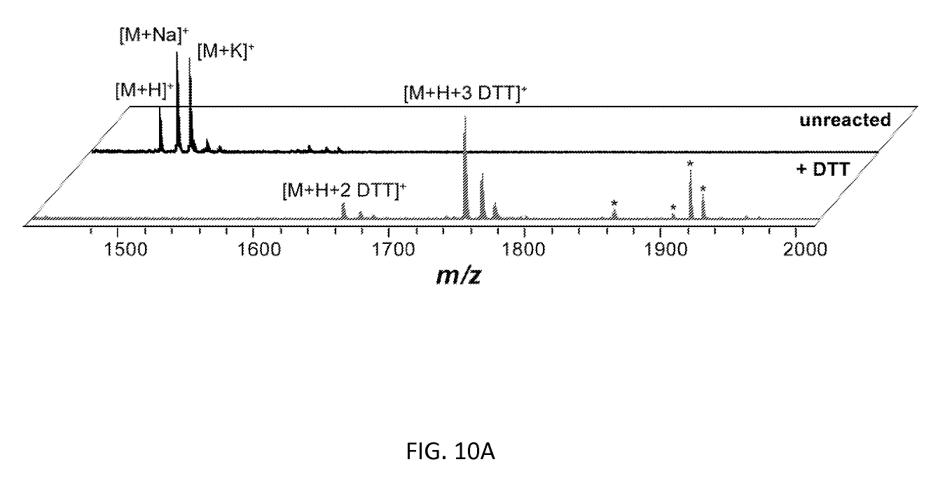

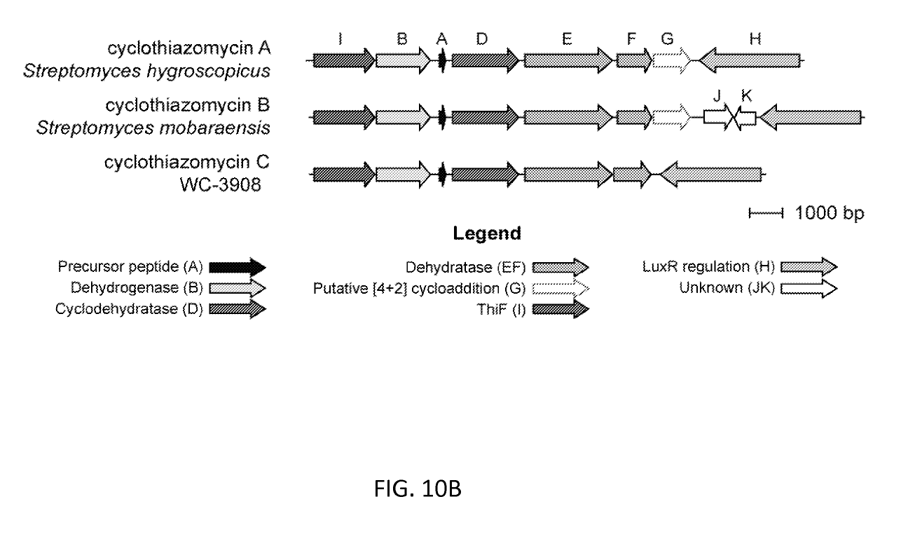

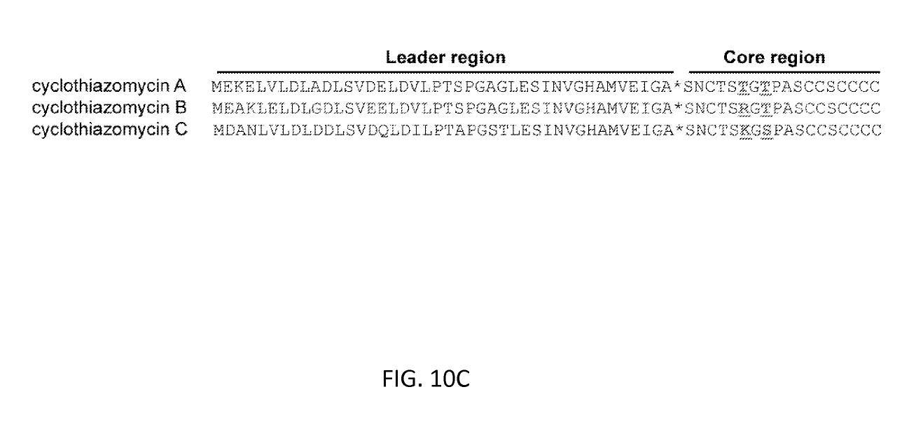

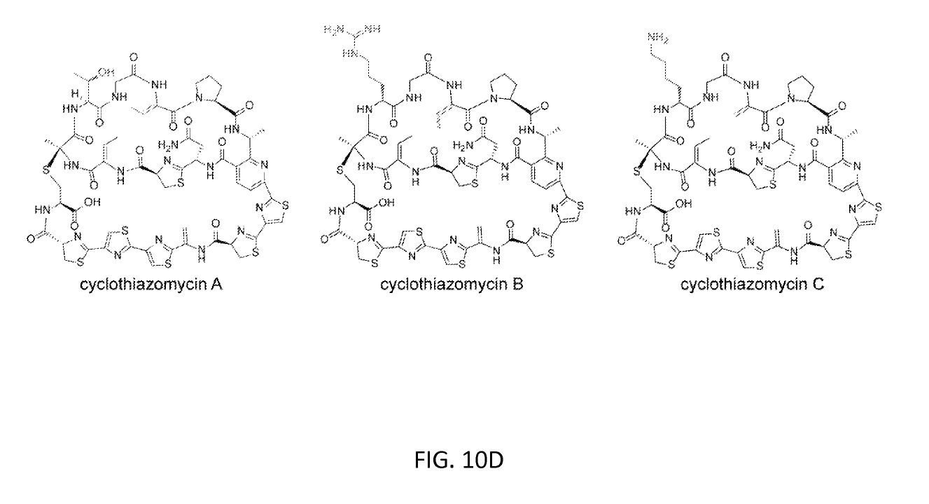

The organic cell-surface extract from a separate sample contained a compound ([M+H]⁺, m/z 1486.3 Da) that underwent labeling to contain primarily three DTT adducts (FIG. 10A). This mass correlated well with the predicted mass of a hypothetical thiopeptide from NRRL strain WC-3908. The thiopeptide gene cluster from WC-3908 was similar to the gene clusters responsible for the production of the thiopeptides cyclothiazomycin A, originally termed 5102-I and cyclothiazomycin B (FIG. 10B). The core region of the precursor peptide (i.e. the portion that undergoes enzymatic tailoring to yield the mature natural product) from WC-3908 differed by two amino acids from the precursor peptides of cyclothiazomycin A and B (FIG. 10C). Accordingly, we designated the WC-3908 thiopeptide cyclothiazomycin C. Given the known structures of cyclothiazomycin A and B, we could accurately predict the structure of cyclothiazomycin C, which was in agreement with the labeling results (FIG. 10D).

### Verification of the Cyclothiazomycin C Structure

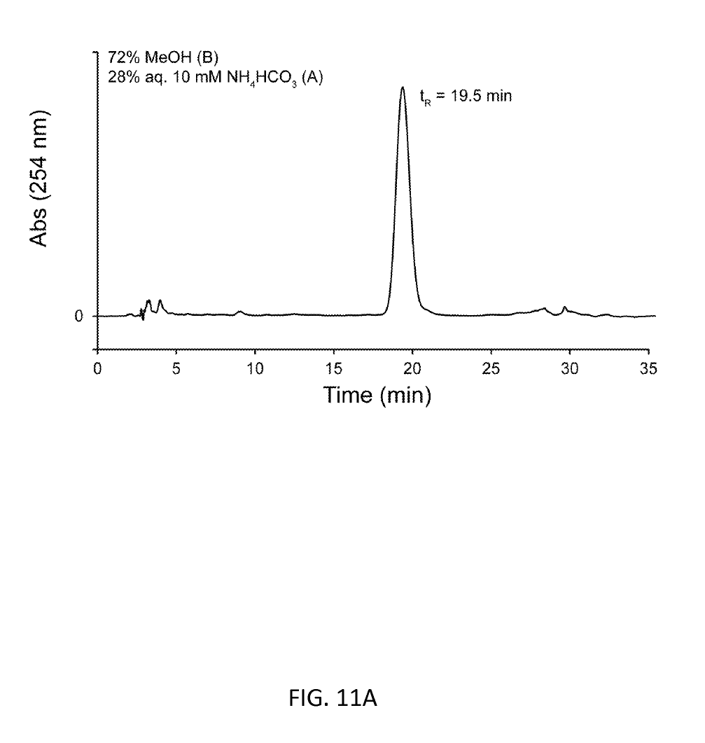

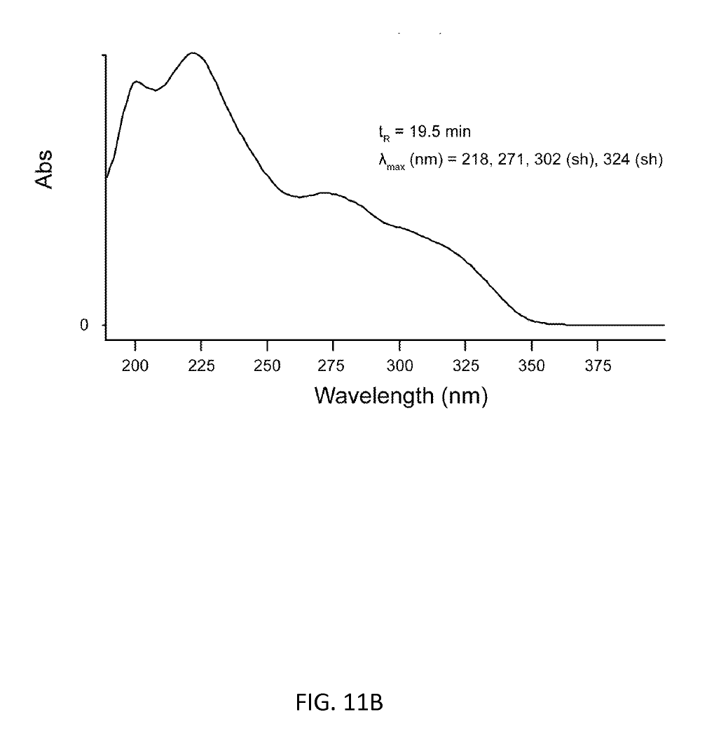

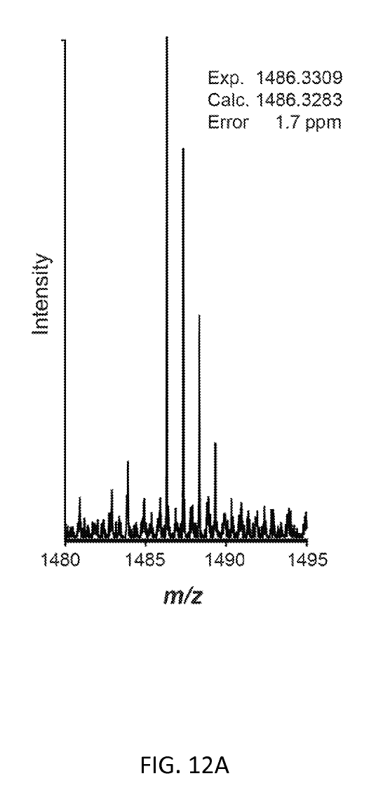

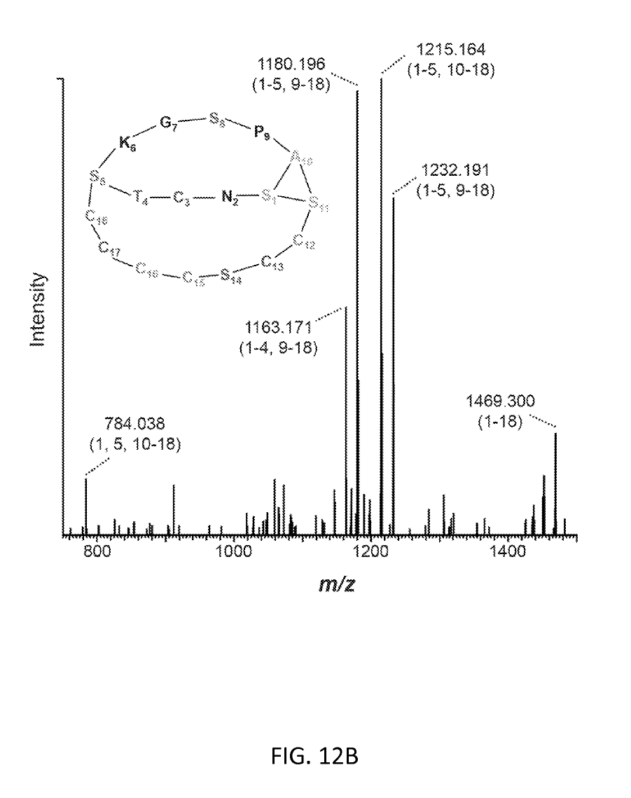

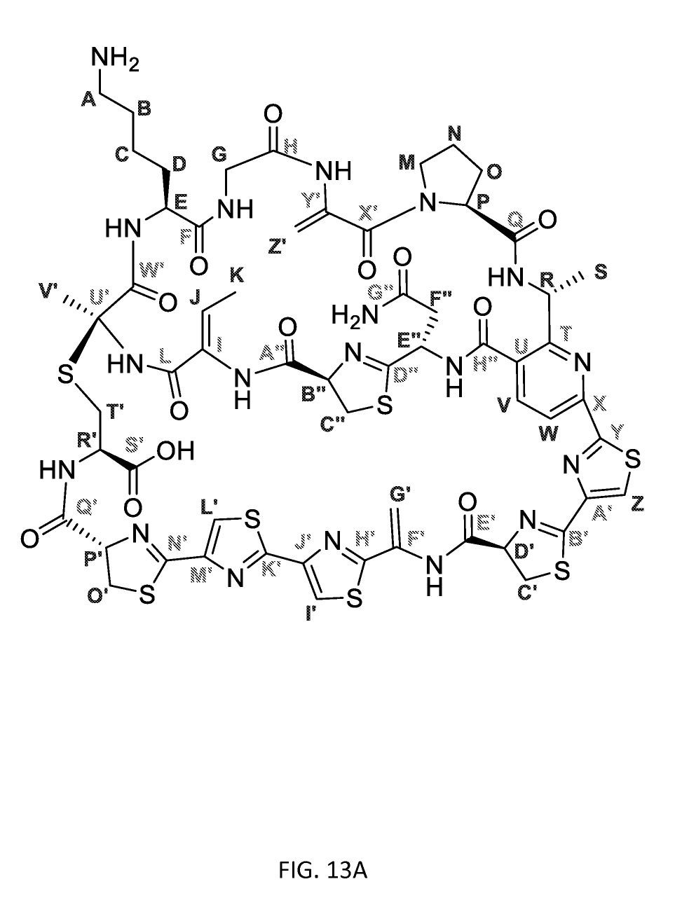

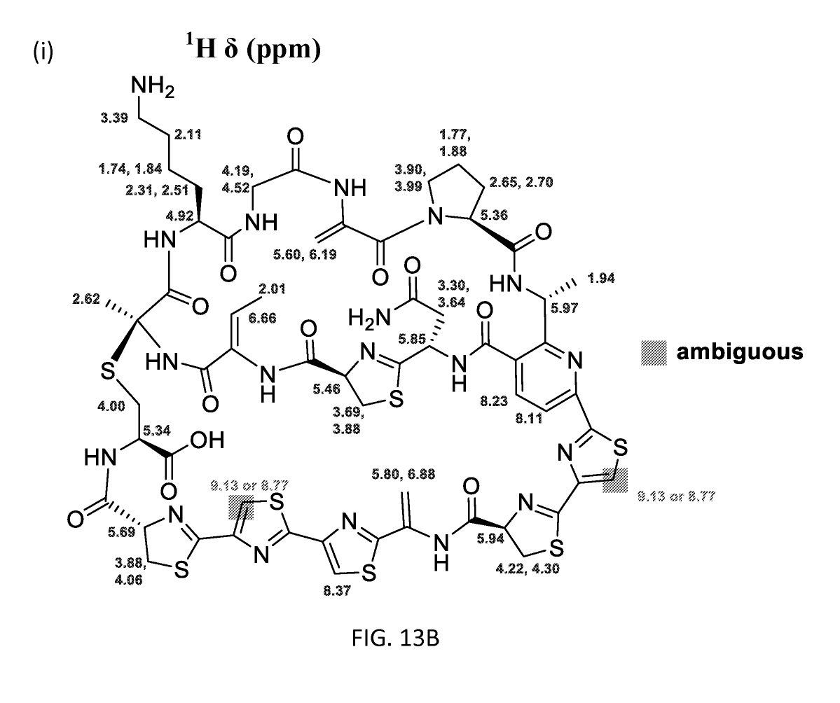

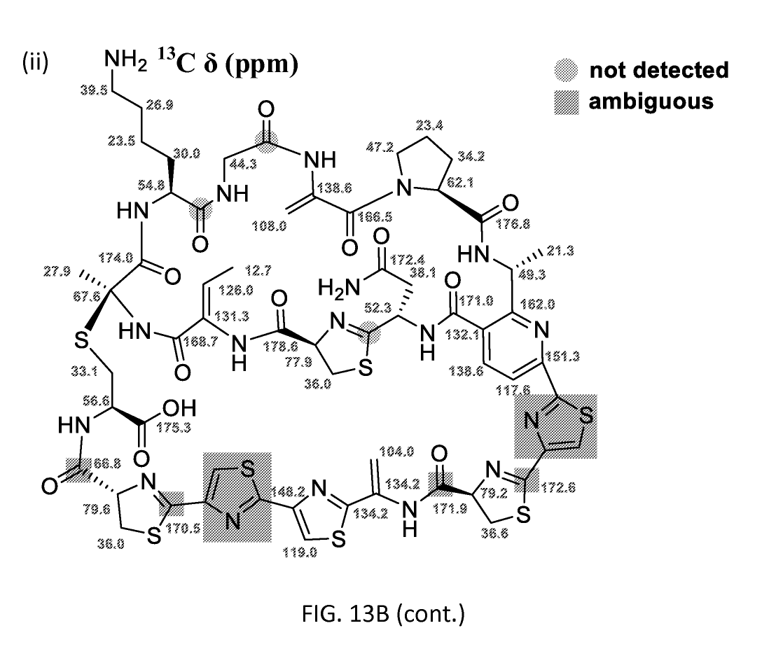

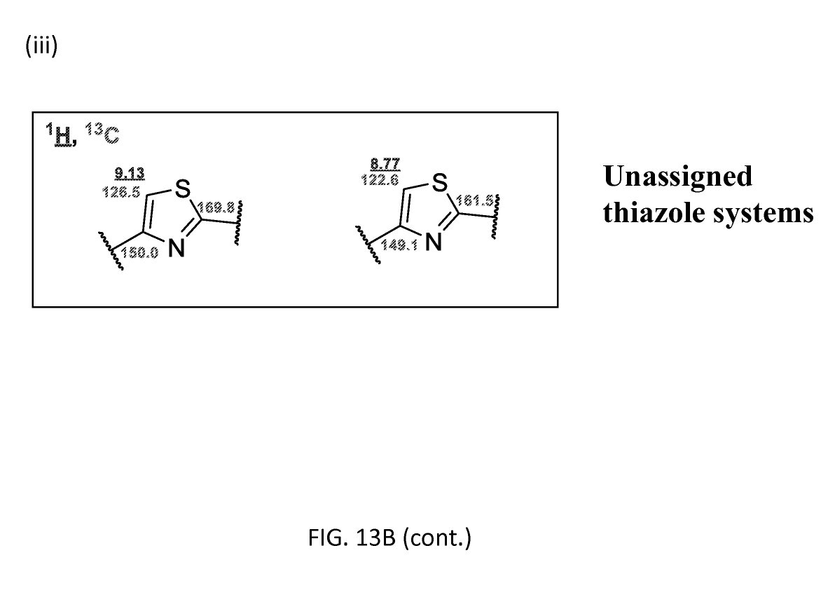

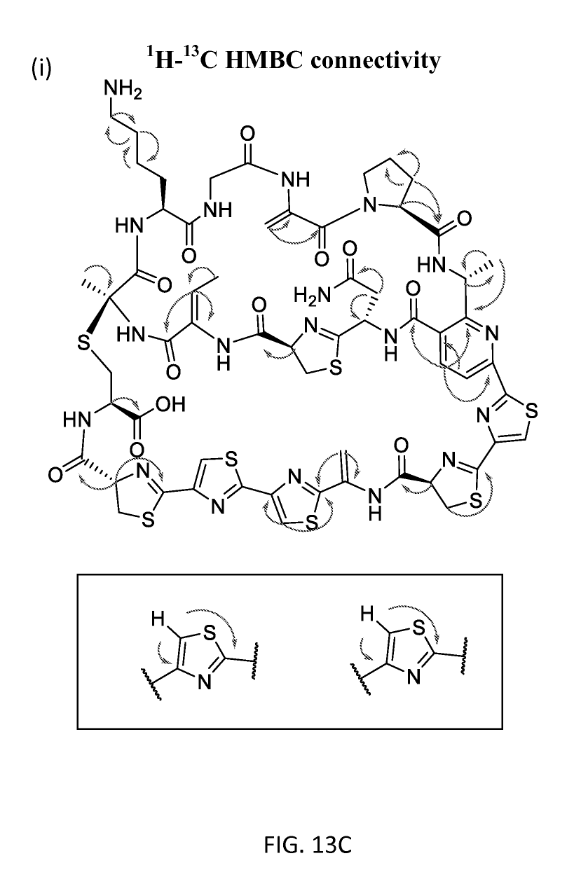

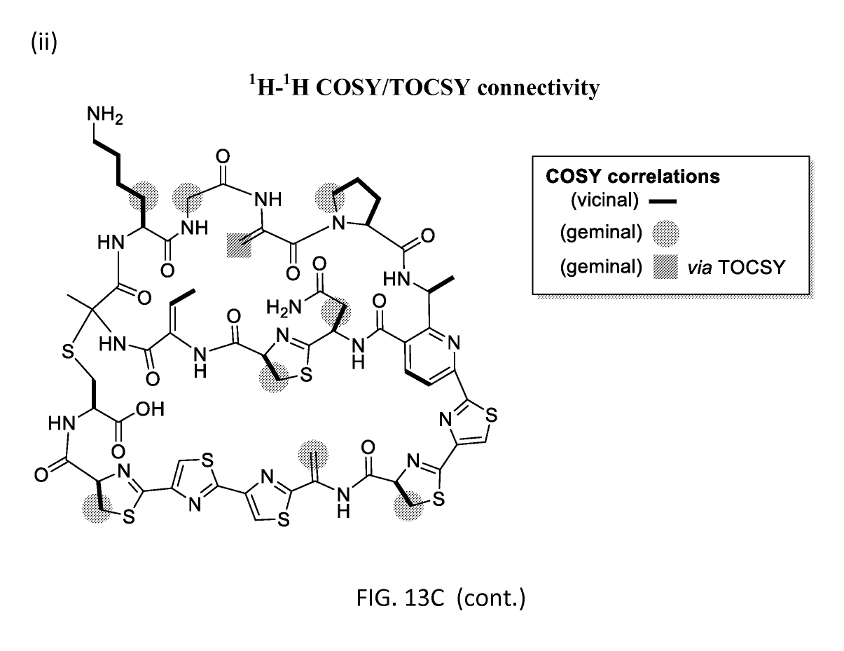

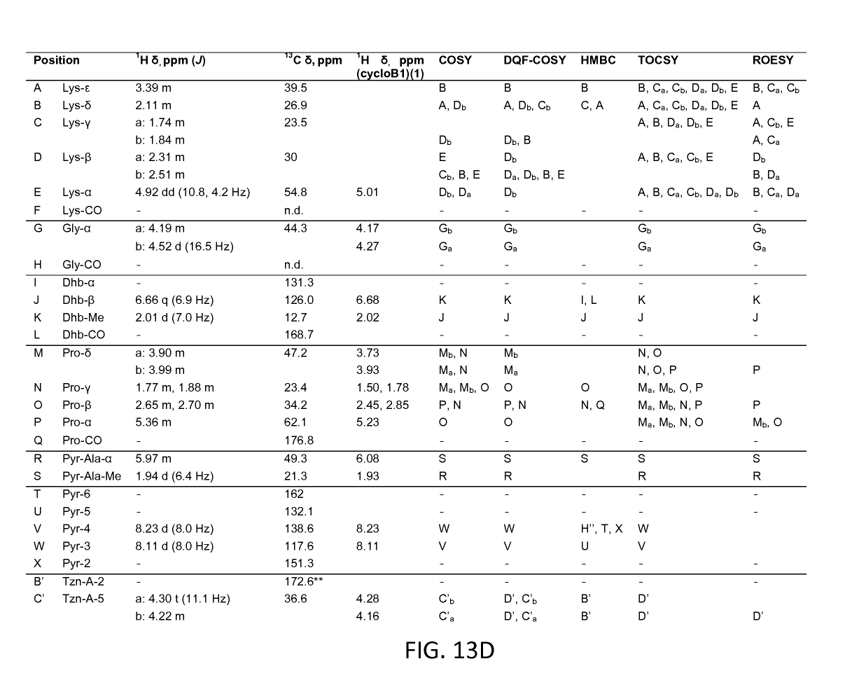

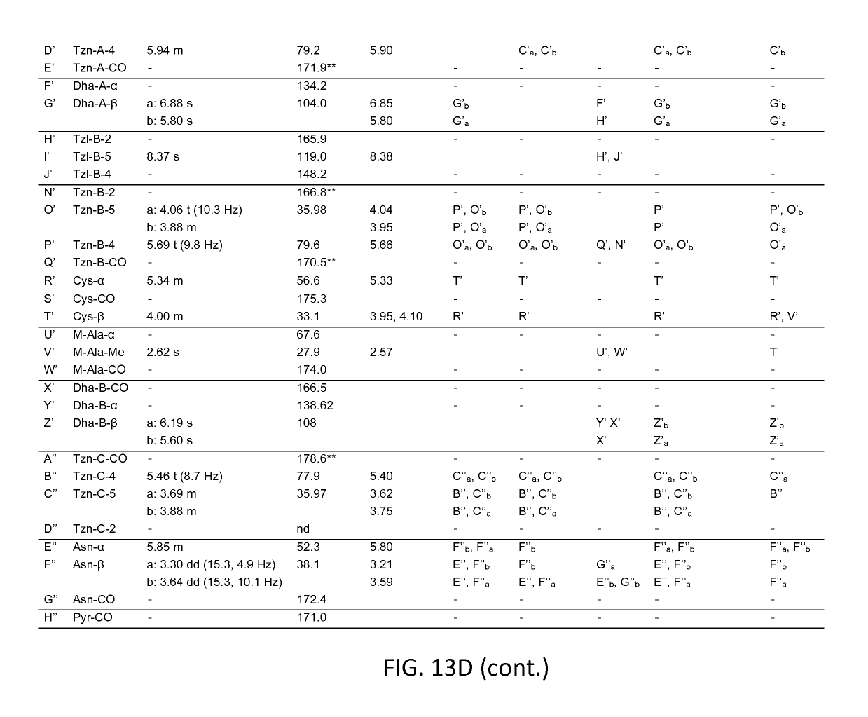

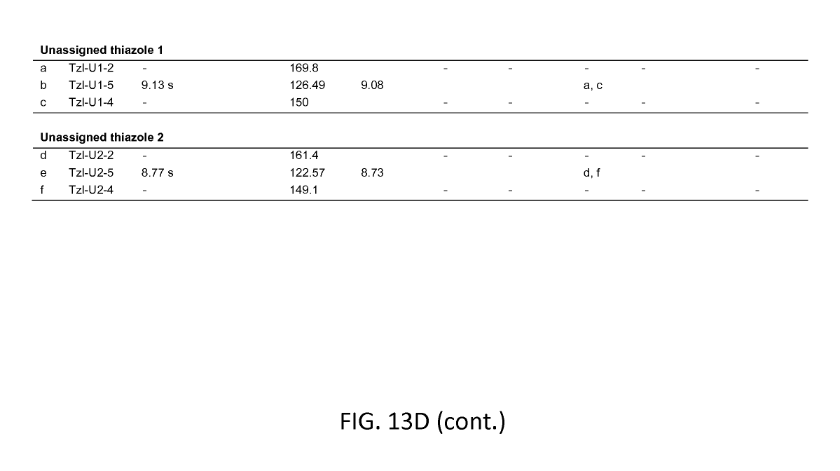

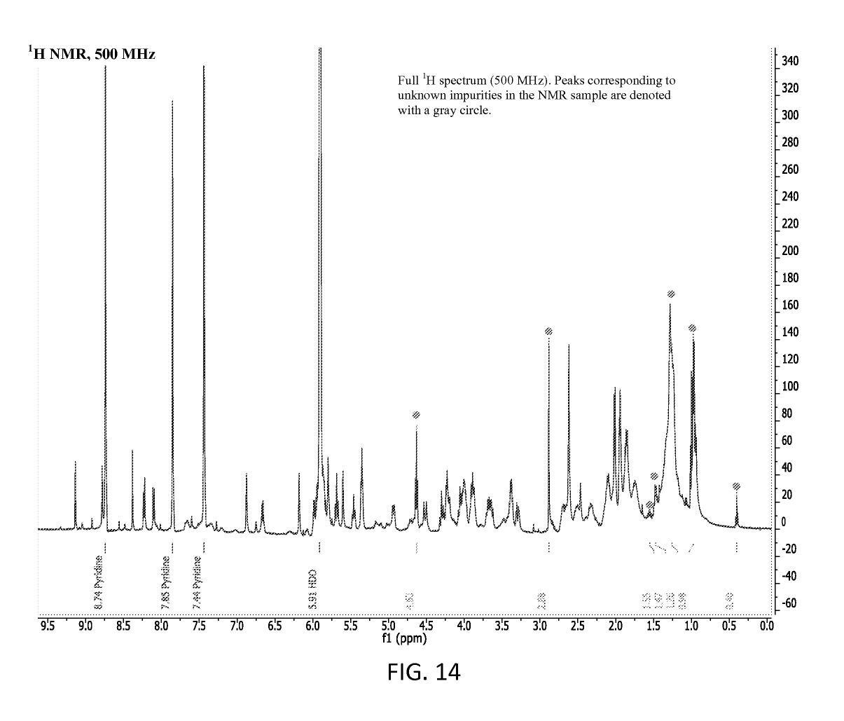

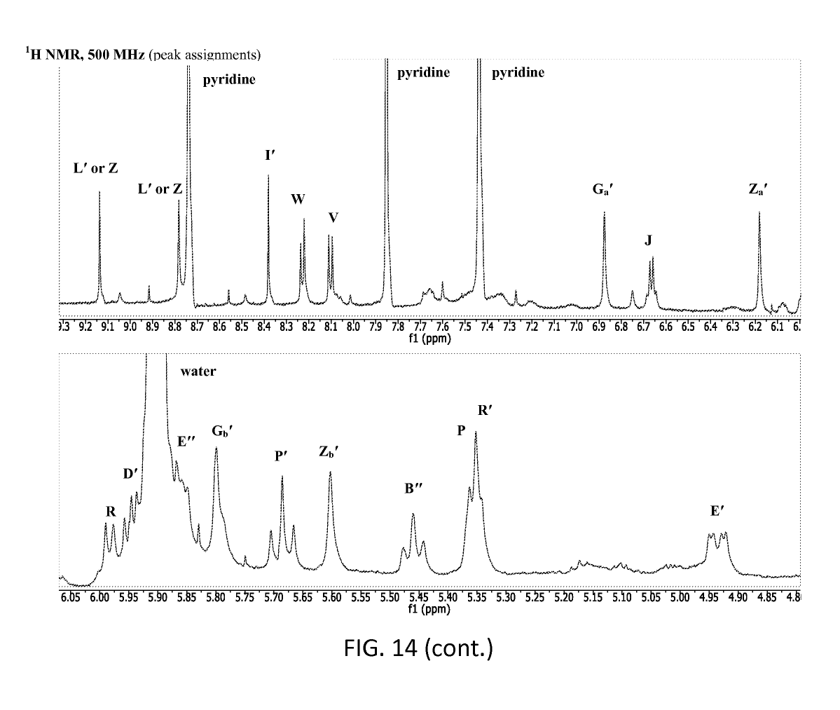

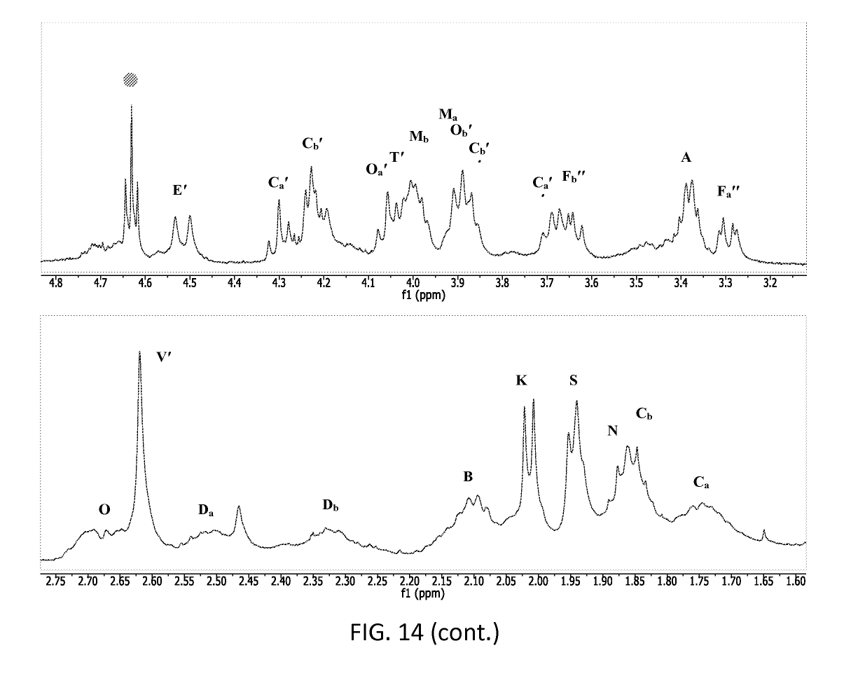

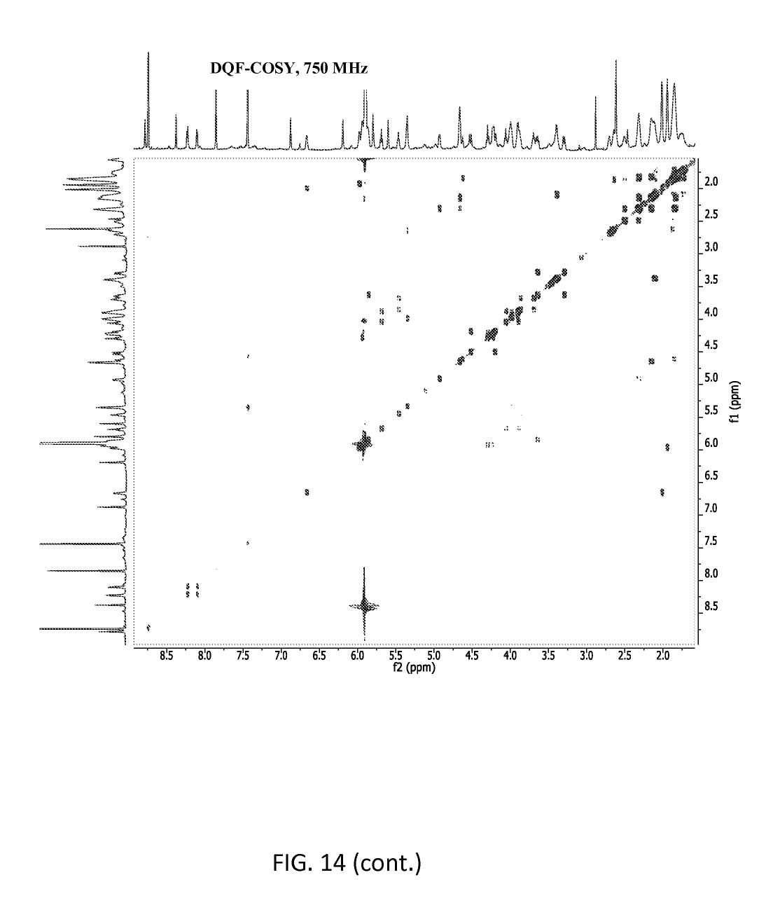

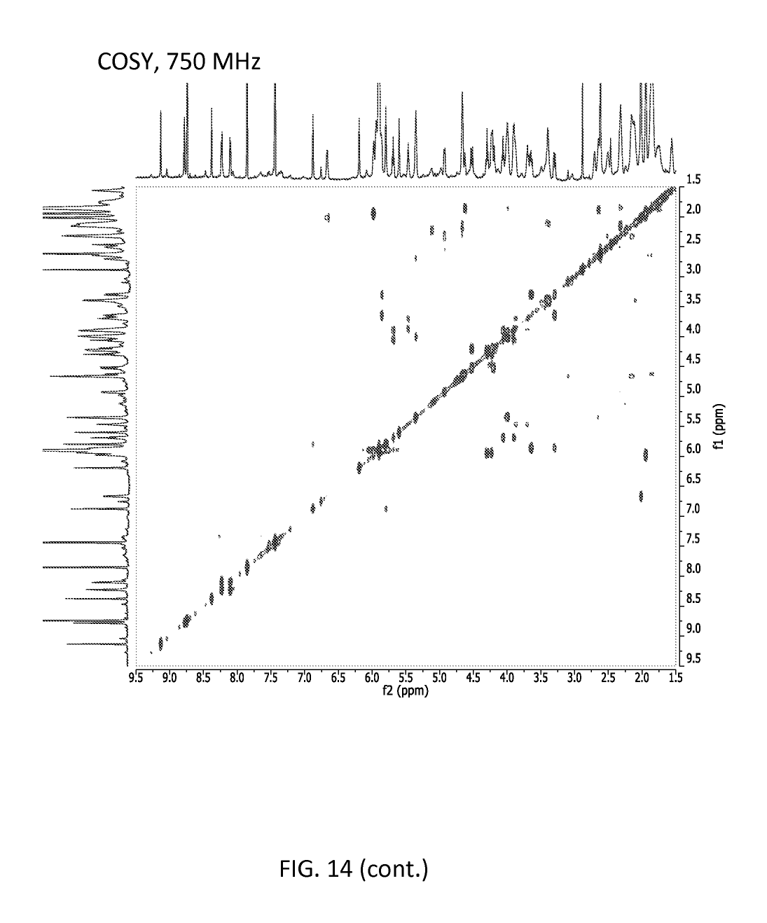

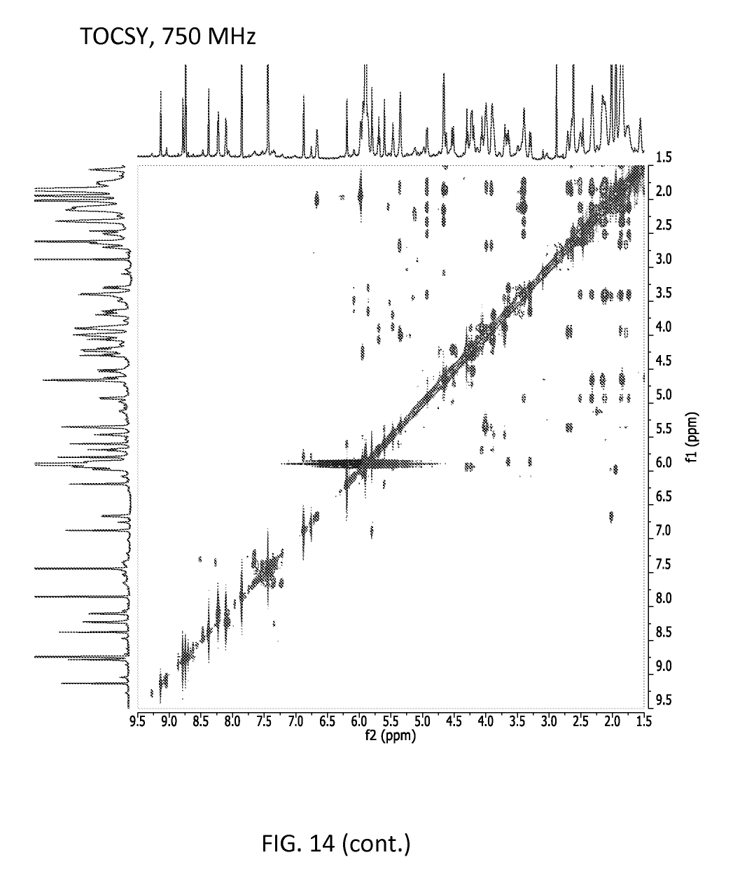

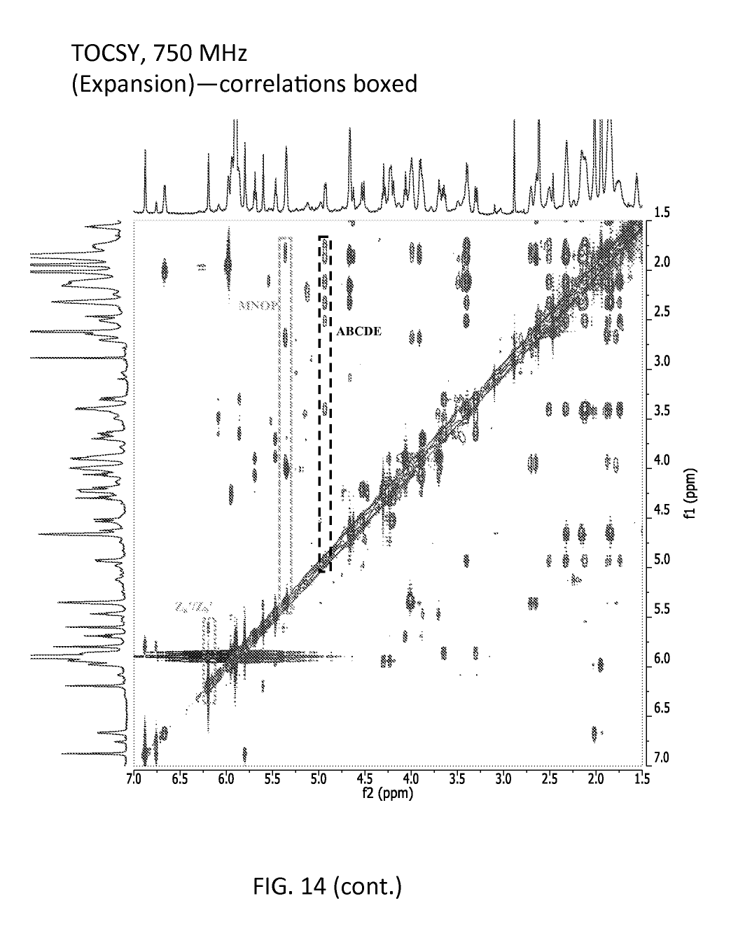

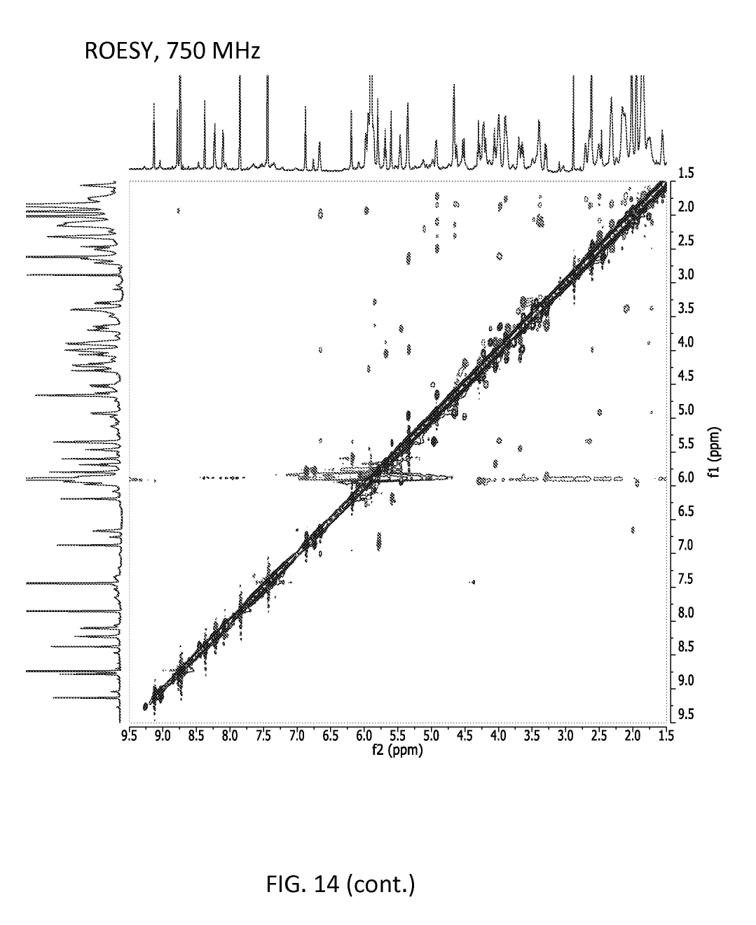

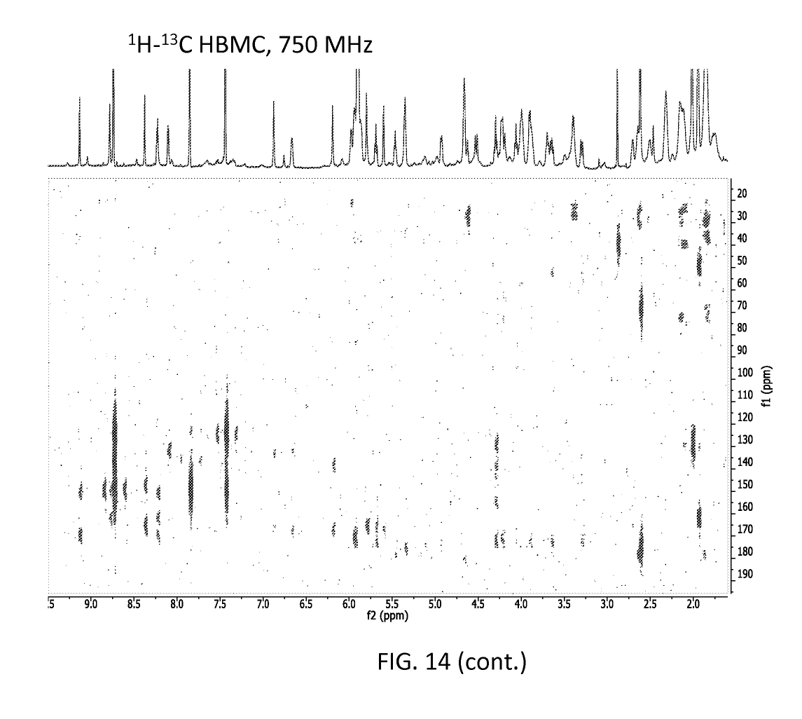

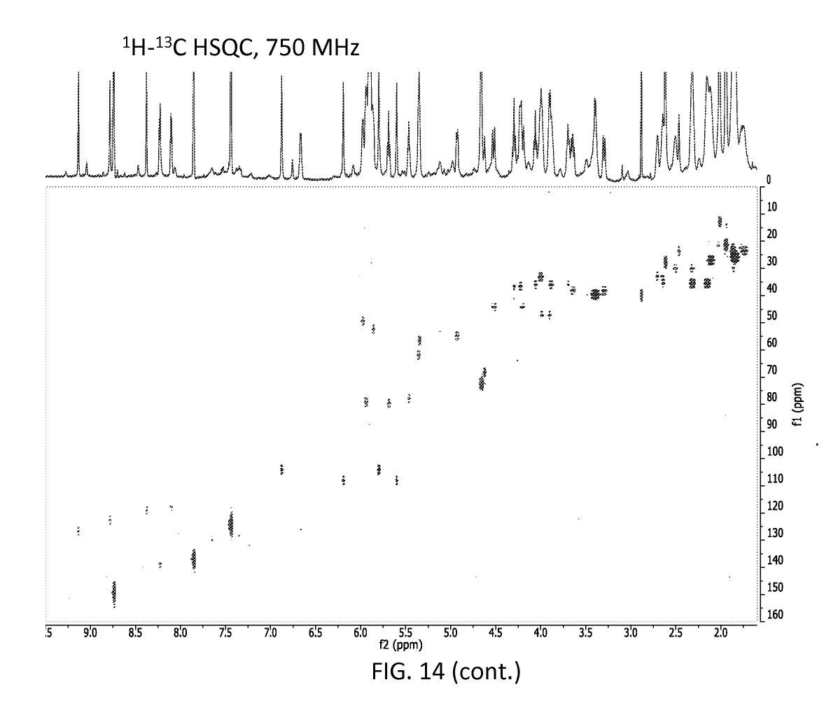

Prior to detailed structural characterization, cyclothiazomycin C was purified by MPLC and HPLC (FIG. 11). The mass spectrum of purified cyclothiazomycin C revealed an [M+H]⁺ ion of m/z 1486.3309 Da (FIG. 12A), supporting the molecular formula for the predicted structure of cyclothiazomycin C (C₆₀H₆₇N₁₉O₁₃S₇). Analysis of the collision-induced dissociation (CID) mass spectrum corroborated the amino acid sequence of the precursor peptide, strongly connecting the predicted gene cluster to the mature natural product (FIG. 12B). NMR spectroscopy was then used to confirm the predicted structure of cyclothiazomycin C (FIGS. 13, 14). Bond connectivity was established using ¹H—¹H COSY, ¹H—¹H TOCSY, ¹H—¹³C HSQC, and ¹H—¹³C HMBC experiments. Chemical shifts were assigned from this information and by comparison to the known values for cyclothiazomycin B. Due to the spectral similarity to cyclothiazomycin B, we have assigned the stereochemistry of cyclothiazomycin C analogously to the reported compound.

### Conservation Analysis of the Cyclothiazomycin C Biosynthetic Gene Cluster

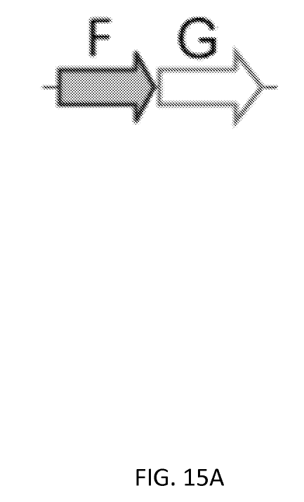

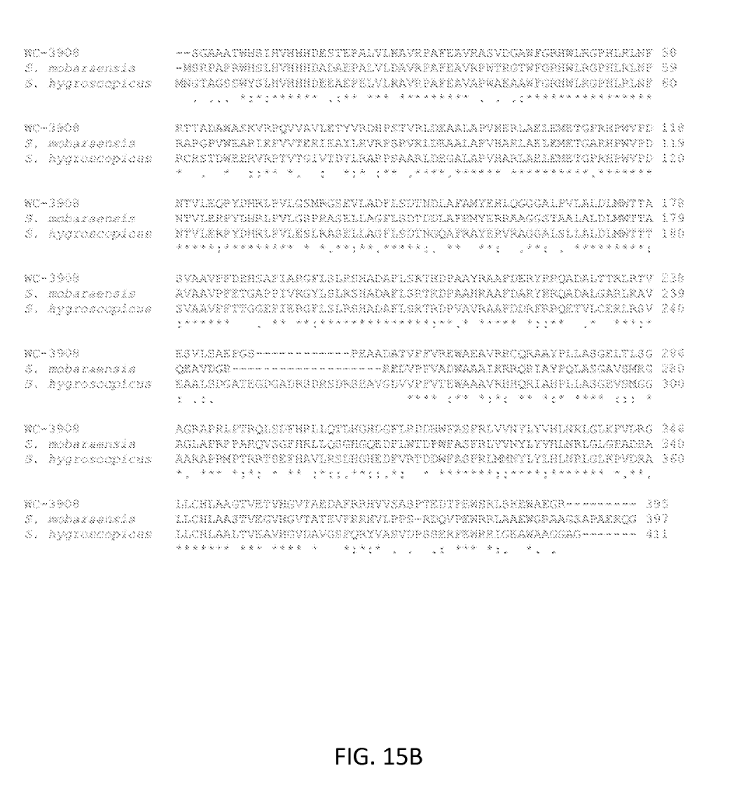

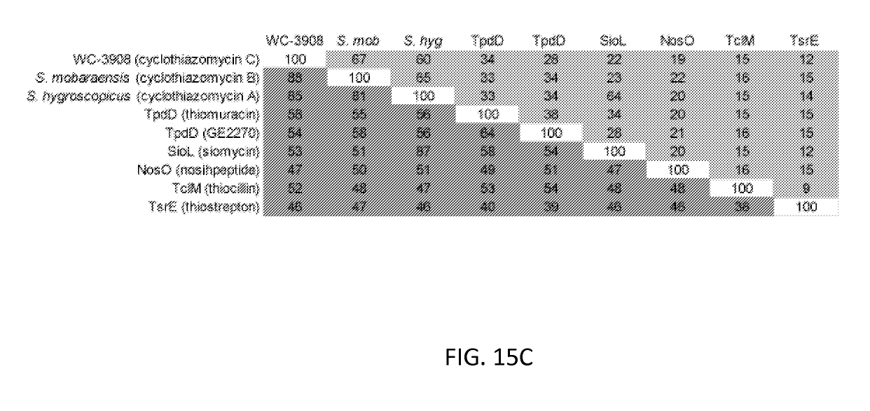

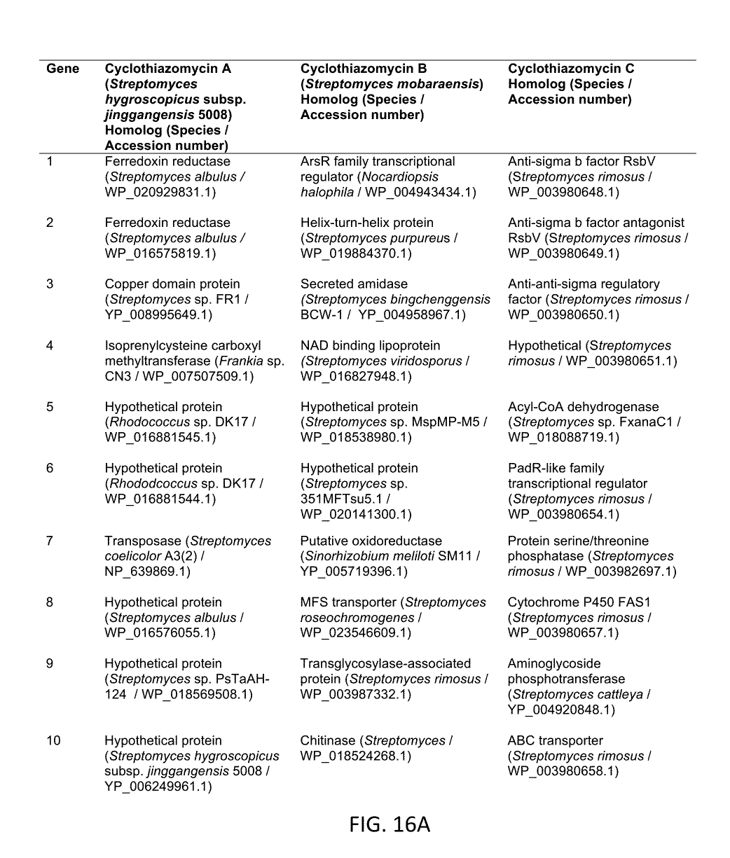

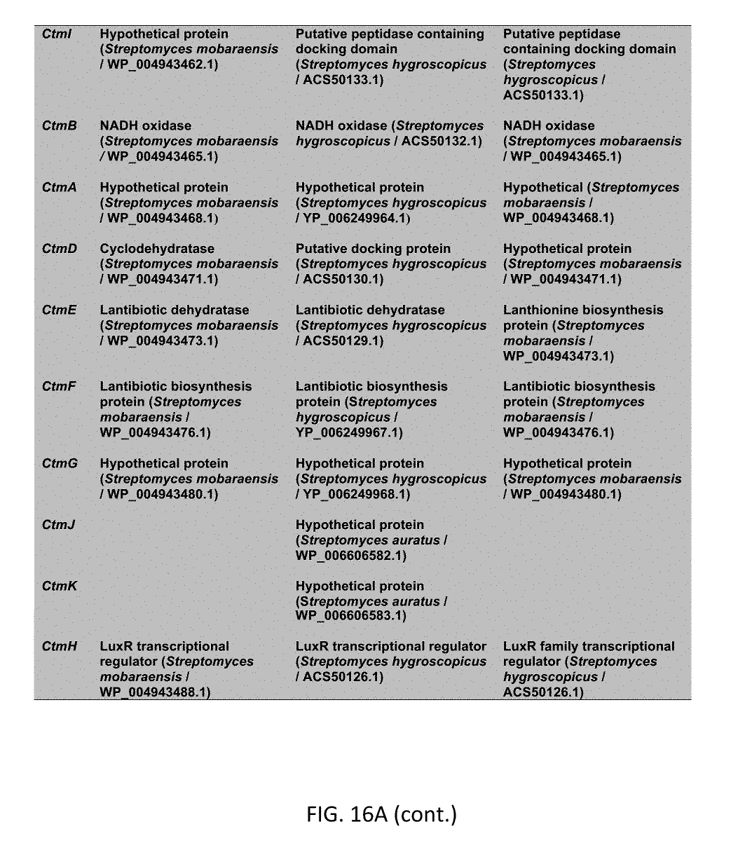

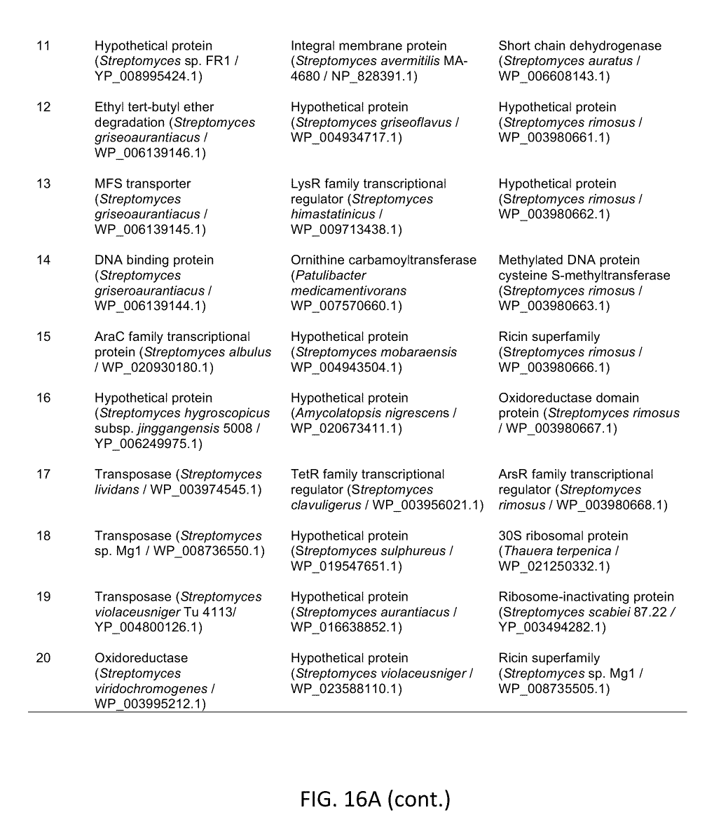

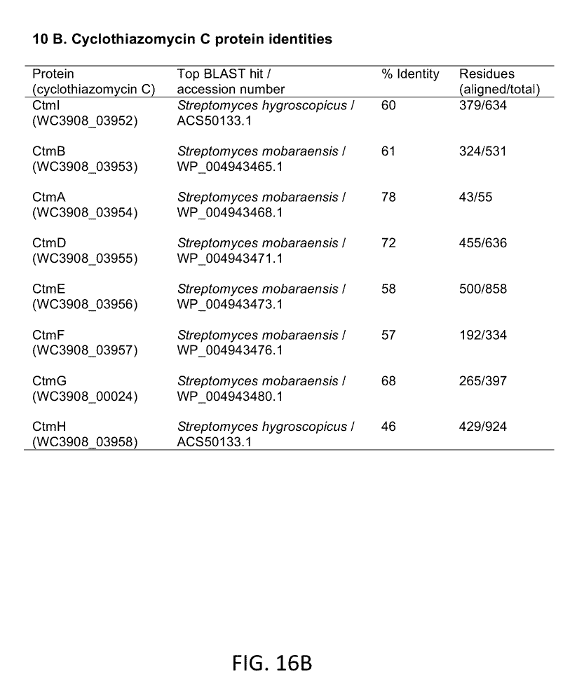

To provide additional evidence that the thiopeptide gene cluster from WC-3908 was responsible for the production of cyclothiazomycin C, conservation analysis was performed with the cyclothiazomycin A, B, and C (putative) gene clusters. The cyclothiazomycin A biosynthetic genes derived from Streptomyces hygroscopicus subsp. jinggangensis 5008 while the cyclothiazomycin B genes were from Streptomyces mobaraensis. A subset of the genes predicted for the production of cyclothiazomycin B was conserved among the three clusters (FIG. 10B). All three clusters contain a short open reading frame, here designated ctmA, encoding the precursor peptide. CtmD encodes a “fused” TOMM cyclodehydratase (E1 ubiquitin-activating enzyme/MccB-like and YcaO domains), which implicates CtmD in the formation of thiazolines. CtmB encodes a flavin mononucleotide-dependent protein, putatively responsible for the dehydrogenation of the thiazolines to thiazoles. CtmE and ctmF encode homologs of a split lanthipeptide dehydratase, which performs the dehydration of serine and threonine to dehydroalanine and dehydrobutyrine. Like all thiopeptides, cyclothiazomycin C has a substituted 6-membered, nitrogen-containing central heterocycle (here a pyridine). In the case of cyclothiazomycins A and B, the pyridine moiety is likely formed by the gene product of ctmG, given the homology to tclM, which has been implicated in the formal [4+2] cycloaddition reaction during thiocillin biosynthesis (FIG. 15). For cyclothiazomycin C, a gene with high similarity to ctmG from the cyclothiazomycin A and B clusters is present, but distantly located in the genome, indicating that the cyclothiazomycin C gene cluster is fragmented. Interestingly, ctmG from WC-3908 is found directly next to a gene duplication of ctmF, which is suggestive of paralogous duplication (FIG. 15). CtmI, which is present in all three clusters, encodes a ThiF-like protein. ThiF-like proteins have been implicated in the biosynthesis of thiamine diphosphate in E. coli. However, the function of ThiF-like proteins in the context of TOMM biosynthesis remains to be established. Other local genes include ctmH, which is a LuxR-type regulatory gene and ctmJK, which are omitted from the cyclothiazomycin A and C clusters and have no known function (FIG. 10). We further note that the genes flanking the conserved region are highly disparate between the three clusters (FIG. 16). This subset of genes, ctmA-G and ctml from Streptomyces hygroscopicus subsp. jinggangensis 5008 were recently shown to be regulated by the LuxR-type regulatory gene ctmH. Furthermore, the deletion of ctmA, ctmD, ctmF, and ctmG abolished the production of cyclothiazomycin A. These data further support the gene cluster prediction for cyclothiazomycin C from WC-3908.

### Assessment of Cyclothiazomycin Bioactivity

Previous reports on cyclothiazomycins A and B describe a wide range of bioactivities, including renin inhibition, RNA polymerase inhibition, and antifungal activity. We found that purified cyclothiazomycin C exhibited growth inhibitory action toward several Gram-positive (Firmicutes) bacteria but was inactive against all tested Gram-negative (Proteobacteria) organisms (Table 6).

Antimicrobial activity of cyclothiazomycin B and C toward a
panel of diverse bacteria and fungi.

| | MIC^{b}, | MIC^{b}, |
|---|---|---|
| Species^{a} | cyclothiazomycin B | cyclothiazomycin C |
| Bacillus anthracis | 1 | 1 |
| Bacillus subtilis | 2 | 4 |
| Enterococcus faecalis | 32 | 32-64 |
| Listeria monocytogenes | 8 | 16 |
| Staphylococcus aureus | 4 | 16 |
| Escherichia coli | 64 | >64 |
| Neisseria sicca | >64 | >64 |
| Pseudomonas putida | >64 | >64 |
| Aspergillus niger | >64 | >64 |
| Fusarium virguliforme | 64 | >64 |
| Saccharomyces cerevisiae | 64 | >64 |
| Talaromyces stipitatus | 64 | >64 |

^{a}The top five species are Gram positive bacteria from the Firmicutes phylum. The next three species are Gram negative bacteria from the Proteobacteria phylum. The lowest 4 species are fungi from the Ascomycota phylum.
^{b}All minimum inhibitory concentrations (MIC) were determined by the microbroth dilution method and are presented in μg/mL.

The greatest inhibitory activity was observed towards the genus Bacillus. We decided to also evaluate if cyclothiazomycin C exhibited growth inhibitory action toward a variety of fungal strains, but none was observed.

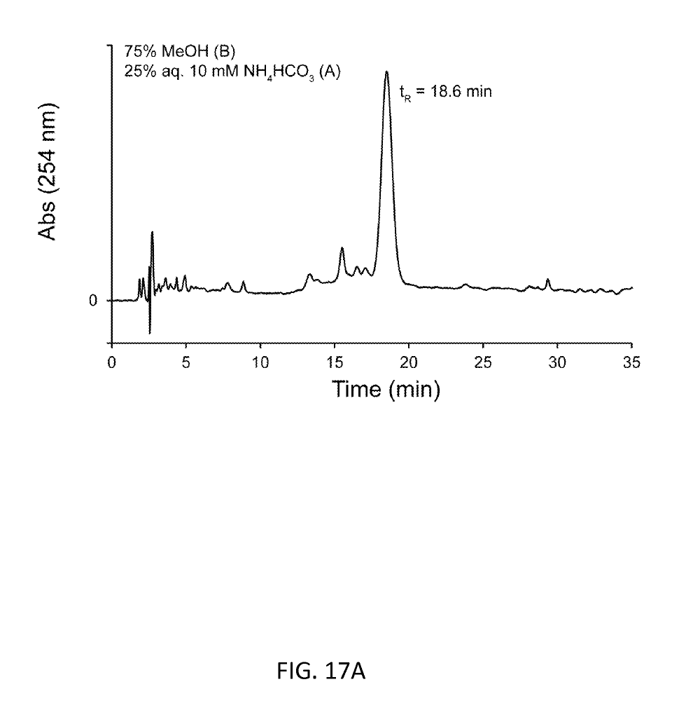

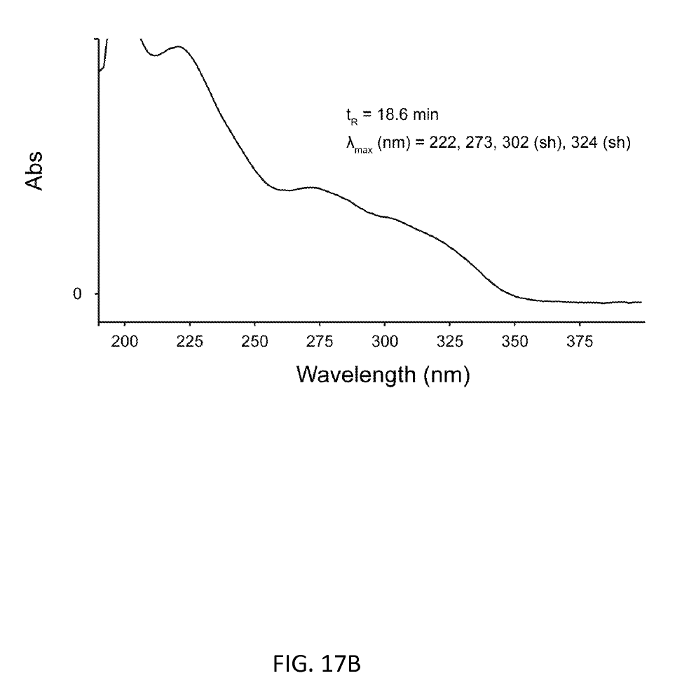

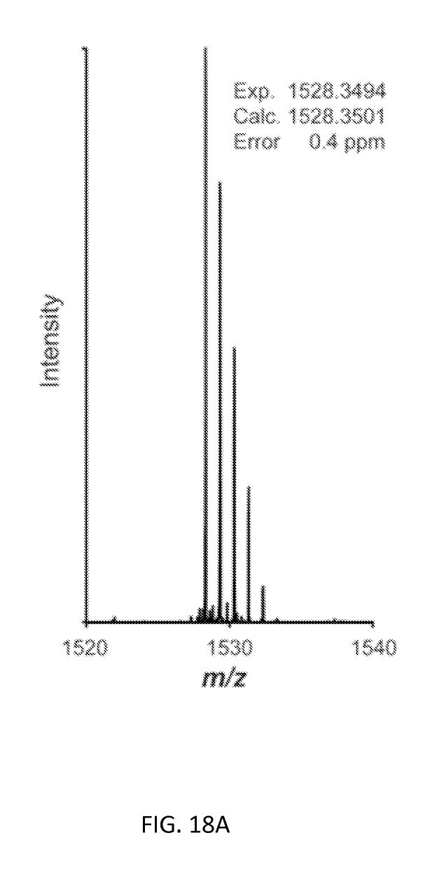

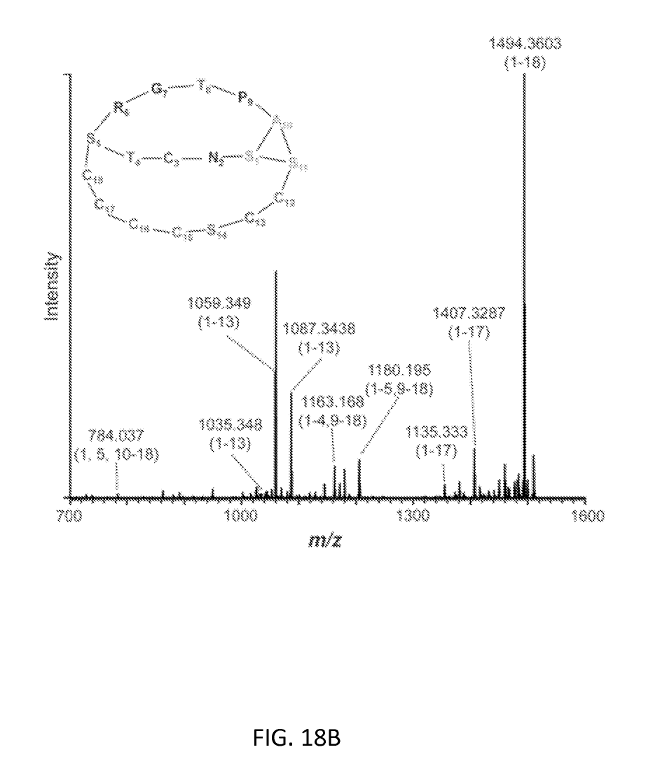

To further clarify cyclothiazomycin bioactivity, we obtained a cyclothiazomycin B producer, strain with the NRRL identifier B-3306, and purified cyclothiazomycin B in a manner analogous to that employed for cyclothiazomycin C (FIGS. 17, 18). As above, we assessed cyclothiazomycin B for antibiotic and antifungal activity. Cyclothiazomycin B also had the greatest inhibitory activity towards the genus Bacillus, with little to no activity against a panel of Gram-negatives and fungal strains. This activity does not align with previous reports (Hashimoto, M. et al. (2006) “An RNA polymerase inhibitor, cyclothiazomycin B1, and its isomer,” Bioorg. Med. Chem. 14, 8259-8270; Mizuhara, N., Kuroda, M., Ogita, A., Tanaka, T., Usuki, Y., Fujita, K. (2011) Antifungal thiopeptide cyclothiazomycin B1 exhibits growth inhibition accompanying morphological changes via binding to fungal cell wall chitin, Bioorg. Med. Chem. 19, 5300-5310); however, additional fungal strains will need to be tested to more concretely establish cyclothiazomycin spectrum of activity. The antibiotic activity of cyclothiazomycin B and C are similar to known thiopeptides, which act as translation inhibitors by binding to either the 50S subunit or EF-Tu. It is possible that the cyclothiazomycins act in a similar manner but the determination of the precise mode of action will require further exploration.

### Growth Conditions And High Throughput Culture Preparations for Screening

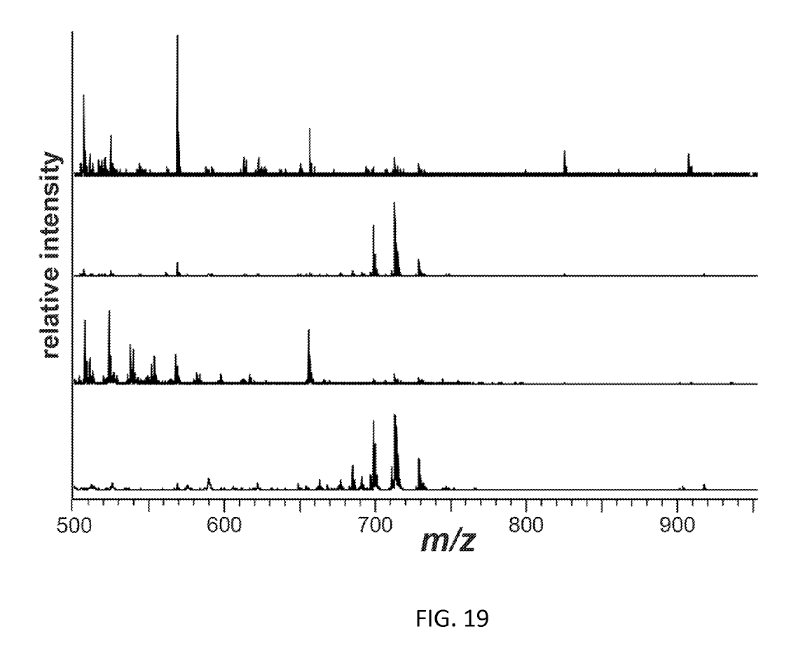

The artificial conditions used to cultivate bacteria do not accurately depict the variable nutritional and stress environments encountered in nature; thus, many biosynthetic gene clusters are transcriptionally silent. Certain culture additives can stimulate NP biosynthesis to a level that allows for a full structural and functional characterization. Well known additives for this aspect include γ-butyrolactones, DMSO, GlcNAc, and sublethal concentrations of antibiotics, especially trimethoprim. Additionally, some NP biosynthetic pathways are stimulated in nutrient poor media, while others require a richer medium, either in solid or liquid formats. In addition to the previously mentioned additives, certain distinct media can produce unique NP profiles (FIG. 19). For a group of actinomycetes bioinformatically predicted to produce the same NP, cultivation in only four media led to detection of the desired NP in 25% of the predicted producers. Thus, the methods disclosed here can be used in combination with a variety of growth conditions on distinct strains predicted to produce identical NPs.

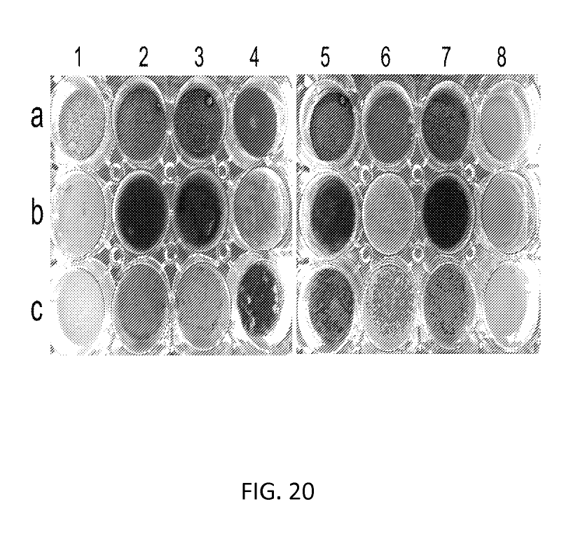

Actinomycetes vastly change their secondary metabolism upon switching from liquid to solid agar media. Though other plate formats are amenable for use in this application, previous experience informs us that 12-well plates (2 mL agar per well) can give a sufficient balance of culture size and throughput for our purpose (FIG. 20). The above-mentioned 192 actinomycetes on 16×12-well plates can be grown in a rich, agar-containing medium. As with the liquid format, these plates can serve to inoculate future cultures for one type of reactivity probe-based screening method (for example, 1,4-nucleophilic addition-based screening) while other plates will be prepared for strains to be screened by additional NP discovery reactions (see, for example, exemplary reagents and reactions outlined in Tables 1-5). The 12-well plates are also reusable, reducing one's waste stream.

One disadvantage to the above set up is that upon inoculation there is a non-zero probability of strain cross-contamination. To reduce cross-contamination, one can prepare in advance several hundred 12-well plates that contain 3 or 4 different media conditions (these can be stored sterile for ˜4-6 months at 4° C.). Three or four distinct actinomycete strains can then be grown per plate, reducing the probability of cross-contamination. In deciding which strains to co-populate a single plate, the criterion will be that the strains are predicted to produce identical (or nearly so) NPs. This way, if cross-contamination occurs, we do not lose our bioinformatic link to the genome that facilitates structure determination. In case one needs to further explore a set of strains, the advanced preparation of variable media in 12-well format allows one the flexibility to rapidly assess the effects of culture additives. For instance, GlcNAc, trimethoprim, and a γ-butyrolactone could be soaked into the agar on successive rows, yielding a single plate 12 unique growth conditions.

### SUMMARY

A new reactivity-based screening method is disclosed to conveniently identify any type of natural product that bears the organic functional group undergoing derivatization. This method employs ubiquitous reagents and instrumentation, making it a broadly accessible strategy for natural product discovery. Three characteristics make the labeling procedures operationally straightforward: (a) anhydrous solvents are unnecessary, meaning the reaction is performed under ambient atmosphere; (b) the reagents employed are common in most laboratories and easily handled; and (c) the large excess of labeling reagent relative to the substrate means that precise stoichiometric calculations for each reaction are unnecessary. Although under these excess labeling conditions, minor peaks related to non-target specific labeling are observed often, these species never convoluted spectral interpretation. When compared to traditional bioassay-guided isolation strategies, which can require many thousands of samples to be screened to discover new compounds, the method's discovery rate highlights the efficiency of this tandem strategy. Further, the compound(s) to be discovered do not need to be present at bioactive concentrations, but merely need to be detectable upon labeling, which capitalizes on the remarkable sensitivity of mass spectrometry. With the substantial rise of available genomic sequences, the combination of bioinformatics and simple chemoselective reactivity-based labeling will provide a powerful tool to identify novel natural products, while dramatically reducing the time invested on the unfruitful rediscovery of known compounds.

### Utility and Applications

In a first aspect, a method of identifying a natural product including NP—[X]ₙ is disclosed. The method includes several steps. The first step includes selecting an organism having a biosynthetic pathway for producing the natural product including NP—[X]ₙ using a bioinformatics algorithm. The second step includes preparing a sample suspected to contain NP—[X]ₙ comprising a complex cellular metabolite mixture from an organism. The third step includes reacting the sample suspected to contain NP—[X]ₙ with reactivity probe Y according to Scheme I:

NP—[X]ₙ+Y→NP—[X]ₙ₋ₘ[Z]ₘ Scheme I.

NP—[X]ₙ represents a natural product NP having a chemical moiety X that is susceptible to chemical modification by reactivity probe Y to form at least one product adduct NP—[X]ₙ₋ₘ[Z]ₘ in which chemical moiety X reacts with reactivity probe Y to form adduct Z, wherein n ranges from 1 to about 10 and m is at least 1 and m≦n. The fourth step, which is optional in some cases, includes dereplicating the product collection of at least one known labeled metabolite to provide a depleted product collection including at least one unknown labeled metabolite. The fifth step includes determining the structure of the at least one unknown labeled metabolite, thereby identifying the natural product including NP—[X]ₙ.

In one aspect, the bioinformatics algorithm includes several steps. The first step includes populating a list of strains encoding a first biosynthetic enzyme. The second step includes reducing the list of strains encoding a second biosynthetic enzyme to yield a refined list of strains, wherein the second biosynthetic enzyme is encoded by a gene within a range of ten open reading frames of a gene encoding the first biosynthetic enzyme. The third step includes identifying precursor peptide products of the first biosynthetic enzyme from the refined list of strains. Both the first and second biosynthetic enzymes catalyze transformations in the biosynthetic pathway for producing the natural product including NP—[X]ₙ.

In a refinement of this aspect, the first biosynthetic enzyme includes a thiazole/oxazole-modified microcin (TOMM) cyclodehydratase and the second biosynthetic enzyme includes a lantibiotic dehydratase, and chemical moiety X is a dehydrated amino acid.

In one aspect, the step of dereplicating the product collection of at least one known labeled metabolite includes two steps. The first step includes identifying the presence in the product collection including labeled metabolites the at least one known labeled metabolite having a mass of a labeled natural product predicted from a precursor peptide product from the organism selected using the bioinformatics algorithm. The second step includes removing the at least one known labeled metabolite from further characterization.

In a refinement of this aspect, the step of identifying the presence in the product collection including labeled metabolites the at least one known labeled metabolite includes applying differential mass spectrometry to characterize the at least one known labeled metabolite.

In one aspect, the step of dereplicating the product collection of at least one known labeled metabolite includes applying differential mass spectrometry to characterize the product collection.

In one aspect, the organism is a bacterium or a fungus.

In one aspect, reactivity probe Y has the structure of Formula (I):

R-L-Q (I),

wherein R is a reactive moiety that reacts with chemical moiety X, L is a linker and Q is a label.

In one refinement of this aspect, the label Q is selected from an affinity label, a detectable group and a physicochemical label. In one aspect, label Q includes an affinity probe. In one refinement of this aspect, the affinity probe is selected from biotin, streptavidin, polyhistine (for example, (His₆)), an unreacted thiol group of dithiothreitol, glutathione-S-transferase (GST), HaloTag®, AviTag, Calmodulin-tag, polyglutamate tag, FLAG-tag, HA-tag, Myc-tag, S-tag, SBP-tag, Softag 3, V5 tag, Xpress tag, and a hapten. In a further refinement of this aspect, the affinity probe includes Formula (A):

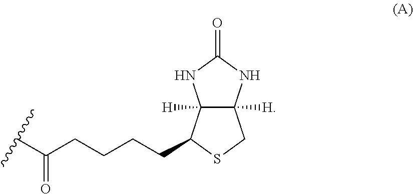

In another refinement, label Q includes a detectable group. In one refinement of this aspect, detectable group is selected from a radiolabel, a fluorescent label, and a chemiluminescent label. In another refinement of this aspect, the detectable group includes a fluorescent label. In yet a further refinement of this aspect, the fluorescent label includes Formula (B):

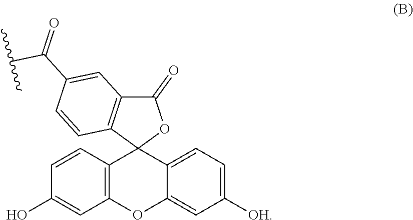 [SMILES: *C(=O)c1ccc2c(c1)C(=O)OC21c2ccc(O)cc2Oc2cc(O)ccc21]

In another refinement, label Q includes a physicochemical label. In one refinement of this aspect, the physicochemical label is selected from an isotopic label and a mass label. In a further refinement of this aspect, the physicochemical label includes a cation mass label. In yet a further refinement of this aspect, the cation mass label includes Formula (C):

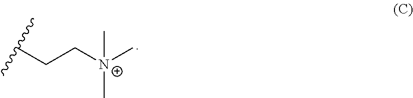 [SMILES: *CC[N+](C)(C)C]

In one aspect, label Q is selected from the following:

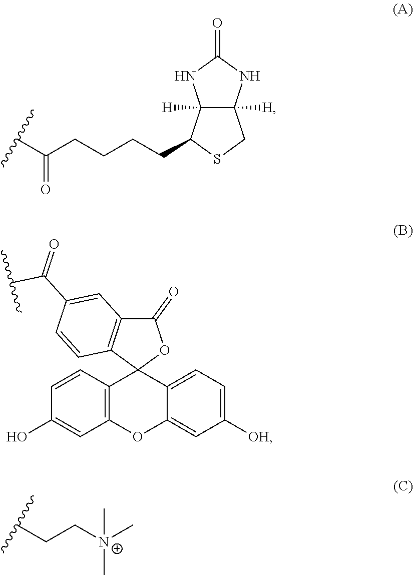

and combinations thereof.

In one aspect, reactivity probe Y is selected from the following:

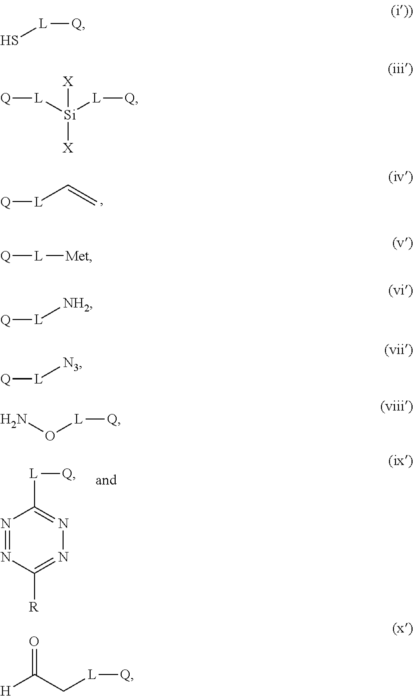

or a combination thereof,
wherein R is alkyl or L-Q.

In one aspect, reactivity probe Y is selected from an aminooxy-based reactivity probe, an aldehyde-based reactivity probe, a thiol-based reactivity probe and a tetrazine-based reactivity probe, or a combination thereof.

In another aspect, reactivity probe Y comprises an aminooxy-based reactivity probe. In this aspect, the aminooxy-based reactivity probe is selected from

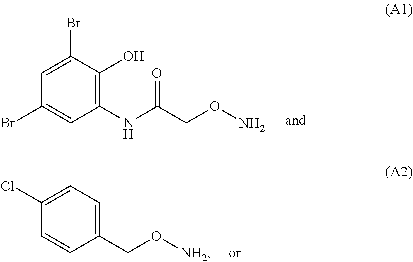 [SMILES: NOCC(=O)Nc1cc(Br)cc(Br)c1O ; NOCc1ccc(Cl)cc1]

a combination thereof.

In another aspect, reactivity probe Y comprises an aldehyde-based reactivity probe. In this aspect, the aldehyde-based reactivity probe is

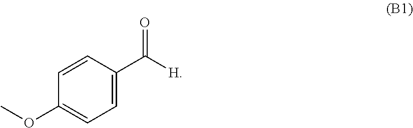

In another aspect, reactivity probe Y comprises a thiol-based reactivity probe. In this aspect, the thiol-based reactivity probe is selected from

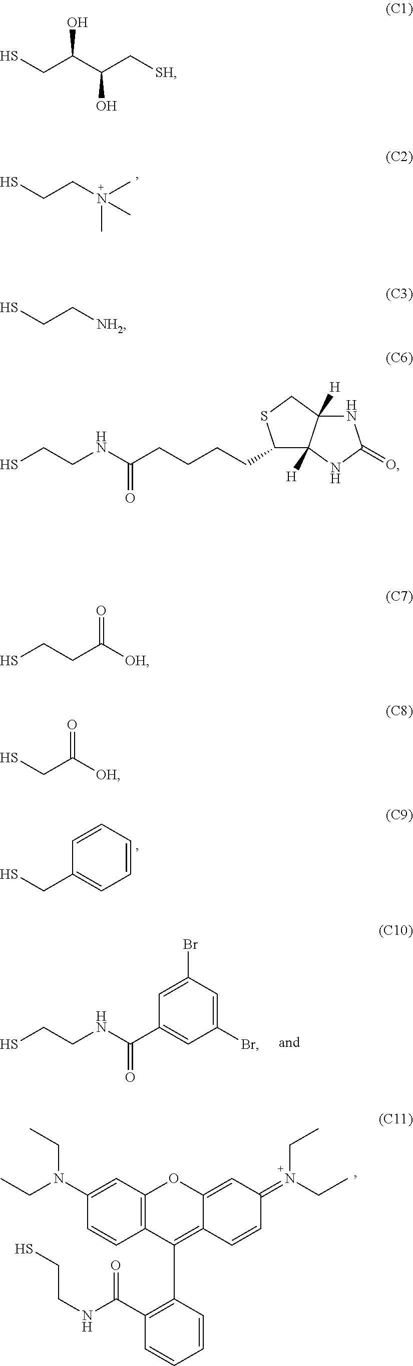

or a combination thereof.

In one aspect, reactivity probe Y comprises a tetrazine-based reactivity probe. In this aspect, the tetrazine-based reactivity probe is selected from

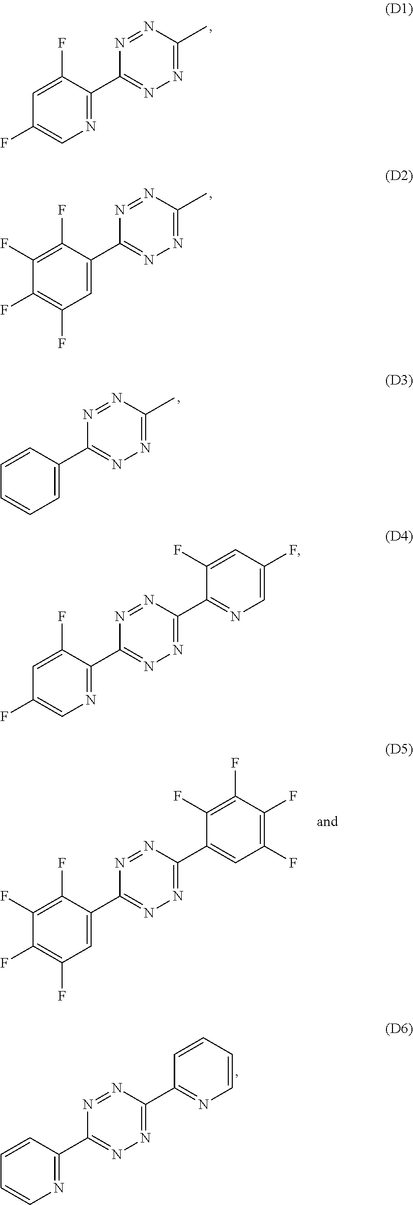 [SMILES: Cc1nnc(-c2ncc(F)cc2F)nn1 ; Cc1nnc(-c2cc(F)c(F)c(F)c2F)nn1 ; Cc1nnc(-c2ccccc2)nn1 ; Fc1cnc(-c2nnc(-c3ncc(F)cc3F)nn2)c(F)c1 ; Fc1cc(-c2nnc(-c3cc(F)c(F)c(F)c3F)nn2)c(F)c(F)c1F ; c1ccc(-c2nnc(-c3ccccn3)nn2)nc1]

or a combination thereof.

In one aspect, the step of determining the structure of the at least one unknown labeled metabolite includes at least one selected from the group consisting of mass spectrometry, UV-VIS spectroscopy, nucleic resonance spectrometry and infrared spectroscopy, or combinations thereof.

In a second aspect, a natural product comprising NP—[X]ₙ identified with the foregoing disclosed method presented herein is provided.

In a third aspect, a composition is disclosed including cyclothiazomycin C having the structure of Formula (I):

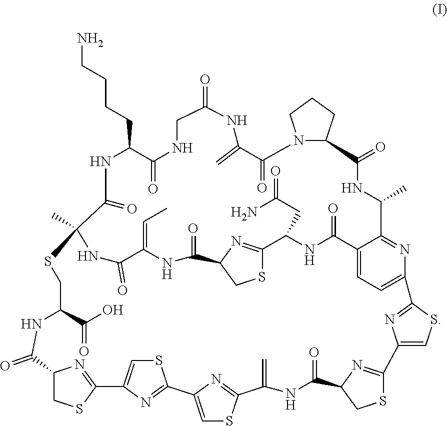 [SMILES: C=C1NC(=O)CNC(=O)[C@H](CCCCN)NC(=O)[C@@]2(C)NC(=O)/C(=C/C)NC(=O)[C@@H]3CSC(=N3)[C@H](CC(N)=O)NC(=O)c3ccc(nc3[C@@H](C)NC(=O)[C@@H]3CCCN3C1=O)-c1nc(cs1)C1=N[C@@H](CS1)C(=O)NC(=C)c1nc(cs1)-c1nc(cs1)C1=N[C@H](CS1)C(=O)N[C@H](C(=O)O)CS2]

In a fourth aspect, a method of identifying a natural product comprising NP—[X]ₙ is disclosed. The method includes several steps. The first step includes preparing a sample suspected to contain NP—[X]ₙ comprising a complex cellular metabolite mixture from an organism. The second step includes reacting the sample suspected to contain NP—[X]ₙ with reactivity probe Y according to Scheme I:

NP—[X]ₙ+Y→NP—[X]ₙ₋ₘ[Z]ₘ Scheme I.

NP—[X] ₙ represents a natural product NP having a chemical moiety X that is susceptible to chemical modification by reactivity probe Y to form at least one product adduct NP—[X]ₙ₋ₘ[Z]ₘ in which chemical moiety X reacts with reactivity probe Y to form adduct Z, wherein n ranges from 1 to about 10 and m is at least 1 and m≦n. The third step, which is optional in some cases, includes dereplicating the product collection of at least one known labeled metabolite to provide a depleted product collection comprising at least one unknown labeled metabolite. The fourth step includes determining the structure of the at least one unknown labeled metabolite, thereby identifying the natural product comprising NP—[X]ₙ.

In one aspect, the step of dereplicating the product collection of at least one known labeled metabolite includes two steps. The first step includes identifying the presence in the product collection including labeled metabolites the at least one known labeled metabolite having a mass of a labeled natural product predicted from a precursor peptide product from the organism selected using the bioinformatics algorithm. The second step includes removing the at least one known labeled metabolite from further characterization.

In a refinement of this aspect, the step of identifying the presence in the product collection including labeled metabolites the at least one known labeled metabolite includes applying differential mass spectrometry to characterize the at least one known labeled metabolite.

In one aspect, the step of dereplicating the product collection of at least one known labeled metabolite includes applying differential mass spectrometry to characterize the product collection.

In one aspect, the organism is selected from bacteria, fungi, plant cells and animal cells. In another aspect, the organism is selected from plant cells, animal cells, and parasite-infected host cells derived plant cells or animal cells.

In one aspect, reactivity probe Y has the structure of Formula (I):

R-L-Q (I),

wherein R is a reactive moiety that reacts with chemical moiety X, L is a linker and Q is a label.

In one refinement of this aspect, the label Q is selected from an affinity label, a detectable group and a physicochemical label. In one aspect, label Q includes an affinity probe. In one refinement of this aspect, the affinity probe is selected from biotin, streptavidin, polyhistine (for example, (His₆)), an unreacted thiol group of dithiothreitol, glutathione-S-transferase (GST), HaloTag®, AviTag, Calmodulin-tag, polyglutamate tag, FLAG-tag, HA-tag, Myc-tag, S-tag, SBP-tag, Softag 3, V5 tag, Xpress tag, and a hapten. In a further refinement of this aspect, the affinity probe includes Formula (A):

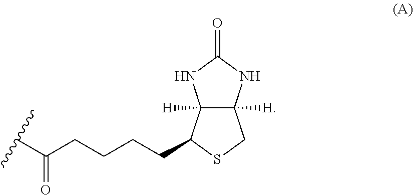

In another refinement, label Q includes a detectable group. In one refinement of this aspect, detectable group is selected from a radiolabel, a fluorescent label, and a chemiluminescent label. In another refinement of this aspect, the detectable group includes a fluorescent label. In yet a further refinement of this aspect, the fluorescent label includes Formula (B):

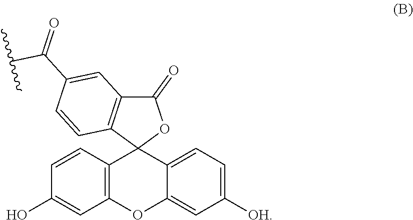 [SMILES: *C(=O)c1ccc2c(c1)C(=O)OC21c2ccc(O)cc2Oc2cc(O)ccc21]

In another refinement, label Q includes a physicochemical label. In one refinement of this aspect, the physicochemical label is selected from an isotopic label and a mass label. In a further refinement of this aspect, the physicochemical label includes a cation mass label. In yet a further refinement of this aspect, the cation mass label includes Formula (C):

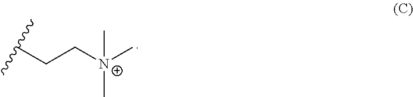 [SMILES: *CC[N+](C)(C)C]

In one aspect, label Q is selected from the following:

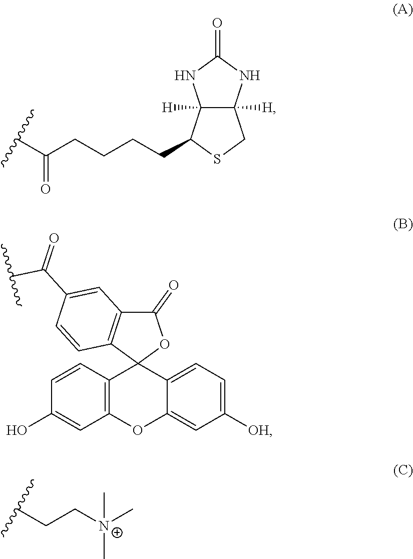

and combinations thereof.

In one aspect, reactivity probe Y is selected from the following:

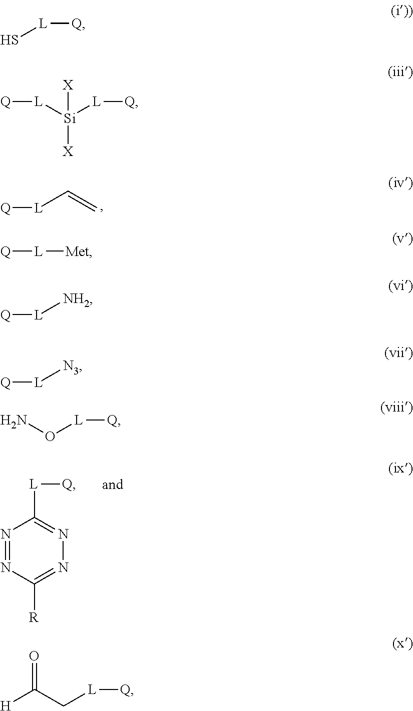

or a combination thereof,
wherein R is alkyl or L-Q.

In one aspect, reactivity probe Y is selected from an aminooxy-based reactivity probe, an aldehyde-based reactivity probe, a thiol-based reactivity probe and a tetrazine-based reactivity probe, or a combination thereof.

In another aspect, reactivity probe Y comprises an aminooxy-based reactivity probe. In this aspect, the aminooxy-based reactivity probe is selected from

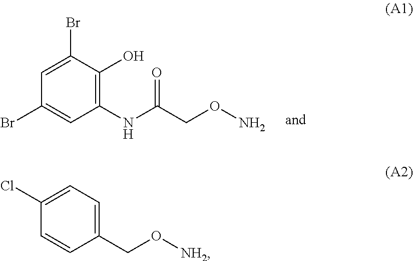 [SMILES: NOCC(=O)Nc1cc(Br)cc(Br)c1O ; NOCc1ccc(Cl)cc1]

or a combination thereof.

In another aspect, reactivity probe Y comprises an aldehyde-based reactivity probe. In this aspect, the aldehyde-based reactivity probe is

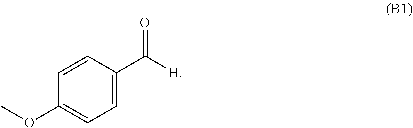

In another aspect, reactivity probe Y comprises a thiol-based reactivity probe. In this aspect, the thiol-based reactivity probe is selected from

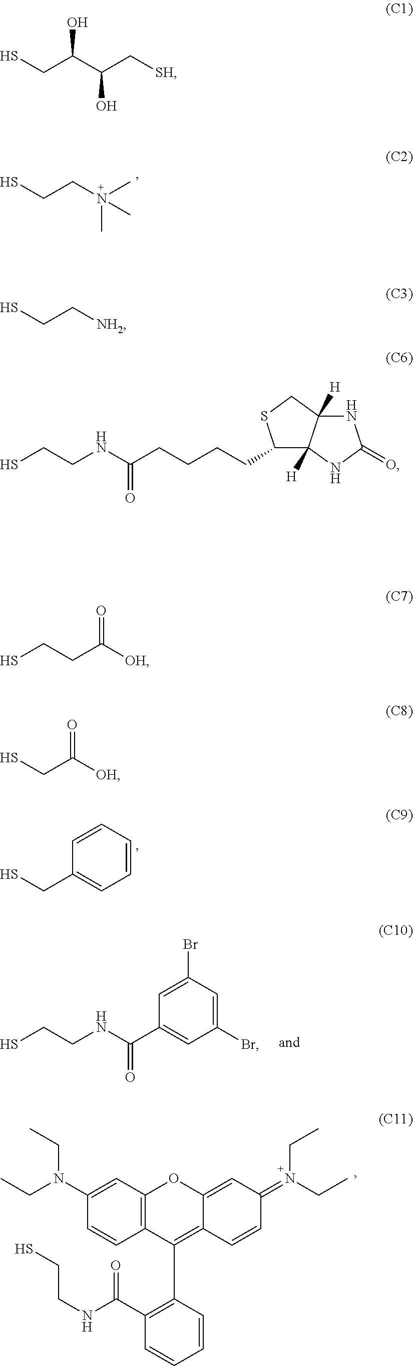

or a combination thereof.

In one aspect, reactivity probe Y comprises a tetrazine-based reactivity probe. In this aspect, the tetrazine-based reactivity probe is selected from

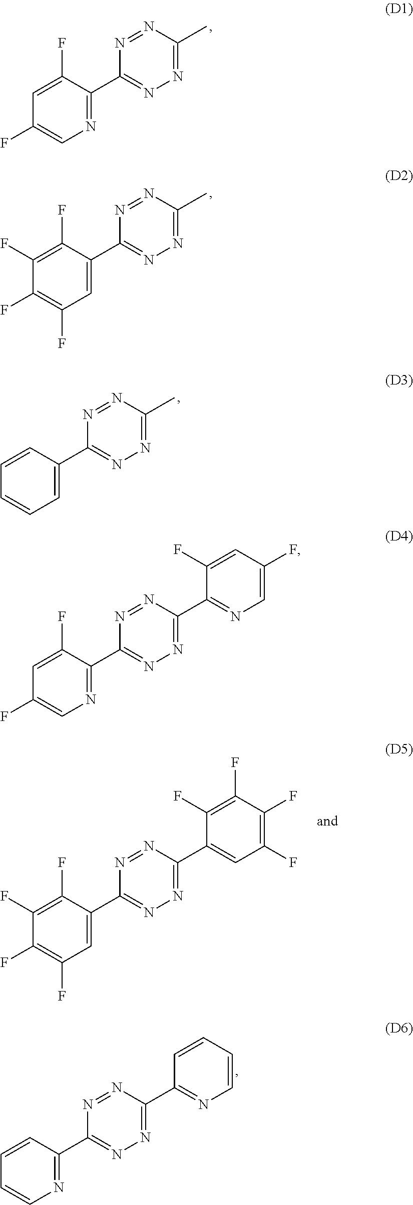 [SMILES: Cc1nnc(-c2ncc(F)cc2F)nn1 ; Cc1nnc(-c2cc(F)c(F)c(F)c2F)nn1 ; Cc1nnc(-c2ccccc2)nn1 ; Fc1cnc(-c2nnc(-c3ncc(F)cc3F)nn2)c(F)c1 ; Fc1cc(-c2nnc(-c3cc(F)c(F)c(F)c3F)nn2)c(F)c(F)c1F ; c1ccc(-c2nnc(-c3ccccn3)nn2)nc1]

or a combination thereof.

In one aspect, the step of determining the structure of the at least one unknown labeled metabolite includes at least one selected from the group consisting of mass spectrometry, UV-VIS spectroscopy, nucleic resonance spectrometry and infrared spectroscopy, or combinations thereof.

In a fifth aspect, a natural product comprising NP—[X]ₙ identified with the foregoing disclosed method presented herein is provided.

### EXAMPLES

### Example 1

### General Methods.

All chemicals were purchased from Sigma-Aldrich, VWR, or Fisher Scientific and used without further purification unless otherwise specified. Compound purification by column chromatography was conducted using either silica or via MPLC (TeleDyne Isco Combi-Flash Rf using normal phase silica or reversed-phase C18-functionalized silica columns). ¹H and ¹³C NMR spectra were collected on Varian Inova 400 MHz or 500 MHz spectrometers. All ¹H and ¹³C spectra were referenced to the solvent peaks. High-resolution mass spectrometry (HRMS) data were obtained on a Micromass Q-TOF Ultima tandem quadrupole mass-spectrometer at the University of Illinois at Urbana-Champaign Mass Spectrometry Laboratory. MALDI-TOF mass spectrometry was performed using a Bruker Daltonics UltrafleXtreme MALDI instrument using Bruker flexControl software for data acquisition and Bruker flexAnalysis software for data analysis. The instrument was calibrated before data acquisition using a commercial peptide calibration kit (AnaSpec—Peptide Mass Standard Kit). Spectra were acquired in positive reflector mode.

### Example 2

Preparation of cell extracts for screening.

Actinomycete strains were grown in 10 mL of MS medium (1 L contains 20 g mannitol, 20 g roasted soy flour) at 30° C. for 7 d. Exported metabolites were extracted from the cultures using 2 mL of n-BuOH at room temperature. For thiostrepton production, Streptomyces azureus was grown in 10 mL of ISP4 medium (1 L contains 10 g soluble starch, 1 g K₂HPO₄, 1 g MgSO₄, 1 g NaCl, 2 g Na₂SO₄, 2 g CaCO₃, 1 mg FeSO₄, 1 mg ZnSO₄ heptahydrate, 1 mg MnCl₂ heptahydrate) for 7 d at 30° C. Thiostrepton was extracted with 1 mL of CHCl₃ at 23° C. Both extracts were agitated for 1 min by vortex, submitted to centrifugation (4000×g, 5 min), and the organic layer was removed from the intact, harvested cells. For geobacillin I production, Geobacillus sp. M10EXG was grown on modified LB agar (1 L contains 10 g casein enzymatic hydrolysate, 5 g yeast extract, 5 g NaCl and 10 g agar) at 50° C. for 60 h. Celts were removed from the plates with 10 ml, of 70% aq, i-PrOH and agitated by rocking for 24 h at 23° C. The intact cells were then removed from the extract by centrifugation (4000×g, 5 min). An aliquot (1 μL) of the extract was then mixed with 9 μL of sat. α-cyano-4-hydroxycinnamic acid (CHCA) matrix solution in 1:1 MeCN/H₂O containing 0.1% trifluoroacetic acid (TFA). 1 μL was spotted onto a MALDI plate for subsequent MALDI-TOF MS analysis.

### DTT-labeling.

For commercially-obtained thiostrepton (Calbiochem, 99%), a 20 μL volume of 10.5 mM thiostrepton, 500 mM DTT, and 10 mM DIPEA in 1:1 CHCl₃/MeOH was allowed to react at 23° C. for 16 h. For the no base reaction, thiostrepton and DTT were added similarly to above and MeOH (without DIPEA) was added to establish a 1:1 CHCl₃/MeOH. The sample was then analyzed for DTT incorporation by MALDI-TOF MS (see below). For thiostrepton produced by Streptomyces azureus (and thus labeling occurred in the context of the crude cell-surface extract), 14 μL of the extract was mixed with DTT (in MeOH) and DIPEA (in MeOH) to generate a final volume of 20 μL with a final concentration of 500 mM DTT and 10 mM DIPEA, in 7:3 CHCl₃/MeOH and the mixture was allowed to proceed for 16 h at 23° C. An aliquot (1 μL) of the extract was then mixed with 9 μL of sat. α-cyano-4-hydroxycinnamic acid (CHCA) matrix solution in 1:1 MeCN/H₂O containing 0.1% TFA. 1 μL was spotted onto a MALDI plate for subsequent MALDI-TOF MS analysis. MALDI-TOF mass spectrometric analysis.

MALDI-TOF mass spectrometry was performed using a Bruker Daltonics UltrafleXtreme MALDI-TOF/TOF instrument operating in positive reflector mode. The instrument was calibrated before data acquisition using a commercial peptide calibration kit (AnaSpec—Peptide Mass Standard Kit). Analysis was carried out with Bruker Daltonics flexAnalysis software. All spectra were processed by smoothing and baseline subtraction.

### Example 3

### Bioinformatics Based Strain Prioritization

A previously reported profile Hidden Markov Model and the program HMMER were used to identify the YcaO cyclodehydratase (Pfam PF02624) (Doroghazi, J. R., Metcalf, W. W. (2013) “Comparative genomics of actinomycetes with a focus on natural product biosynthetic genes,” BMC Genomics. 14, 611; Punta, M. et al. (2012) “The Pfam protein families database,” Nucleic Acids Res. 40, D290-301; Eddy, S. R. (1998) Profile hidden Markov models, Bioinformatics. 14, 755-763). The local genomic region (10 open reading frames on either side of the YcaO gene) was analyzed manually for the presence of a “lantibiotic dehydratase” gene and a putative precursor peptide. Only strains with the presence of all three genes were taken forward for reactivity-based screening.

### Example 4

Isolation and characterization of cyclothiazomycin C and cyclothiazomycin B.
Isolation of cyclothiazomycin C.

WC-3908 was grown in 10 mL of ATCC 172 medium at 30° C. for 48 h. 300 μL of the culture was spread onto 15 cm plates (ca. 75 mL of solid ATCC medium). The plates were then incubated for 7 d at 23° C. A razor blade was used to remove the bacterial lawn from the solid medium. The bacterial growth from 14 plates (˜1 L of medium) was extracted with n-BuOH (500 mL) for 24 h at 23° C. The extract was then filtered through Whatman filter paper and allowed to evaporate under nitrogen before being redissolved in 3:1 pyridine:water (ca. 3 mL) and transferred to a 50 mL conical tube. The resulting solution was clarified by centrifugation, to remove insoluble debris (4000×g, 5 min). The supernatant was then injected onto a reverse-phase C18 silica column (TeleDyne Isco 5.5 g C18 Gold cartridge) and purified by MPLC (gradient elution from 20-95% MeOH/10 mM aq. NH₄HCO₃). Fractions containing the desired product (as determined by MALDI-TOF MS; [M+H] m/z=1486) were combined and immediately concentrated by rotary evaporation. The resulting residue was dissolved in 3:1 pyridine/water (ca. 0.5 mL), transferred to a microcentrifuge tube, centrifuged (15000×g, 5 min), filtered (0.2 μm polyethersulfone syringe filter), and further purified by HPLC. Semi-preparative HPLC employed a Thermo Scientific Betasil C18 column (100 Å; 250×10 mm; 5 μm particle size) operating at 4.0 mL min⁻¹ on a PerkinElmer Flexar LC system using Flexar Manager software. Solvent A was 10 mM aq. NH₄HCO₃. Solvent B was MeOH. Cyclothiazomycin C was purified by isocratic elution at 72% B, typically eluting 19.5 min after initiation of the HPLC run (alternatively, the elution time was ˜12 min when 75% B was used). HPLC progress was monitored by photodiode array (PDA) UV-Vis detection. Fractions corresponding to the desired product (as determined by UV-Vis and MALDI-TOF MS) were immediately concentrated under rotary evaporation or under a stream of N₂ gas. The resulting residue was suspended in water (ca. 1 mL), assisted by vortex mixing and sonication. The suspended product was flash-frozen in liquid N₂ and lyophilized for >24 h to give purified cyclothiazomycin C as a white to off-white powder. Purity was determined by analytical HPLC [Thermo Scientific Betasil C18 column (100 Å; 250×4.6 mm; 5 μm particle size) operating at 1.0 mL min⁻¹ using the same solvents] and NMR. Isolated yield ranged from 10-90 μg/plate (15 cm diameter).

Isolation of cyclothiazomycin B.

NRRL strain B-3306 was grown in a fashion identical isolation conditions for WC-3908. Cyclothiazomycin B ([M+H] m/z=1528) was also purified in the same manner as cyclothiazomycin C, except that HPLC purification employed 75% B (retention time typically ca. 17 min). After lyophilization, an off-white powder was obtained. Purity was determined by analytical HPLC [Thermo Scientific Betasil C18 column (100 Å; 250×4.6 mm; 5 μm particle size) operating at 1.0 mL min⁻¹ using the same solvents]; identity was determined by high-resolution mass spectrometry. Isolated yield was approximately 13 μg/plate (15 cm diameter).

FT-MS/MS analysis of cyclothiazomycin B and C.

The purified cyclothiazomycins were dissolved in 80% aq. MeCN with 0.1% formic acid. Samples were directly infused using a 25 μL Hamilton gas-tight syringe (cyclothiazomycin C) or an Advion Nanomate 100 (cyclothiazomycin B), into a ThermoFisher Scientific LTQ-FT hybrid linear ion trap, operating at 11T (calibrated weekly). The FT-MS was operated using the following parameters: minimum target signal counts, 5,000; resolution, 100,000; m/z range detected, dependent on target m/z; isolation width (MS/MS), 5 m/z; normalized collision energy (MS/MS), 35; activation q value (MS/MS), 0.4; activation time (MS/MS), 30 ms. Data analysis was conducted using the Qualbrowser application of Xcalibur software (Thermo-Fisher Scientific).

NMR spectroscopy of cyclothiazomycin C.

NMR spectra were recorded on a Varian NMR System 750 MHz narrow bore magnet spectrometer (VNS750NB employing a 5 mm Varian 1H[13C/15N] PFG X, Y, Z probe) or a Varian Unity Inova 500 MHz narrow bore magnet spectrometer (UI500NB employing a 5 mm Varian 1H[13C/15N] PFG Z probe). Spectrometers were operated at 750 MHz and 500 MHz, respectively, for ¹H detection, and 188 MHz for indirect ¹³C detection. Carbon resonances were assigned via indirect detection (HSQC and HMBC experiments). Resonances were referenced internally to the most downfield solvent peak (8.74 ppm, pyridine). Default Varian pulse sequences were employed for ¹H, COSY, DQF-COSY, TOCSY, HSQC, HMBC, and ROESY experiments. Samples were prepared by dissolving approximately 3-7 mg of cyclothiazomycin C (HPLC-purified and lyophilized) in pyridine-d5/D2O (3:1, 600 μL). Pyridine-d5 (99.94% D) and D2O (99.9% D) were obtained from Cambridge Isotope Laboratories (Andover, Mass.). Samples were held at 25° C. during acquisition.

Analysis of NMR data.

Assigned resonances are shown in tabular form and directly on the structure within FIG. 13. Due to the solvent employed (3:1 pyridine-d5/D2O), exchangeable peaks (i.e. N—H, O—H) were not detected. The corresponding ¹H resonances of the analogous locations in cyclothiazomycin B1 (reported previously (1)) are also given in FIG. 13 for comparison. Resonances were assigned by 2D NMR spectroscopy, as well as by comparison to the reported spectra of cyclothiazomycin B1 (1).

Evaluation of cyclothiazomycin B and C antibiotic activity.

Bacillus subtilis strain 168, Bacillus anthracis strain Sterne, E. coli MC4100, and Pseudomonas putida KT2440 were grown to stationary phase in 10 mL of Luria-Bertani broth (LB) at 37° C. Staphylococcus aureus USA300 (methicillin-resistant), Enterococcus faecalis U503 (vancomycin-resistant), and Listeria monocytogenes strain 4b F2365 were grown to stationary phase in 10 mL brain-heart infusion (BHI) medium at 37° C. Neisseria sicca ATCC 29256 was grown to stationary phase in 5 mL of gonococcal broth at 37° C. The cultures were adjusted to an OD600 of 0.013 in the designated medium before being added to 96-well microplates. Successive two-fold dilutions of cyclothiazomycin C or cyclothiazomycin B (standard solution: 5 mg m L⁻¹ in DMSO) were added to the cultures (0.5-64 μg mL⁻¹). As a control, kanamycin was added to samples of E. coli, B. subtilis, B. anthracis, P. putida, L. monocytogenes, and N. sicca with dilutions from 1-32 μg mL⁻¹. Gentamycin was used as a control for S. aureus and E. faecalis. As a negative control, an equal volume of DMSO lacking antibiotic was used. Plates were covered and incubated at 37° C. for 12 h with shaking. The minimum inhibitory concentration (MIC) reported is the value that suppressed all visible growth.

Evaluation of cyclothiazomycin B and C antifungal activity.

Saccharomyces cerevisiae, Talaromyces stipitatus, and Aspergillus niger were grown for 36 h in 2 mL of YPD medium (1 L contains 10 g yeast extract, 20 g Peptone and 20 g Dextrose) at 30° C. Fusarium virguliforme was grown for 7 d on potato dextrose agar at 30° C. Spores were isolated and a suspension of 10⁶ spores in potato dextrose broth was added to the 96-well microplate. S. cerevisiae cultures were adjusted to an OD600 of 0.013 in the designated medium before being added to 96-well microplates. T. stipitatus, and A. niger were not diluted prior to adding to the 96-well microplate. Successive two-fold dilutions of cyclothiazomycin C and cyclothiazomycin B (standard solution: 5 mg mL⁻¹ in DMSO) were added to the cultures (0.5-64 μg mL⁻¹). As a positive control, amphotericin B was added to the cultures with dilutions from 0.5-8 μg mL⁻¹. An equal volume of DMSO was used as a negative control. Plates were covered and incubated at 30° C. for 36 h for T. stipitatus, A. niger, and S. cerevisiae or 60 h for F. virguliforme with shaking. The minimum inhibitory concentration (MIC) reported is the value that suppressed all visible growth.

### Example 5

Aminooxy-based reactivity probe designs, syntheses and applications.

### Example 5.1

### Aminooxy-Based Reactivity Probes Synthesis

Compounds were prepared as described below, except for 1-[(aminooxy)methyl]-4-chlorobenzene hydrochloride (A3), which was obtained from a commercial vendor (e.g., Santa Cruz Biotechnology [US]).

### Tert-butyl (2-((3,5-dibromo-2-hydroxyphenyl)amino)-2-oxoethoxy)carbamate (Al)

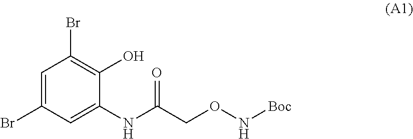 [SMILES: CC(C)(C)OC(=O)NOCC(=O)Nc1cc(Br)cc(Br)c1O]

To a solution of (boc-aminooxy)acetic acid (200 mg, 1.05 mmol) in dry tetrahydrofuran (10 mL) was added 2-amino-4,6-dibromophenol (294 mg, 1.10 mmol), 1-ethyl-3-(3-dimethylaminopropyl)carbodiimide hydrochloride (220 mg, 1.15 mmol), and hydroxybenzo-triazole hydrate (186 mg, 1.15 mmol). The solution was stirred at room temperature for 18 h. The reaction was then taken up in ethyl acetate and washed twice with saturated sodium bicarbonate and once with brine. The ethyl acetate fraction was dried over sodium sulfate and concentrated by rotary evaporation. The product was purified by silica flash column chromatography (gradient of 0-25% ethyl acetate in hexanes) to yield Al as an orange solid (292 mg, 63%). ¹H NMR (500 MHz, CDCl₃) δ ppm 10.66 (br, 1H), 9.64 (br, 1H), 7.92 (s, 1H), 7.52 (d, J=2.5 Hz, 1H), 7.43 (d, J=2 Hz, 1H), 4.52 (s, 2H), 1.52 (s, 9H). ¹³C NMR (500 MHz, CDCl₃) δ ppm 169.49, 158.67, 145.26, 132.43, 127.16, 124.61, 114.28, 111.52, 84.51, 76.28, 28.08. HRMS (m/z): [M+Na]⁺ calc. for C₁₃H₁₆N₂O₅Br₂Na, 460.9324; observed, 460.9320.

### 2-(aminooxy)-N-(3,5-dibromo-2-hydroxyphenyl)acetamide (A2)

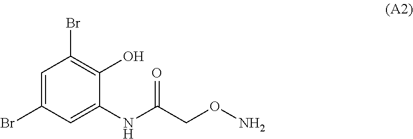 [SMILES: NOCC(=O)Nc1cc(Br)cc(Br)c1O]

The Boc-protected probe A1 (122 mg, 0.277 mmol) was dissolved in 4 M HCl in dioxane (3 mL) and stirred at room temperature for 3 h. The reaction was taken up in ethyl acetate and washed twice with saturated sodium bicarbonate and once with brine. The ethyl acetate fraction was dried over sodium sulfate and concentrated by rotary evaporation. The product was purified by silica flash column chromatography (gradient of 0-5% methanol in dichloromethane) to yield 2 as a white solid (52 mg, 55%). ¹H NMR (500 MHz, (CD₃)₂SO) δ ppm 8.02 (d, J=2 Hz, 1H), 7.51 (d, J=2.5 Hz, 1H), 4.18 (s, 2H). ¹³C NMR (500 MHz, (CD₃)₂SO) δ ppm 169.83, 144.49, 129.60, 129.35, 123.62, 112.33, 110.91, 74.31. HRMS (m/z): [M−H]⁻ calc. for C₈H₇N₂O₃Br₂, 336.8823; observed, 336.8816.

### Example 5.2

### Labeling of Carbonyl-Containing Compounds Via Aminooxy-Based Reactivity Probes

The reaction scheme for labeling of carbonyl compounds (aldehydes and ketones) with aminooxy-based reactivity probes is presented in FIG. 2. Compounds containing ketone or aldehyde carbonyl moieties can be covalently labeled by aminooxy probes, forming O-substituted oximes. The usefulness of the aminooxy probes was first demonstrated by labeling of representative carbonyl-bearing natural products. Labeling reactions for streptomycin, daunomycin, and 4-anisaldehyde were prepared in water, MeOH, or EtOH with a final concentration of 1 μM aldehyde or ketone and 1 mM probe A2 from 10× stocks (in water or EtOH). The choice of reaction solvent did not significantly affect labeling. The reactions were run at rt for 3 h with occasional manual shaking before being analyzed by MALDI-TOF MS. Labeling was verified by the presence of mass shifts in the reacted material relative to the unreacted material consistent with the addition of the probe and loss of water and by the presence of peaks containing isotope distributions corresponding to the presence of two bromine atoms.

Representative carbonyl-bearing natural products labeled via reaction with aminooxy-based reactivity probes are illustrated below.

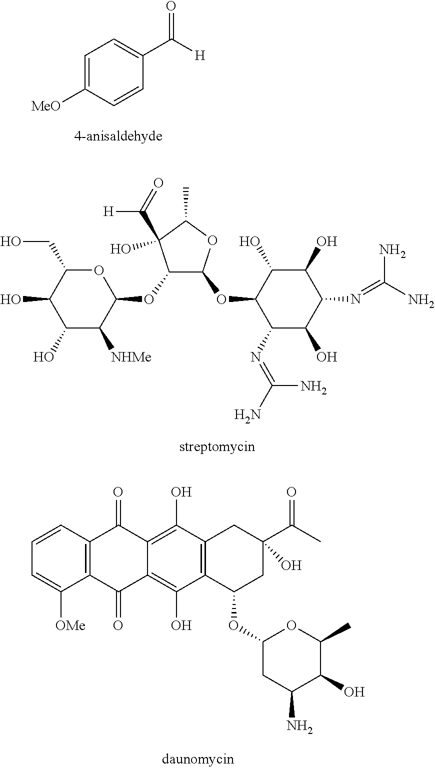 [SMILES: COc1ccc(C=O)cc1 ; CN[C@@H]1[C@H](O[C@H]2[C@H](O[C@H]3[C@H](O)[C@@H](O)[C@H](N=C(N)N)[C@@H](O)[C@@H]3N=C(N)N)O[C@@H](C)[C@]2(O)C=O)O[C@@H](CO)[C@H](O)[C@H]1O ; COc1cccc2c1C(=O)c1c(O)c3c(c(O)c1C2=O)C[C@@](O)(C(C)=O)C[C@@H]3O[C@H]1C[C@H](N)[C@H](O)[C@H](C)O1]

Representative labeling of streptomycin with a brominated aminooxy-based reactivity probe is illustrated below in Scheme II.

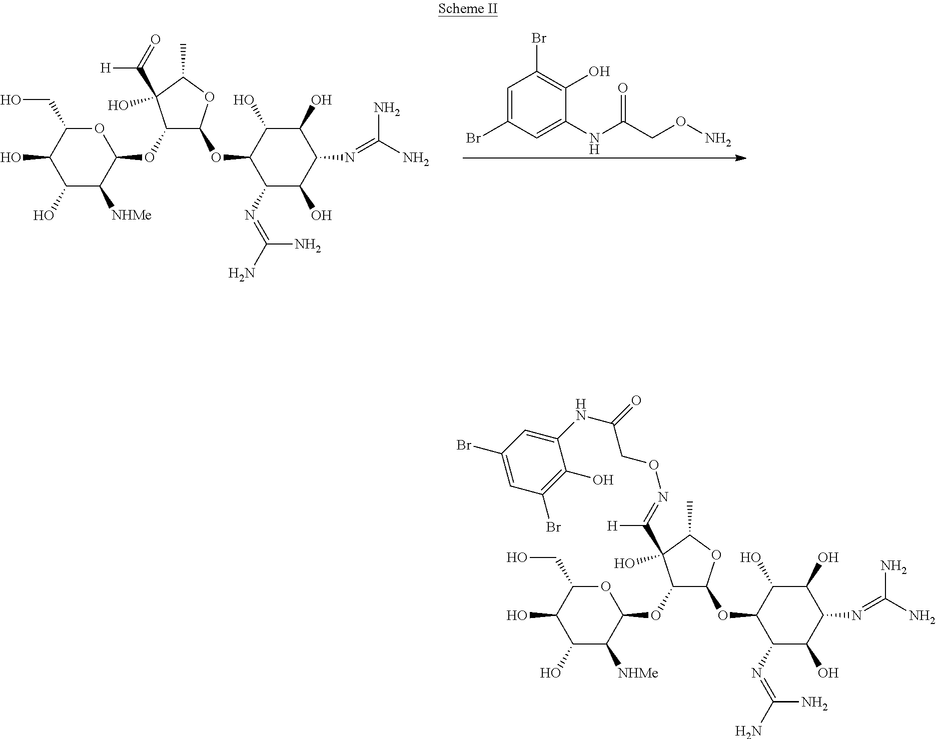 [SMILES: CN[C@@H]1[C@H](O[C@H]2[C@H](O[C@H]3[C@H](O)[C@@H](O)[C@H](N=C(N)N)[C@@H](O)[C@@H]3N=C(N)N)O[C@@H](C)[C@]2(O)C=O)O[C@@H](CO)[C@H](O)[C@H]1O ; NOCC(=O)Nc1cc(Br)cc(Br)c1O ; CN[C@@H]1[C@H](O[C@H]2[C@H](O[C@H]3[C@H](O)[C@@H](O)[C@H](N=C(N)N)[C@@H](O)[C@@H]3N=C(N)N)O[C@@H](C)[C@]2(O)/C=N/OCC(=O)Nc2cc(Br)cc(Br)c2O)O[C@@H](CO)[C@H](O)[C@H]1O]

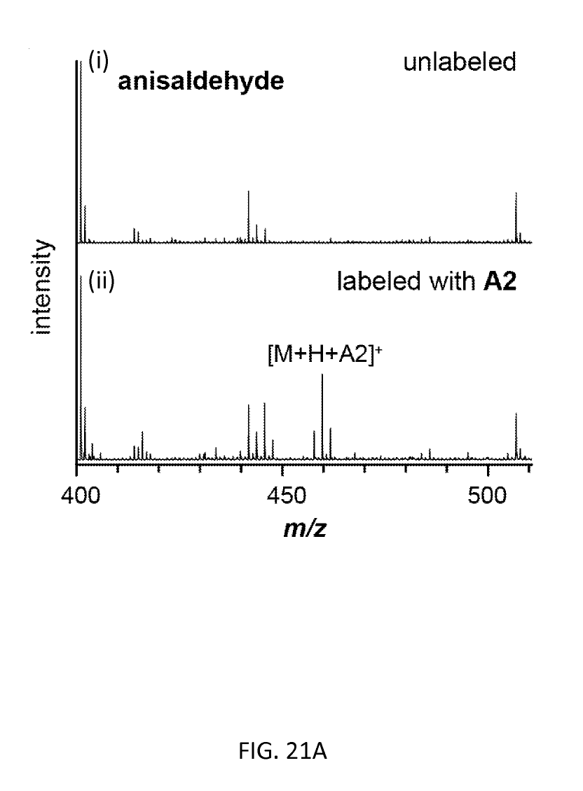

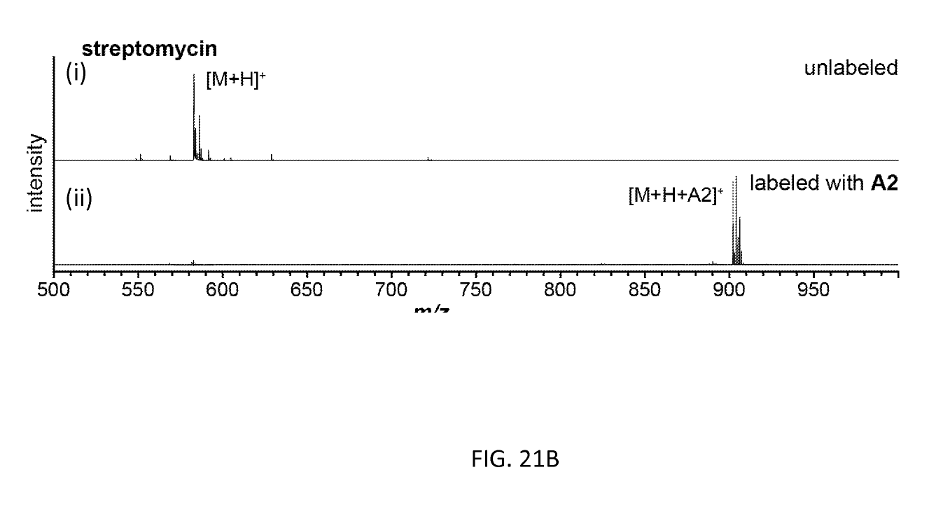

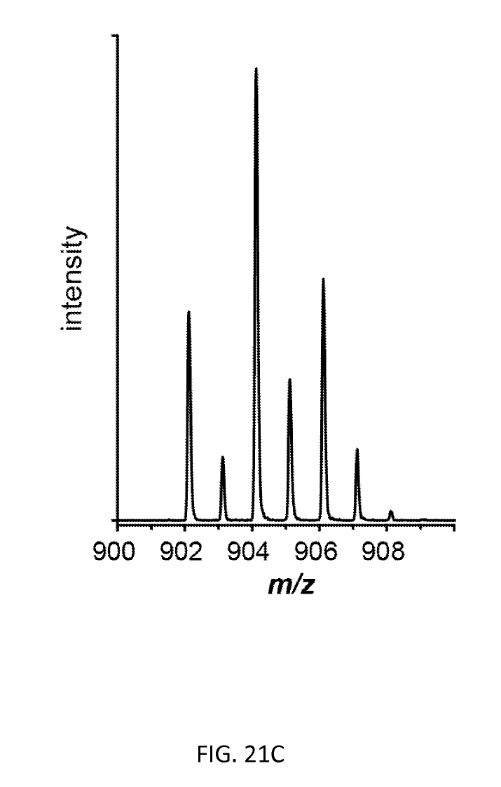

Representative labeling of aldehyde-bearing natural products with aminooxy-based reactivity probes is shown in FIG. 21.

### Example 5.3

Screening of bacterial extracts for carbonyl-containing compounds via aminooxy-based reactivity probes.

A previously described collection of ˜400 extracts of actinobacteria¹ (not prioritized by bioinformatics) was screened using the aminooxy probe A2 for the presence of carbonyl-bearing natural products. The extracts had been partially purified on Oasis HLB extraction columns (Waters) and were dissolved in 50% aq. MeCN. Labeling reactions were set up with 9 μL of extract solution and 1 μL of A2 from a 10 mM stock in EtOH in 0.2 mL tubes. The reactions were run for at least 3 h at rt with occasional manual shaking. Each reaction was analyzed by MALDI-TOF MS. Spectra were analyzed for peaks displaying an isotope pattern consistent with the presence of two bromine atoms.

Initial hits in the screen were verified by a follow up screen using the same conditions as above but with the commercially available probe

1-[(aminooxy)methyl]-4-chlorobenzene hydrochloride (A3).

Upon successful labeling with both probes A2 and A3, the producing organisms were grown in scaled-up cultures for the production of the compounds on a larger scale. Seed cultures of these actinobacteria (5 mL) were grown in ATCC media no. 172 (10 g/L glucose, 20 g/L soluble starch, 5 g/L yeast extract, 5 g/L N-Z amine type A [Sigma C0626], 1 g/L CaCO₃, pH 7.3) at 30° C. on a tube roller for 4-7 d. A 1 mL portion of the seed cultures were used to inoculate 15 cm diameter agar plates (60 mL media per plate) of ATCC media no. 172 (with 15 g/L agar), ISP media no. 4 (10 g/L soluble starch, 1 g/L K₂HPO₄, 1 g/L MgSO₄.7H₂O, 1 g/L NaCl, 2 g/L (NH₄)₂SO₄, 2 g/L CaCO₃, 1 mg/L FeSO₄.7H₂O, 1 mg/L ZnSO₄.7H₂O, 1 mg/L MnCl₂.4H₂O, 15 g/L agar, pH 7.2), or MS (10 g/L mannitol, 10 g/L soy flour [Kinako, Wel-Pac], 10 g/L malt extract, 15 g/L agar). Agar plates were grown at 30° C. for 10 d. Bacteria and the top layer of agar were scraped off the plate and extracted with MeOH overnight. Solid material was removed by centrifugation at 20,000×g for 30 min followed by careful removal of the liquid extract and concentration under reduced pressure.

For purification, the clarified extracts were adsorbed onto Celite 545 and purified by reversed-phase MPLC (50 g C18 Gold media; Teledyne Isco) with a CombiFlash Rf 200 (Teledyne Isco). Chromatography was performed with a flow rate of 40 mL/min using a gradient of 10-100% aq. MeOH. Fractions containing the desired natural product, as determined by MALDI-TOF MS, were pooled and concentrated. The solid was dissolved in water and loaded onto a reversed-phase HPLC column (Betasil C18, 10 mm×250 mm, 100 Å pore size, 5 μM particle size; Thermo Scientific). Chromatography was performed with a flow rate of 4 mL/min using H₂O with 0.1% formic acid (solvent A) and MeOH with 0.1% formic acid (solvent B) with a gradient of: time 0 min, 5% B; time 5 min, 5% B; time 45 min, 95% B; time 50 min, 95% B. Fractions containing the desired natural product were pooled and concentrated.

### Example 6

Aldehyde-based reactivity probe designs, syntheses and applications.

### Example 6.1

### Aldehyde-Based Reactivity Reactivity Probes

Compound 4-anisaldehyde (B1) was obtained from a commercial vendor (e.g., Sigma-Aldrich Co. LLC [US]).

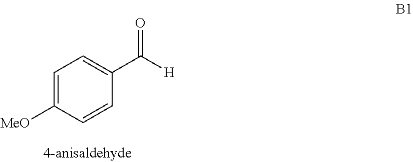

### Example 6.2

Labeling of aminoalcohol-containing compounds via aldehyde-based reactivity probes.

The reaction scheme for labeling of aminoalcohol-containing compounds with aldehyde probes is shown in FIG. 2. In the presence of an aldehyde, 1,2-aminoalcohols can form oxazolidine moieties. The usefulness of the aldehyde probes was first demonstrated by labeling of representative carbonyl-bearing natural products.

Labeling reactions for doxorubicin, kanamycin, and vancomycin were prepared in H₂O, MeOH, or EtOH with a final concentration of 1 mM natural product and 100 mM probe (anisaldehyde, B1) from 10× stocks (in MeOH). The reactions were run at rt or elevated temperature (60° C.) for ca. 1 h without stirring. Reaction progress was analyzed by MALDI-TOF MS. Representative 1,2-aminoalcohol-bearing natural products labeled via reaction with aldehyde probes are depicted below.

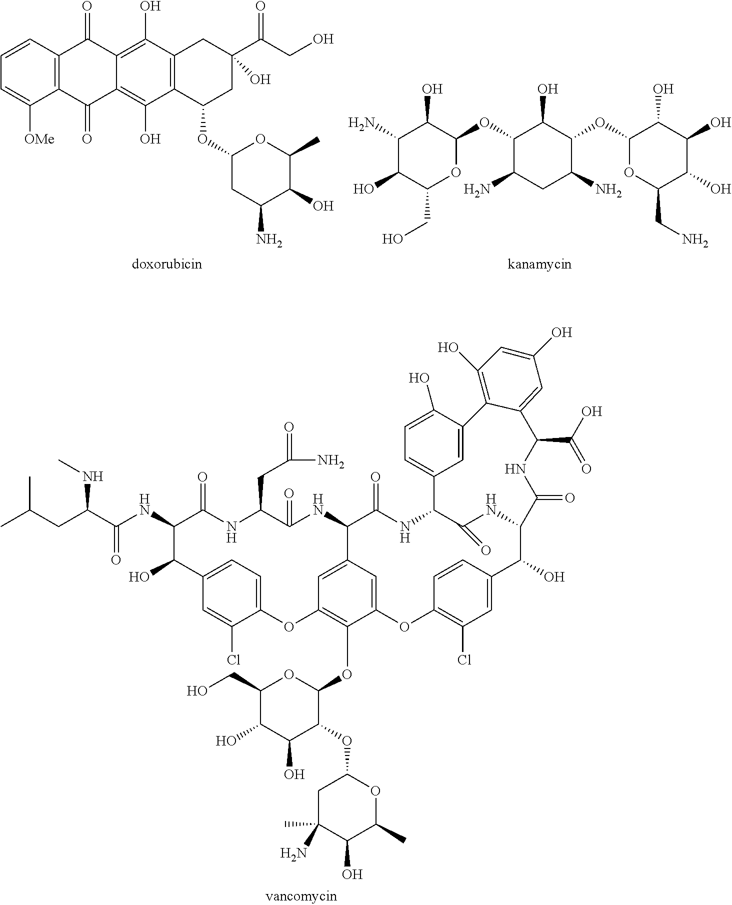 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1 ; NC[C@H]1O[C@H](O[C@H]2[C@H](O)[C@@H](O[C@H]3O[C@H](CO)[C@@H](O)[C@H](N)[C@H]3O)[C@H](N)C[C@@H]2N)[C@H](O)[C@@H](O)[C@@H]1O ; CN[C@H](CC(C)C)C(=O)N[C@H]1C(=O)N[C@@H](CC(N)=O)C(=O)N[C@H]2C(=O)N[C@H]3C(=O)N[C@H](C(=O)N[C@H](C(=O)O)c4cc(O)cc(O)c4-c4cc3ccc4O)[C@H](O)c3ccc(c(Cl)c3)Oc3cc2cc(c3O[C@@H]2O[C@H](CO)[C@@H](O)[C@H](O)[C@H]2O[C@H]2C[C@](C)(N)[C@H](O)[C@H](C)O2)Oc2ccc(cc2Cl)[C@H]1O]

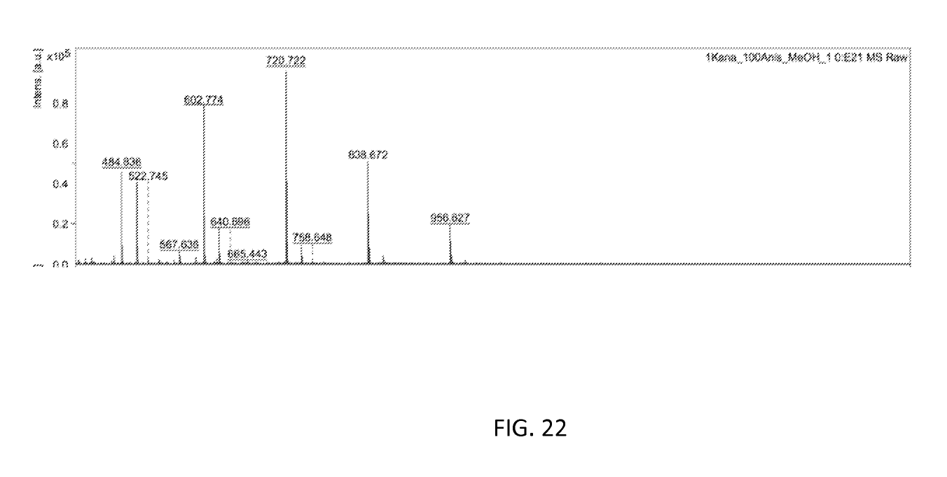

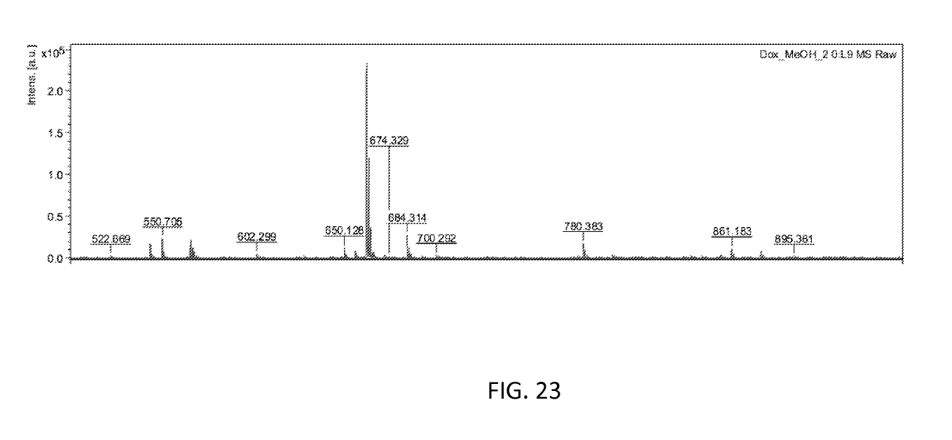

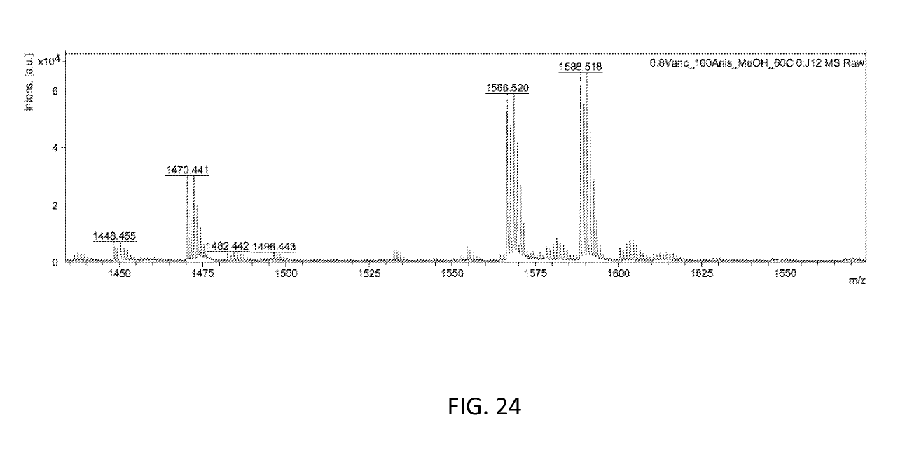

Kanamycin ([M+H]⁺ m/z=485) labeling was evidenced by the appearance of peaks at 603 Da. Additional labels corresponding to imine formation at the other 3 amines in the substrate were seen at 721, 839, and 957 m/z (FIG. 22) Doxorubicin ([M+H]⁺ m/z=544) labeling is evidenced by the appearance of a peak at 662 m/z, consistent with the addition of one anisaldehyde label (FIG. 23). Vancomycin ([M+H]⁺ m/z=1448) labeling is evidenced by the appearance of a peak at 1566 m/z, consistent with the addition of one anisaldehyde label (FIG. 24).

### Example 6.3

Screening of bacterial extracts for aminoalcohol-containing compounds via aldehyde-based reactivity probes.

Labeling with aldehyde probes was also demonstrated in the context of a complex bacterial extract. The amphotericin-producing bacterium Streptomyces nodosus was grown on altMS agar plates at 30° C. for at least 3 d. A whole cell mass spectrum was taken after colony growth. 2 μL matrix (sat. CHCA in 50% aq. MeCN with 0.1% formic acid) was spotted onto a steel plate; then using a sterile wooden stick, a colony was taken from the agar plate and placed onto the spot containing the matrix. Another 2 μL matrix was spotted on top of the colony. The sample was then analyzed via MALDI-TOF mass spectrometry. This served as an unlabeled control. For labeling, 5 μL anisaldehyde in MeOH (probe B1; final concentration 100 mM) and 40 μL MeOH was added to a microfuge tube. 2-3 colonies from the altMS plate were selected and added the tube. The reaction was left for 1 h before an aliquot (1 μL) was admixed with 1 μL matrix, spotted, and analyzed via MALDI-TOF MS. A representative aminoalcohol-containing compound labeled by an aldehyde probe in the context of a bacterial extract is shown below.

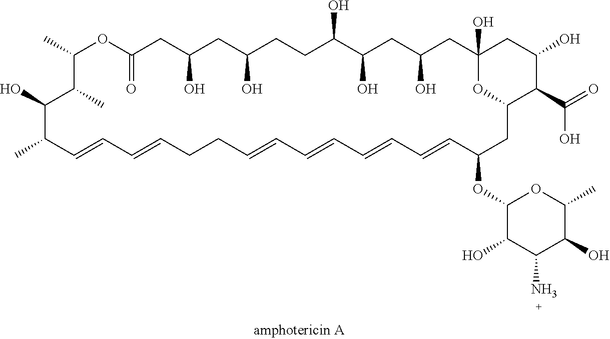

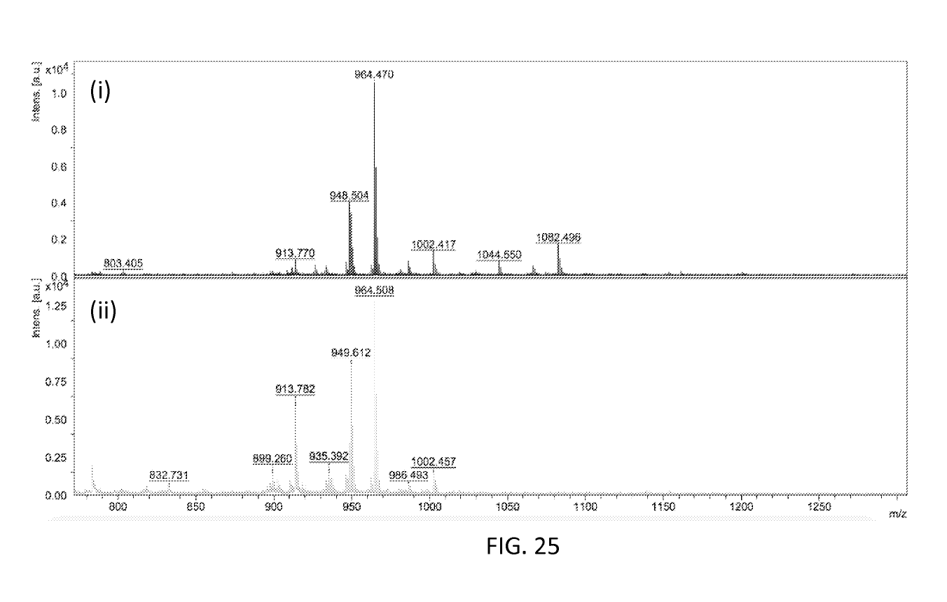

In a mass spectrum of the unlabeled extract, amphotericin A primarily appears as the potassiated adduct [M+K]⁺ m/z=964. In the labeled material, incorporation of a single anisaldehyde (B1) moiety is evidenced by the appearance of a peak at 1082 m/z (FIG. 25).

### Example 7

Thiol-based reactivity probe designs, syntheses and applications.

### Example 7.1

### Thiol-Based Reactivity Probe Syntheses

Compounds were prepared as described below, except for dithiothreitol (DTT) (C1), cysteamine (C3), mercaptopropionic acid (C7) and thioglycolic acid (C8) which were obtained from a commercial vendor (e.g., Sigma-Aldrich Co. LLC [US]).

Thiocholine chloride (C2).² The synthesis of compound (C2) was performed according to Scheme III.

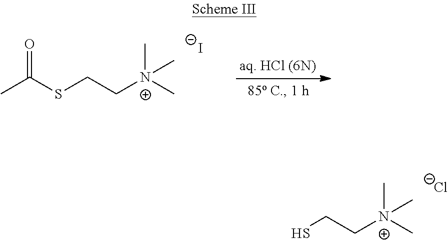

Acetylthiocholine iodide (50 mg) was heated and stirred at 85° C. in 6 N HCl (400 μL) for 1 h. The solvent was removed under a stream of nitrogen. To the resulting solid was added MeOH (ca. 5 mL), which was evaporated to encourage removal of HCl. This was repeated 2 more times before the material was allowed to dry under an N₂ stream overnight. A sample of the resultant white solid was analyzed by ¹H NMR (D₂O, 500 MHz), showing the presence of the desired material in high purity with no detectable amount of the acetylated starting compound. Spectral properties were consistent with the literature.²

Biotin-containing reactivity probe (C6) preparation. The synthesis of the biotin-containing reactivity probe was prepared according to Scheme IV.

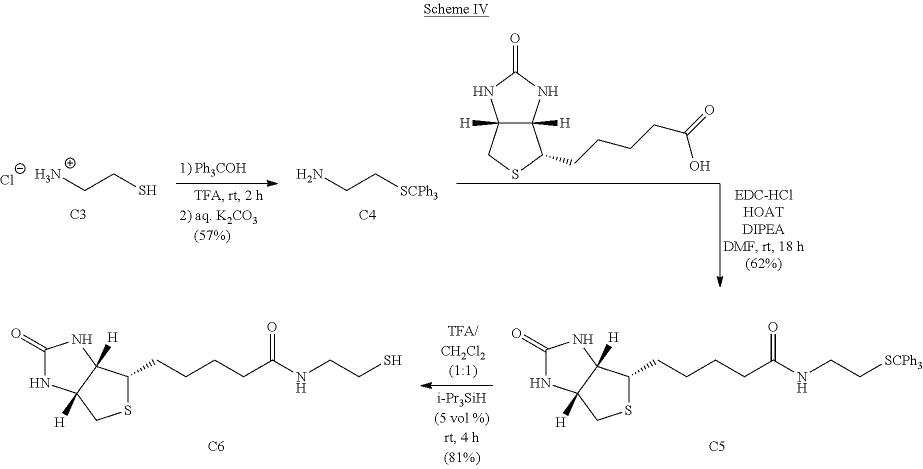

Trityl cysteamine (C4).³ To a scintillation vial under ambient atmosphere was added cysteamine hydrochloride (C3) (363 mg, 3.20 mmol), a stir bar, and trifluoroacetic acid (2 mL). The mixture was stirred and triphenylmethanol (814 mg, 3.13 mmol) was added in portions (addition resulted in a deep sanguine reddening of the reaction mixture). The reaction mixture was allowed to stir at room temperature for 2 h before most of the solvent was evaporated under a stream of nitrogen. The resulting thick, gummy liquid was added to water (30 mL) and solid K₂CO₃ was added until the residual acid was neutralized (pH paper). The resulting solid/liquid mixture was extracted with CH₂Cl₂ (3×20 mL; addition of CH₂Cl₂ resulted in dissolution of all solids), dried over MgSO₄, filtered, and evaporated under reduced pressure to give a pale yellow solid. The material was redissolved in CH₂Cl₂ (ca. 5 mL) and purified by MPLC (4 g silica cartridge; 0-10% MeOH in CH₂Cl₂). The fractions that were estimated to have the desired material were combined and evaporated overnight to yield the pure material as an off-white solid (571 mg, 57%). Spectral data were consistent with the literature.³

Trityl biotin thiol (C5). To a vial equipped with stir bar were added biotin (28.5 mg, 0.117 mmol), EDC hydrochloride (26.2 mg, 0.137 mmol), DIPEA (20.3 uL, 0.117 mmol), trityl cysteamine (38.0 mg, 0.119 mmol), HOAt (16.0 mg, 0.118 mmol), and DMF (1 mL). The resulting yellow solution was stirred at room temperature overnight under ambient atmosphere. The next day (ca. 18 h), the material was partitioned between EtOAc (30 mL) and water (10 mL). The layers were separated and the organic fraction was further washed with brine (2×10 mL). The organic layer was then dried (MgSO₄), filtered, and concentrated. The material was purified by MPLC (0-20% MeOH/CH₂Cl₂) to afford the product as a near-colorless oil which became a white solid (39.3 mg, 62%) upon evaporation from CDCl₃. HRMS (ESI) [M+H]⁺ m/z calcd. 546.2249, found 546.2251 (0.4 ppm) for C₃₁H₃₆N₃O₂S₂.

Biotin thiol (C6).⁴ Trityl biotin thiol (35.4 mg) was dissolved in 1:1 TFA/CH₂Cl₂ (3 mL) with triisopropylsilane (150 μL). The resulting solution was then stirred at room temperature. After 4 h, the material was evaporated under N₂, redissolved in toluene (3 mL), evaporated again, redissolved in CH₂Cl₂ (3 mL), and evaporated overnight. The material was purified by MPLC (4 g silica, 0-20% MeOH/CH₂Cl₂) and the fractions containing a KMnO₄-staining spot were combined and evaporated under reduced pressure. NMR confirmed the presence of the desired material. Drying under reduced pressure afforded the pure product as a white solid (15.87 mg, 81%). Spectral data were consistent with the literature.⁴

### Example 7.2

Labeling of activated alkene-containing compounds via thiol-based reactivity probes.

The reaction scheme for labeling of electron-poor alkene-containing compounds with thiol-based reactivity probes are illustrated in FIG. 2. Dehydrated amino acids (DHAAs), or any similar moieties consisting of alkenes activated by conjugated electron-withdrawing groups, may be labeled by nucleophilic 1,4-addition of a thiol probe in the presence of a mild base. The usefulness of the thiol probes was first demonstrated by labeling of a representative dehydrated amino acid-containing natural product, thiostrepton.

For commercially-obtained thiostrepton (99% pure; Calbiochem, Inc. [US]), a 20 μL volume of 10.5 mM thiostrepton, 500 mM DTT (probe C1), and 10 mM DIPEA in 1:1 CHCl₃/MeOH was allowed to react at 23° C. for 16 h. For the same reaction without base, thiostrepton and DTT were added similarly to above and MeOH (without DIPEA) was added to establish a 1:1 CHCl₃/MeOH. The sample was then analyzed for DTT incorporation by MALDI-TOF MS. Inclusion of a mild base (here, DIPEA, but also DBU or Et₃N or a similar amine) results in more efficient labeling, as expected by the mechanistic nature of the reaction (nucleophilic 1,4-addition).

### Example 7.3

### Screening of Bacterial Extracts for Activated Alkene-Containing Compounds Via Thiol-Based Reactivity Probes

The utility of the labeling was also demonstrated for the same compound in the context of a complex organic extract of its producing organism. For thiostrepton production and labeling, Streptomyces azureus was grown in 10 mL of ISP4 medium (1 L contains 10 g soluble starch, 1 g K₂HPO₄, 1 g MgSO₄, 1 g NaCl, 2 g Na₂SO₄, 2 g CaCO₃, 1 mg FeSO₄, 1 mg ZnSO₄ heptahydrate, 1 mg MnCl₂ heptahydrate) for 7 d at 30° C. Thiostrepton was extracted with 1 mL of CHCl₃ at 23° C. The extract was agitated for 1 min by vortex, submitted to centrifugation (4000×g, 5 min), and the organic layer was removed from the intact, harvested cells. 14 μL of the extract was mixed with DTT (C1) (in MeOH) and DIPEA (in MeOH) to generate a final volume of 20 μL with a final concentration of 500 mM DTT and 10 mM DIPEA, in 7:3 CHCl₃/MeOH, and the mixture was allowed to proceed for 16 h at 23° C. An aliquot (1 μL) of the extract was then mixed with 9 μL of sat. α-cyano-4-hydroxycinnamic acid (CHCA) matrix solution in 1:1 MeCN/H₂O containing 0.1% TFA. 1 μL was spotted onto a steel plate for subsequent MALDI-TOF MS analysis. A representative dehydrated amino acid-containing compound labeled by a thiol-based reactivity probe is shown below.

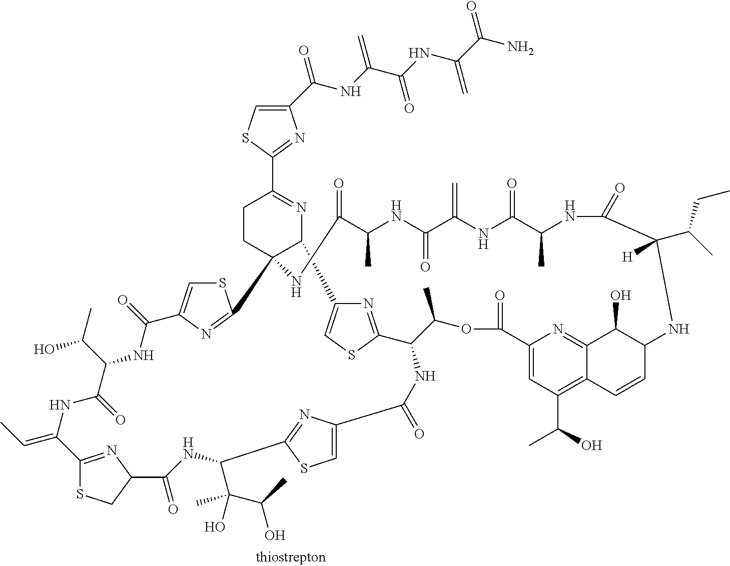 [SMILES: C=C(NC(=O)C(=C)NC(=O)c1csc(C2=N[C@@H]3c4csc(n4)[C@H]4NC(=O)c5csc(n5)[C@H]([C@](C)(O)[C@@H](C)O)NC(=O)C5CSC(=N5)/C(=C/C)NC(=O)[C@H]([C@@H](C)O)NC(=O)c5csc(n5)[C@]3(CC2)NC(=O)[C@H](C)NC(=O)C(=C)NC(=O)[C@H](C)NC(=O)[C@H]([C@@H](C)CC)NC2C=Cc3c([C@H](C)O)cc(nc3[C@H]2O)C(=O)O[C@@H]4C)n1)C(N)=O]

### Example 7.4

Labeling of dehydrated amino acid-containing compounds with thiol probes bearing charged atoms.

Natural products can be labeled with probes containing permanently- or easily-charged moieties in order to enhance detection by mass spectrometry. One such permanently-charged tag is a quaternary amine, as in thiocholine (C2), which was used to label the dehydrated amino acid-containing natural product thiostrepton as an example. A mixture of thiostrepton (0.9 mM), thiocholine chloride C2 (44 mM), and DIPEA (18 mM) in CHCl₃/MeOH/i-PrOH (10:5:2) was allowed to sit at rt overnight. The mixture was then diluted tenfold in MeOH, and 0.7 μL aliquots of this were spotted onto a steel plate with each several different matrices (1 μL each of a saturated solution in 1:1 MeCN/H₂O) before being analyzed by MALDI-TOF MS. Labeling was indicated by the appearance of a peak corresponding to the covalent addition of thiocholine C2 (increase of 120 Da; for thiostrepton [M+thiocholine]⁺=1784 m/z for the addition of a single label).

An easily-charged tag that can be incorporated is a primary amine such as cysteamine (C3). A mixture of thiostrepton (1 mM), cysteamine (C3, varied from 31-500 mM), and DIPEA (10 mM) in CHCl₃/MeOH (ca. 1:1) was allowed to react at rt overnight. The mixture was analyzed by MALDI-TOF MS as above, and successful labeling was indicated by the appearance of peaks corresponding to the addition of cysteamine (for thiostrepton, [M+Na+4 cysteamine]⁺=1994 m/z, consistent with increase of 77 Da per label for 4 labels). Signal enhancement is quantified by comparing ratios of labeled to unlabeled peaks in MALDI-TOF MS compared to the same ratio of peaks by UV-HPLC integrations.

### Example 7.5

### Covalent Capture-and-Release of Thiol-Labeled Molecules Using Disulfide Resins for the Purpose of Affinity Purification

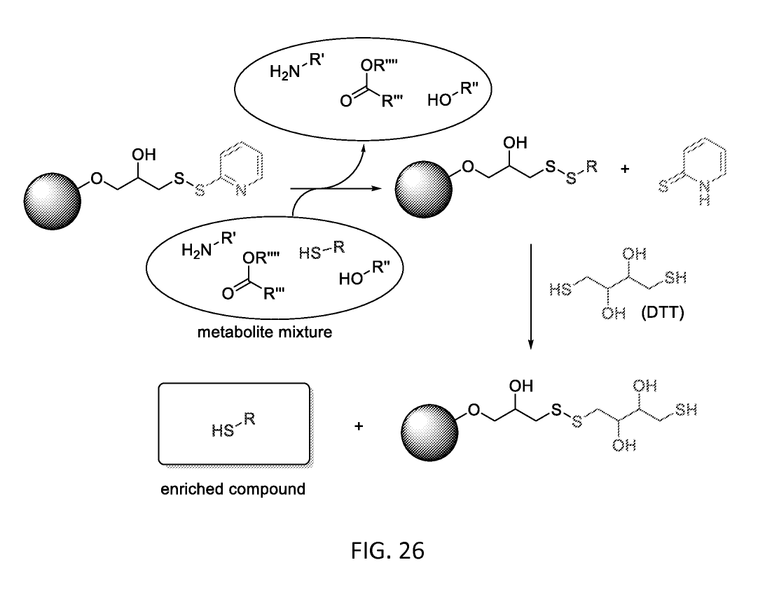

Metabolites covalently labeled with a probe that leaves a pendant thiol group, such as DTT (probe C1), can undergo further covalent tethering to a disulfide-functionalized resin. Non-tethered molecules (those not labeled under the chemistry employed) are not retained on the resin, and after washing, a thiol (such as C1) can be used to elute the bound material via disulfide exchange, allowing the analyte of interest to be enriched (FIG. 26).

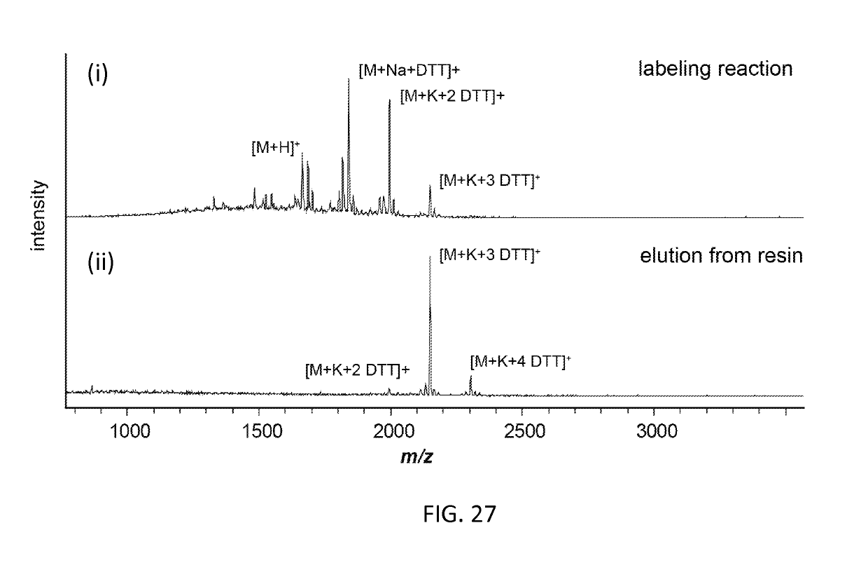

A labeling reaction mixture of a thiostrepton using C1 according to the general procedure described above (conditions: 10 mM thiostrepton, 50 mM DTT, 1 mM DIPEA, 1:2 MeOH/CHCl₃, 16 h, rt, reaction volume 150 μL) was first concentrated. The sample was washed with water (500 μL; aided by vortex mixing) and then suspended in TBS (1 mL; pH 8.0; 0.1 M NaCl, 0.1 M Tris; aided by vortex mixing and sonication; 5% DMSO was added to improve thiostrepton solubility). The sample was centrifuged (17000×g, 3 min) to separate the undissolved material. In a 3 mL syringe body column plugged with glass wool, thiopropyl sepharose 6B resin (100 mg; Amersham Pharmacia Biotech; 71-7105-00) was swelled with water for 15 min before being washed with water (30 mL) and TBS (60 mL). The reaction supernatant was then loaded by gravity onto the column; after passing through, the material was re-loaded onto the column three times before the stopcock was turned off and the elution solution was allowed to incubate with the resin for 1 h. The column was then washed with TBS (30 mL) and eluted with DTT (150 mM) in TBS (10 mL). Fractions were collected and subjected to MALDI-TOF MS analysis after tenfold dilution in 1:1 H₂O/MeCN containing 0.1% formic acid (FA) (see FIG. 27).

Peaks corresponding to the incorporation of 0-3 labels were most prominently seen in the MALDI-TOF mass spectrum of labeled material. Upon subjecting the material to enrichment using the resin above, the elution material primarily showed the presence of a species with 3 labels with additional peaks corresponding to 2 or 4 labels, indicating a combination of enhanced aqueous solubility and preferential binding of multiply-labeled species.

### Example 7.6

### Labeling of Terminal Alkene-Containing Compounds Via Thiol Probes

Natural products containing terminal alkenes can be labeled by a thiol probe at room temperature or upon heating in a solvent that has not been deoxygenated. In many cases, including those described below, the inclusion of a radical thermo initiator or photo initiator is not required for labeling to occur. Labeling is attenuated when oxygen is rigorously excluded from the reaction conditions and is hindered in the presence of mild base but can be accelerated by the addition of acid. Thioethers are formed in an anti-Markovnikov fashion consistent with a radical rather than stepwise ionic mechanism.

The general procedure is as follows. A compound or extract in a nonpolar solvent (typically n-BuOH or CHCl₃) containing a thiol probe compound are either heated or allowed to stand at rt until reaction completion is noted by mass spectrometry or TLC. The solvent is used without degassing under an ambient atmosphere. Analysis is performed by diluting an aliquot of the reaction mixture (1 μL) in MeOH (9 μL) and spotting 1 μL of this onto a steel plate with an equivalent volume of matrix (typically CHCA in 50% aq. MeCN containing 0.1% formic acid) and analyzed by MALDI-TOF MS. Representative terminal alkene-bearing natural products labeled via reaction with thiol probes are shown below.

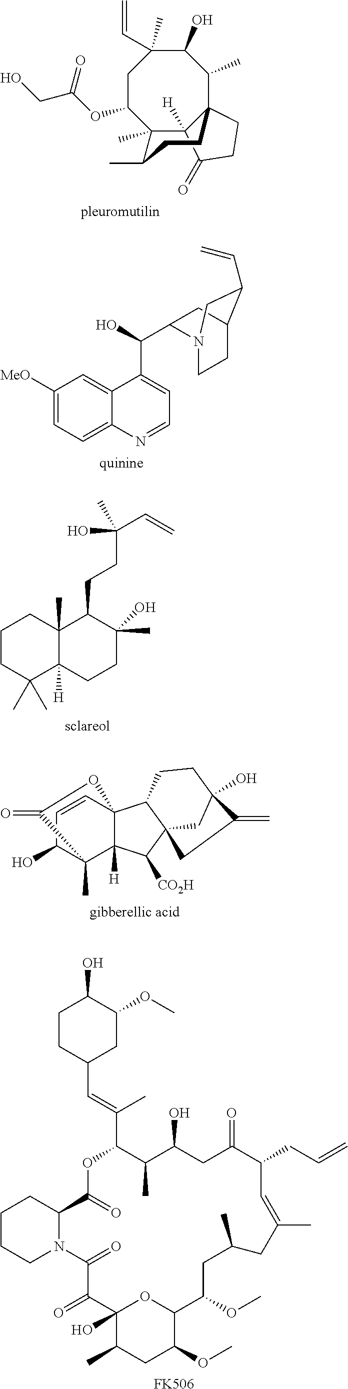

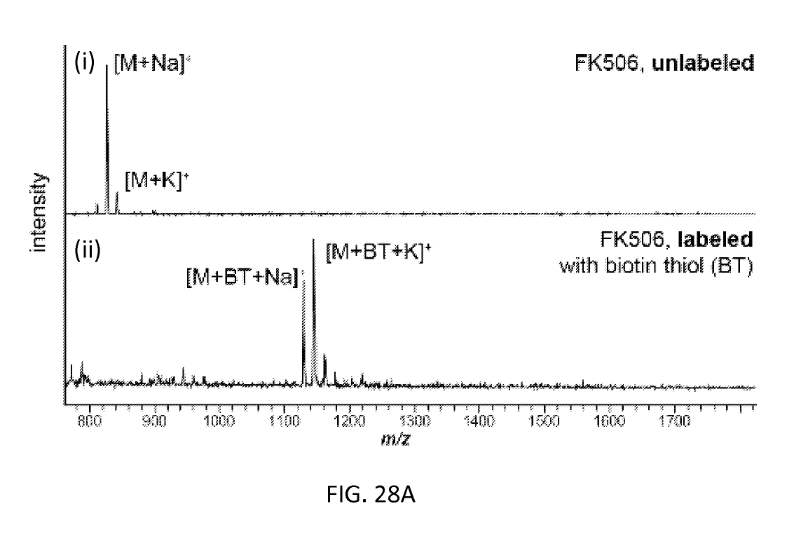

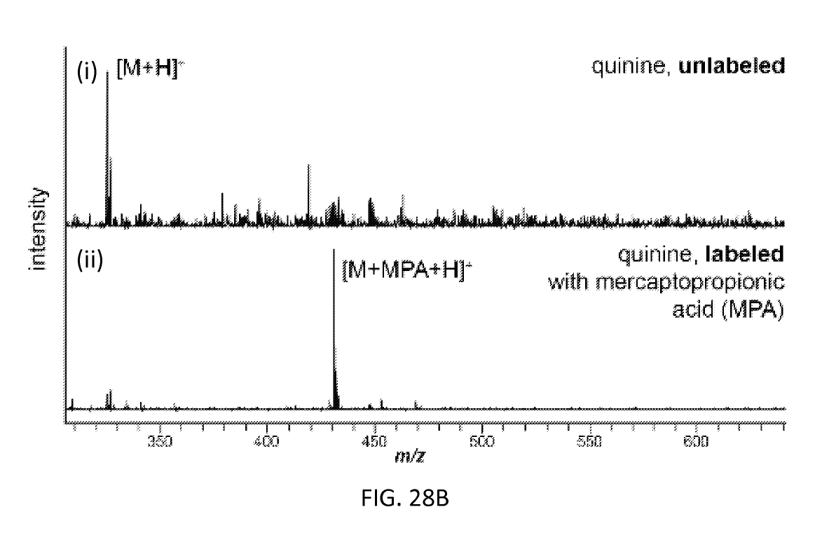

The immunosuppressant FK506 was labeled in this way. Using the general procedure detailed above, a sample of FK506 ([M+Na]⁺ m/z=827) (1 mM) was subjected to labeling with a biotin-linked thiol probe (C6) (100 mM) in n-BuOH at 90° C. for 2 h. A single label was incorporated ([M+BT+Na]⁺ m/z=1130) (see FIG. 28A). Quinine was also labeled in this way. Using the general procedure detailed above, a sample of quinine ([M+H]⁺ m/z=325) (75 mM) was subjected to labeling with 3-mercaptopropionic acid (MPA, probe C7) (0.7 M) in n-BuOH at room temperature overnight. A single label was incorporated ([M+MPA+H]⁺ m/z=431) (FIG. 28B).

### Example 7.7

### Capture-and-Release of Thiol-Labeled Molecules Bearing Biotin Groups Via Affinity Purification

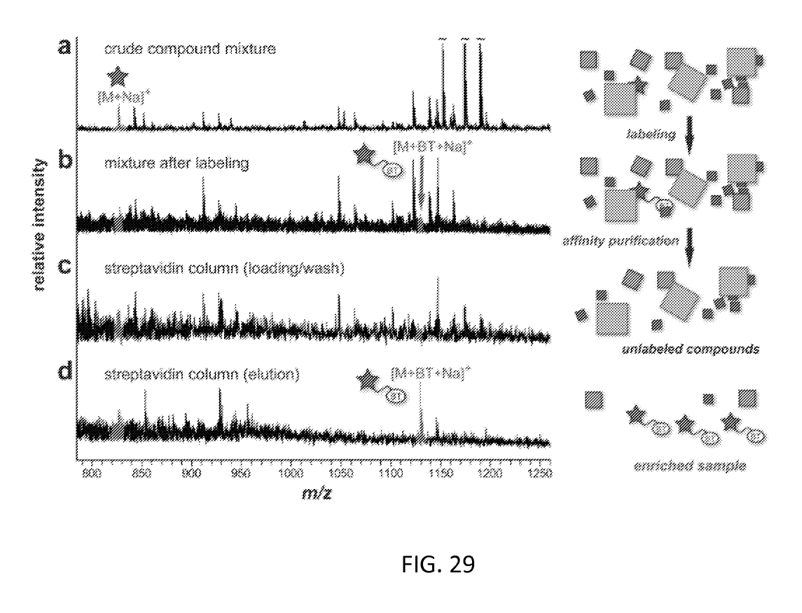

Compounds labeled with biotin-functionalized probes can be enriched by affinity chromatography using a streptavidin resin. Proof of principle was demonstrated by labeling FK506 with a biotinylated thiol probe (C6) in the context of a complex sample. FK506 (2 μM) was added to 1 mL of a saturated MeCN extract of Todd Hewitt broth (BD Biosciences), tryptone (Fisher Scientific), and yeast extract (Fisher Scientific). FK506, initially present only as a minor component of the extract (FIG. 29 (spectrum (a)), was covalently derivatized according to the general procedure previously described (500 μL of extract mixed with 500 μL n-BuOH, to which was added probe C6 [15 mM]; 90° C.; 2 h). The resulting labeled peak [M+BT+Na]⁺ was barely visible in the resulting MALDI-TOF mass spectrum of the reaction mixture in comparison to noise and other peaks (FIG. 29 (spectrum (b)). The reaction mixture was subsequently evaporated, redissolved in water, and subjected to affinity purification with a streptavidin-linked agarose resin (FIG. 29 (spectra (d) and (e)). The peak corresponding to labeled material ([M+BT+Na]⁺, m/z=1130) is significantly enhanced upon elution with MeCN/H₂O (FIG. 29 (spectrum (e)).

### Example 8

Tetrazine-based reactivity probe designs, syntheses and applications.

### Example 8.1

### Tetrazine-Based Reactivity Probe Syntheses

The synthesis of both symmetrically substituted tetrazines (D4-D5) and asymmetrically substituted tetrazines (D1-D3) follows procedures reported in the literature.^{9,5}

The general procedure for synthesis of symmetrical tetrazines is as follows. For the symmetrically substituted tetrazines (D4 and D5), 2 mmol of 2-cyano-3,5-difluoropyridine or 2,3,4,5-tetrafluorobenzonitrile, respectively, were combined with elemental sulfur (0.25 eq.) in EtOH (4 mL). Under an atmosphere of N₂, hydrazine monohydrate (4 eq.) was added drop-wise, and the solution was heated at reflux for 24 h. At this point, the dihydrotetrazine crude product was dissolved in glacial acetic acid (4 mL) and the solution put on ice. To the cooled reaction mixture, sodium nitrite (4 eq.) in H₂O (1 mL) was added drop-wise. Upon addition, the solution turned red, and the cessation of bubbling indicated that the oxidation of the dihydrotetrazine to tetrazine was complete.

The workup for the crude tetrazine product involved extracting the aqueous crude product solution with dichloromethane (DCM) until the organic layer was colorless. The aqueous layer, made basic by the addition of K₂CO₃, was then extracted with DCM again, and the resulting organic fractions were combined. The combined organic fraction was then dried with CaCl₂, filtered, and concentrated by rotary evaporation to give a crude product mixture. The product is purified by standard preparative chromatography methods, such as column chromatography, MPLC, or HPLC, using normal phase (silica) or reversed-phase (C18 silica) stationary phases.

The asymmetric tetrazine (D1-D3) synthesis followed the same procedures as the symmetric tetrazine synthesis, except that acetamidine hydrochloride (5 eq.) was also added with the 2 mmol of 2-cyano-3,5-difluoropyridine or 2,3,4,5-tetrafluorobenzonitrile and sulfur (0.25 eq.). The subsequent portion of the asymmetric tetrazine synthesis and workup are the same.

### 3-(3,5-difluoropyridin-2-yl)-6-methyl-1,2,4,5-tetrazine (D1)

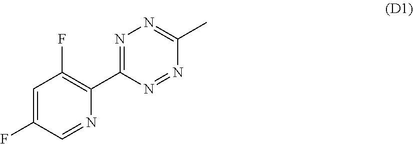

Compound D1 is synthesized according to the general procedure for asymmetrically methyl substituted tetrazines.

### 3-methyl-6-(2,3,4,5-tetrafluorophenyl)-1,2,4,5-tetrazine (D2)

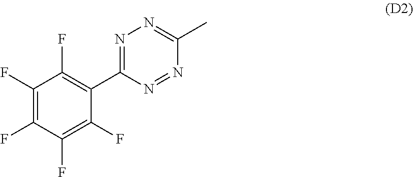 [SMILES: Cc1nnc(-c2c(F)c(F)c(F)c(F)c2F)nn1]

Compound D2 is synthesized according to the general procedure for asymmetrically methyl substituted tetrazines.

### 3-methyl-6-phenyl-1,2,4,5-tetrazine (D3)

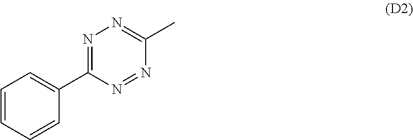

Compound D3 was synthesized according to the general procedure for asymmetrically methyl substituted tetrazines, and the conversion to product was monitored by TLC (1:4 EtOAc/hexane on silica). A 20 g silica column was slurry-loaded, and the crude product mixture of D3 was added and eluted using an isocratic solvent combination of 1:4 ethylacetate:hexane. Compound D3 was the first to elute, and its fractions were collected and concentrated by rotary evaporation.

### 3,6-di-2-(3,5-difluoropyridyl)-1,2,4,5-tetrazine (D4)

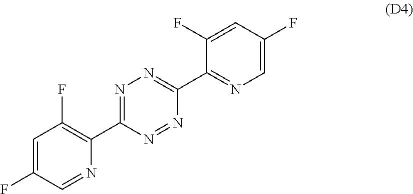 [SMILES: Fc1cnc(-c2nnc(-c3ncc(F)cc3F)nn2)c(F)c1]

Compound D4 was synthesized according to the general procedure for symmetrically substituted tetrazines. The crude product was purified via MPLC (12 g normal phase silica column) using a linear gradient of 1-10% MeOH in DCM. Once an optimized purification on the CombiFlash is found, D4 will be prepped for the final purification on the HPLC.

### 3,6-di-(2,3,4,5-tetrafluorophenyl)-1,2,4,5-tetrazine (D5)

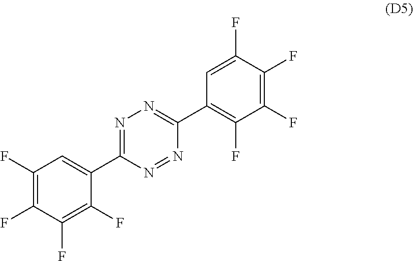 [SMILES: Fc1cc(-c2nnc(-c3cc(F)c(F)c(F)c3F)nn2)c(F)c(F)c1F]

Compound D5 was synthesized according to the general procedure for symmetrically substituted tetrazines. The crude product will be purified on the CombiFlash using a 12 g normal phase silica column with an optimized solvent gradient before purification on the HPLC.

### Example 8.2

### Labeling of Alkene-Containing Compounds Via Tetrazine-Based Reactivity Probes

Compounds containing electron-rich alkene moieties can be covalently labeled by tetrazine-based reactivity probes, forming covalent adducts via a Diels-Alder cyclization, extrusion of N₂, and aromatization as shown in FIG. 2. An exemplary approach for labeling of alkene-containing compounds with tetrazine probes is shown below in Scheme V.

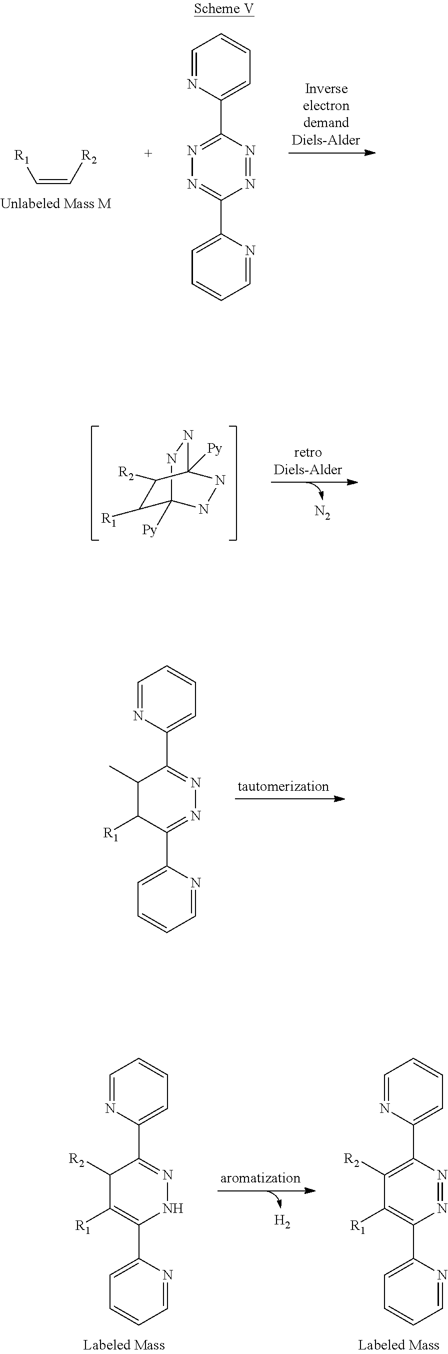

The usefulness of the tetrazine probes was first demonstrated by labeling of representative alkene-bearing natural products, either as crude organic extracts of the corresponding producing microorganisms or as solutions of the purified natural product standards.

Extract labeling reactions were performed in either MeOH or CHCl₃, depending on the solvent of the extract of interest. Labeled compounds exhibit a mass shift of either +206 Da (rearomatized post ligation) or +208 Da (unaromatized) for tetrazine probe D6 (3,6-di-2-pyridyl-1,2,4,5-tetrazine). In labeling reactions performed in CHCl₃, 20 μL of an extract is mixed with 20 μL of 50 mM 3,6-di-2-pyridyl-1,2,4,5-tetrazine (D6) to a final concentration of 25 mM tetrazine in 40 μL solution. In labeling reactions performed in MeOH, 5 μL of an extract is mixed with 20 μL of 10 mM 3,6-di-2-pyridyl-1,2,4,5-tetrazine (D6) to a final concentration of 8 mM tetrazine in 25 μL solution. The extracts were then left to react either at rt or at 50° C. for 16 h. The reacted solution was then analyzed by MALDI-TOF MS after co-spotting an aliquot of the reaction solution (ca. 1 μL) with matrix solution (ca. 1 μL of sat. CHCA in 50% aq. MeCN containing 0.1% formic acid).

For the labeling of purified natural product standards, commercially available compounds were allowed to react and analyzed in a similar manner as above. The choice of reaction solvent depended on the solubility of the natural product being labeled. For reactions performed in CHCl₃, a 40 μL solution was prepared at a final concentration of natural product (1 mM) and 3,6-di-2-pyridyl-1,2,4,5-tetrazine D6 (50 mM). For compounds soluble in MeOH, a 25 μL solution was prepared at a final concentration of natural product (1 mM) and 3,6-di-2-pyridyl-1,2,4,5-tetrazine D6 (8 mM).

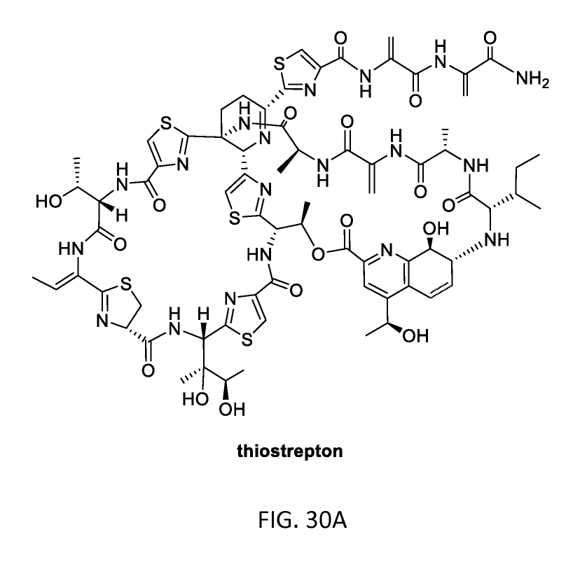

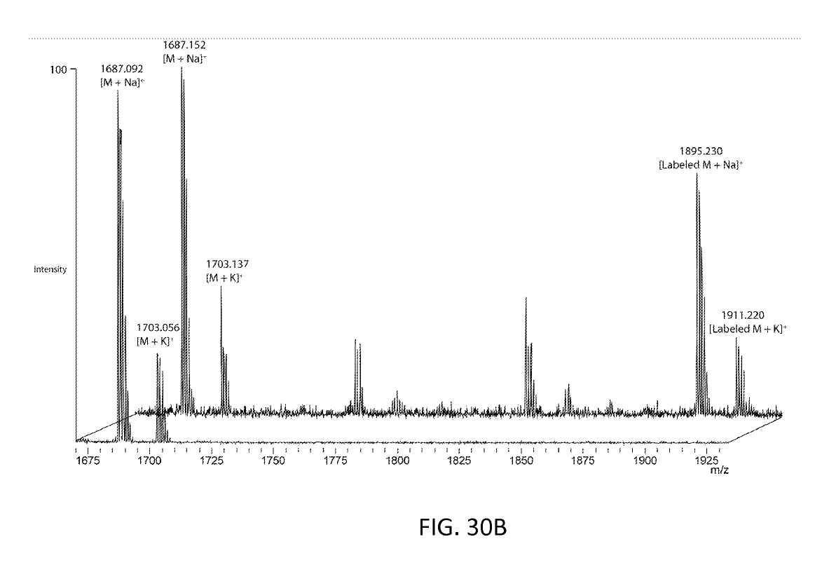

Examples of labeling of purified, representative compounds in solution are given below. Thiostrepton was labeled in 40 μL CHCl₃ at 50° C. (FIG. 30). The front spectrum is thiostrepton (1 mM) alone in 40 μL CHCl₃ heated at 50° C. for 16 h, while the back spectrum is 1 mM thiostrepton with 50 mM 3,6-di-2-pyridyl-1,2,4,5-tetrazine heated at 50° C. for 16 hours. The thiostrepton shows [M+Na]⁺ and [M+K]⁺ at 1687 and 1703 Da, respectively, whereas the labeled thiostrepton shows [M+Na]⁺ and [M+K]⁺ at 1895 and 1911 Da, respectively, a +208 Da difference corresponding to addition of tetrazine D6.

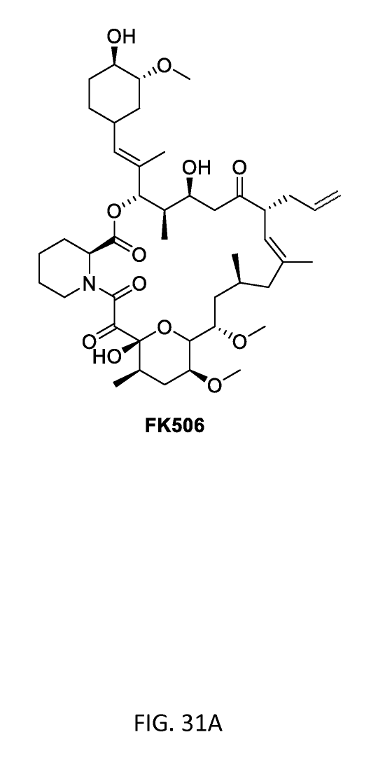

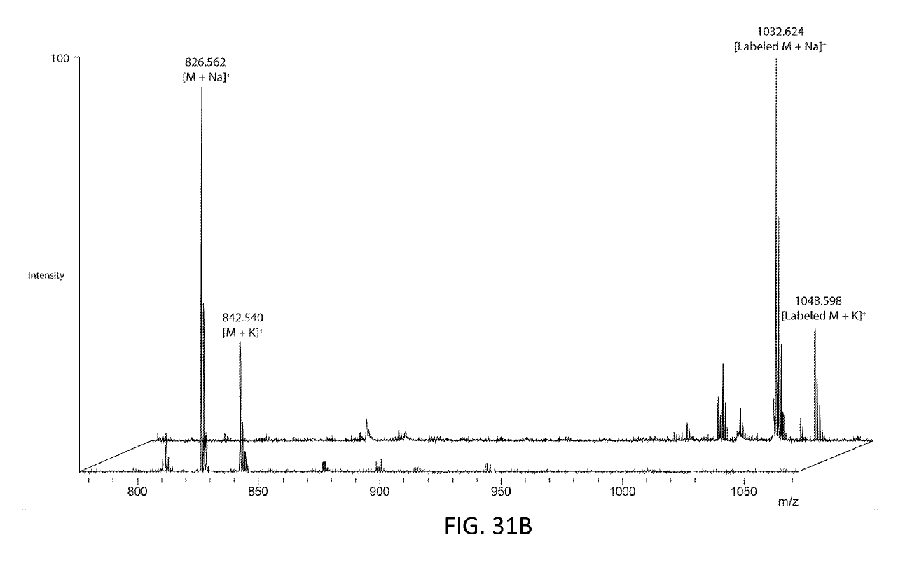

FK506 (tacrolimus) was labeled similarly with probe D6 (FIG. 31). The spectra show the labeling of FK506 under conditions outlined above, at 1 mM FK506 and 50 mM tetrazine D6 in 40 μL CHCl₃ at rt. Almost full conversion, as visualized by MALDI-TOF MS, can be seen for the [M+Na]⁺ peak at 826 Da to the labeled peak at 1032 Da, corresponding to the +206 Da re-aromatized labeled compound.

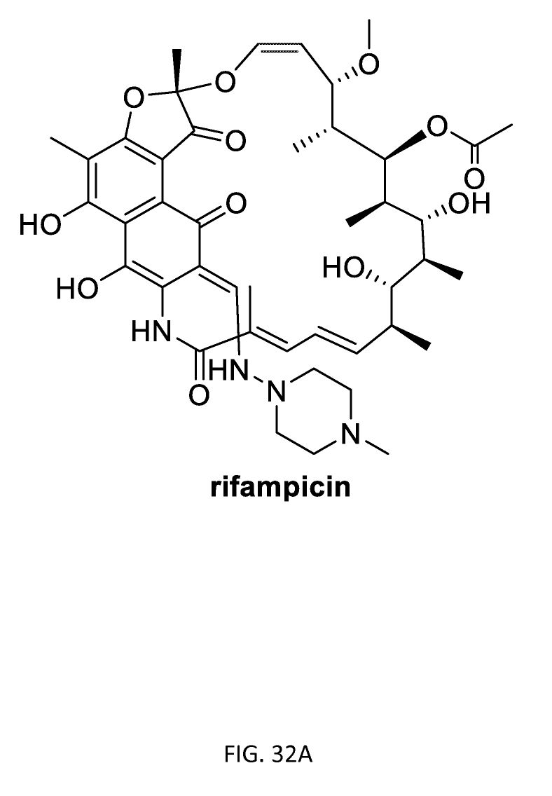

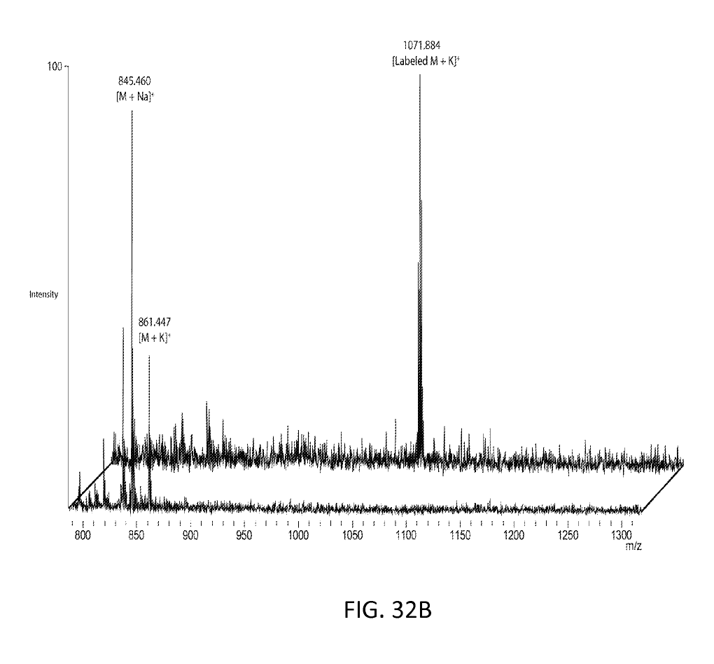

Rifampicin was labeled similarly with probe D6 (FIG. 32). The spectra show rifampicin labeling (1 mM rifampicin, 50 mM tetrazine D6) at 50° C. in CHCl₃, performed according to the general conditions outlined above. The front spectrum shows [M+Na]⁺ and [M+K]⁺ for rifampicin at 845 and 861 Da, respectively, while the back spectrum shows the labeled [M+K]⁺ compound (+210 Da) at 1071 Da.

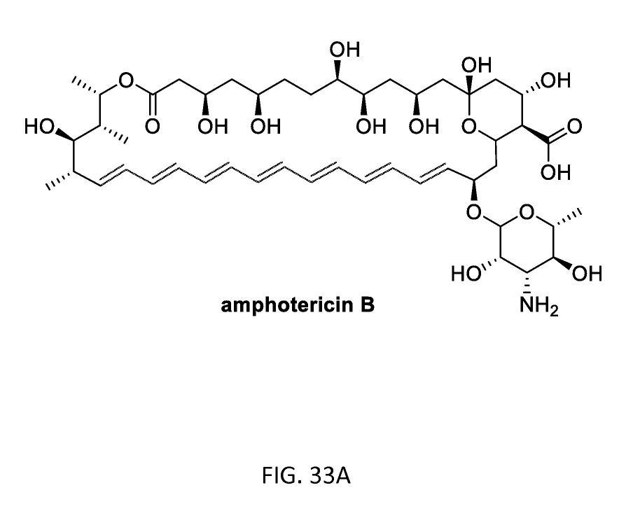

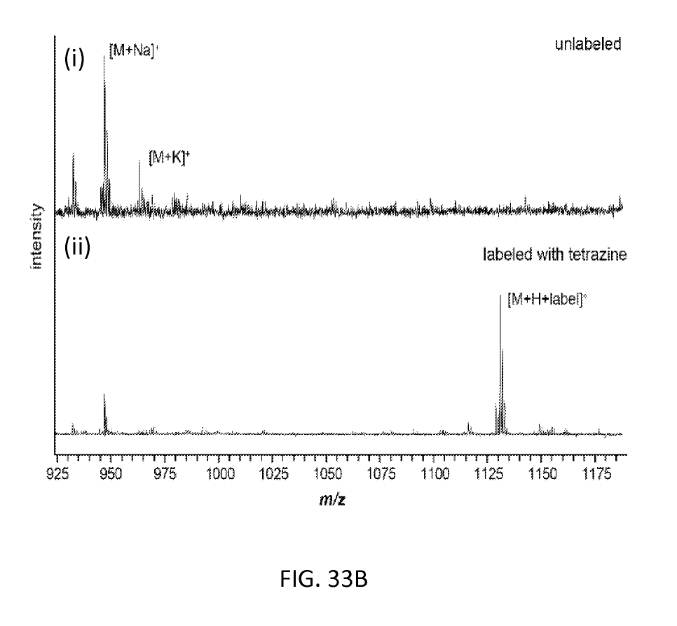

Amphotericin B was labeled with probe D6 using the general conditions outlined previously (FIG. 33). The mass spectra show the labeling of amphotericin B at rt in MeOH (1 mM amphotericin B, 50 mM tetrazine D6). The amphotericin B [M+Na]⁺ peak at 946 Da is converted to the labeled [M+H]⁺ peak of 1130 with +206 Da, corresponding to the addition of tetrazine D6.

### Example 8.3

Labeling of representative compounds in the context of organic extracts of their respective producing microorganisms tetrazine-based probes.

Actinomycete strains were optimized for secondary metabolite production on agar plates of one of the following media: ATCC medium no. 172 (1 L contains 10 g glucose, 20 g soluble starch, 5 g yeast extract, 5 g N-Z Amine, 1 g CaCO₃, 15 g agar); ISP medium no. 4 (1 L contains 10 g soluble starch, 1 g K₂HPO₄, 1 g MgSO₄, 1 g NaCl, 2 g (NH₄)₂SO₄, 2 g CaCO₃, 1 mg FeSO₄.7H₂O, 1 mg MnCl₂.7H₂O, 1 mg ZnSO₄.7H₂O, 15 g agar), altMS medium (1 L contains 10 g mannitol, 10 g soy flour [Wel-Pac brand], 10 g malt extract, 15 g agar); ISP medium no. 2 (1 L contains 4 g yeast extract, 10 g malt extract, 4 g dextrose, 15 g agar); SGG medium (1 L contains 10 g starch, 10 g glucose, 10 g glycerol, 2.5 g corn steep powder, 5 g peptone, 2 g yeast extract, 1 g NaCl, 3 g CaCO₃).

Seed cultures of the Actinomycete strains were grown in 5 mL liquid ATCC medium no. 172 for 3 d at 30° C. before being transferred onto solid media and grown for 7 d at 30° C. After 7 d, the cells were scraped from the surface of the agar, extracted at rt with an optimized solvent (MeOH, CHCl₃, EtOAc, or BuOH), and agitated for 6 h to complete extraction. The extract supernatant was separated from the cell mass by centrifugation (4000×g, 10 min), and any remaining solid was removed by filtration. The extracts were then analyzed by MALDI-TOF MS.

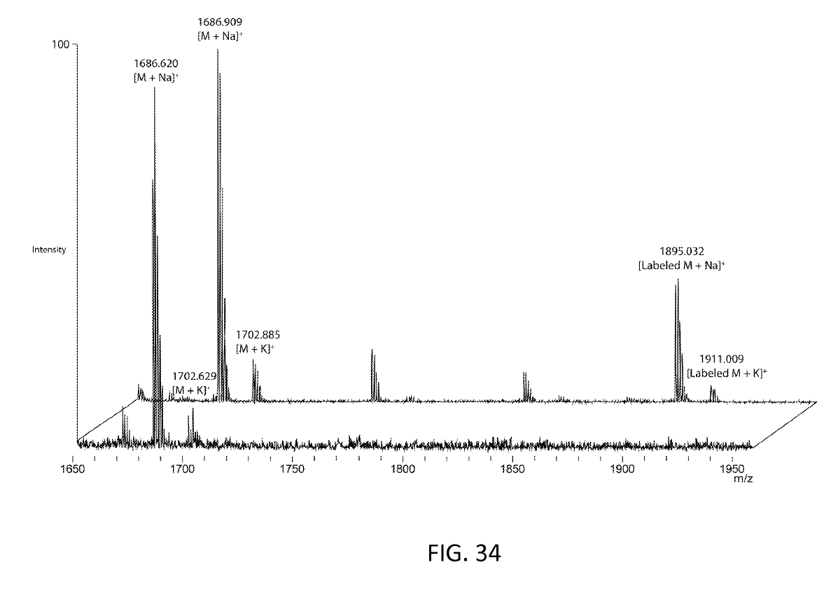

Streptomyces azureus is an actinomycete that produces thiostrepton (FIG. 34). The mass spectrum of a CHCl₃ extract of Streptomyces azureus grown on ISP medium no. 4 for 7 d is shown. The front spectra is the extract alone, with the thiostrepton peak same as shown previously. The back spectra shows labeling with tetrazine D6 (50 mM) under the general conditions outlined above.

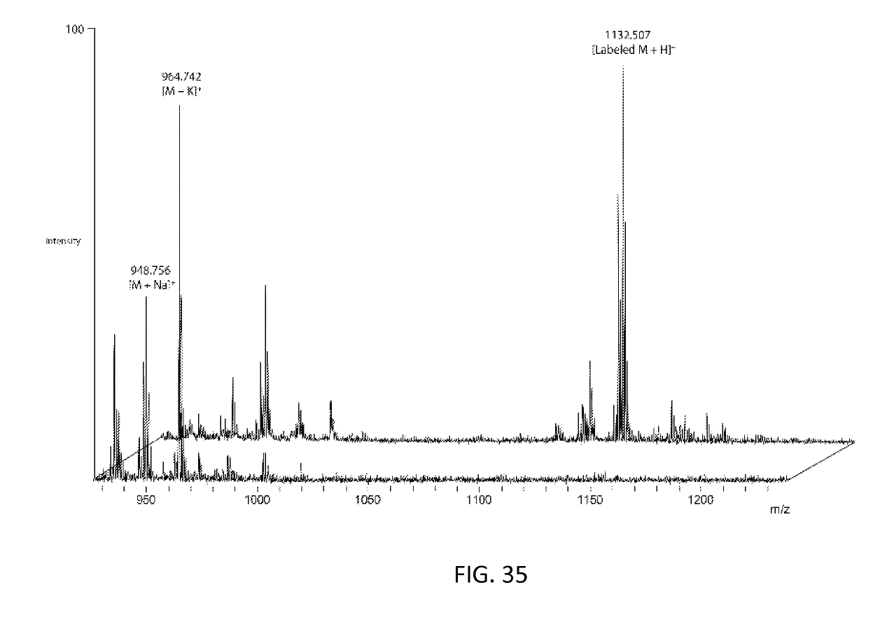

Streptomyces nodosus is an actinomycete which produces amphotericins A and B (FIG. 35). The mass spectra show a MeOH extract of S. nodosus, the producer of amphotericin A and B and the labeling reaction with tetrazine (2.5 mM) at rt for 16 h according to the general conditions described previously. The spectra show the extract amphotericin A peak of [M+Na]⁺ and [M+K]⁺ of 948 and 964 Da respectively, and the D6-labeled [M+H]⁺ peak of 1132 Da.

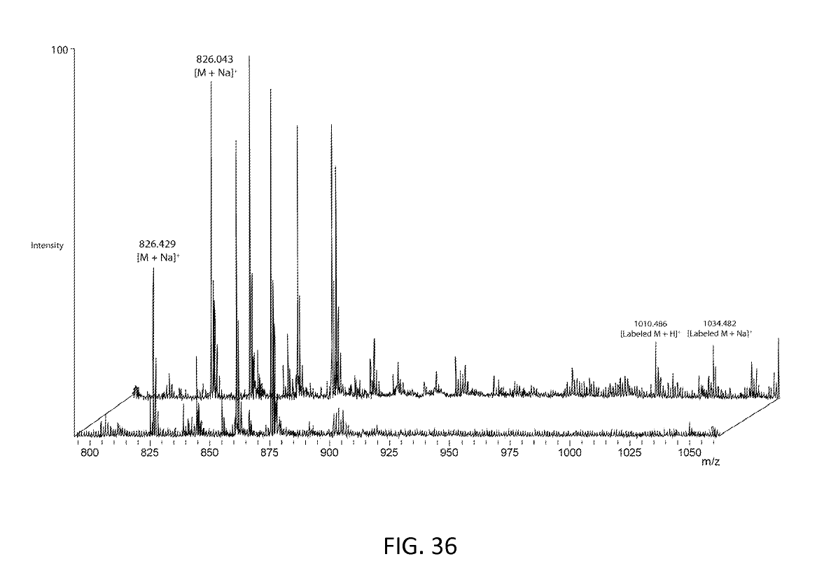

Streptomyces tsukubaensis is an actinomycete which producers FK506 (FIG. 36). The mass spectra show an EtOAc extract of Streptomyces tsukubaensis grown on altMS solid medium for 7 d, as well as a labeling reaction utilizing tetrazine probe D6 in MeOH. There is sparse visible production of FK506, but the back spectrum shows the labeling of the FK506 extract by tetrazine D6 (25 mM) at rt to yield the labeled [M+H]⁺ and [M+Na]⁺ peaks of 1010 and 1034 Da, respectively.

### Example 8.4

Screening of bacterial extracts for alkene-containing compounds via tetrazine-based reactivity probes.

A previously-described collection of extracts of sequenced actinomycetes under differing media conditions were pooled and screened with the tetrazine ligation described above.¹⁰ The conditions for screening were the same as previously mentioned for tetrazine D6 with MeOH extracts. An aliquot of each extract (5 μL) was added to a 20 μL portion of tetrazine D6 in MeOH for a final tetrazine concentration of 8 mM. The reaction was allowed to proceed at rt for 16 h before analysis by MALDI-TOF MS. Multiple hits were found and the hits can be represented by the data below.

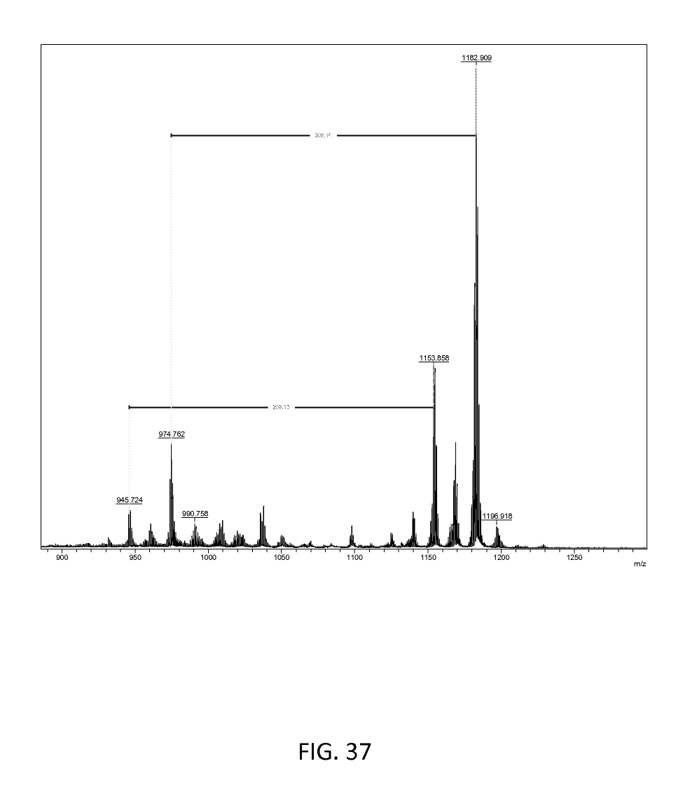

An extract of Streptomyces capuensis NRRL B-12337, upon labeling with probe D6, displayed peaks consistent with labeling (FIG. 37). The unlabeled peaks at 945 and 974 Da show labeled sister peaks at 1153 and 1182 Da respectively, corresponding to a shift of +208 Da, which indicates tetrazine labeling. None of the peaks corresponding to labeling were present in mass spectrum of an unlabeled extract.

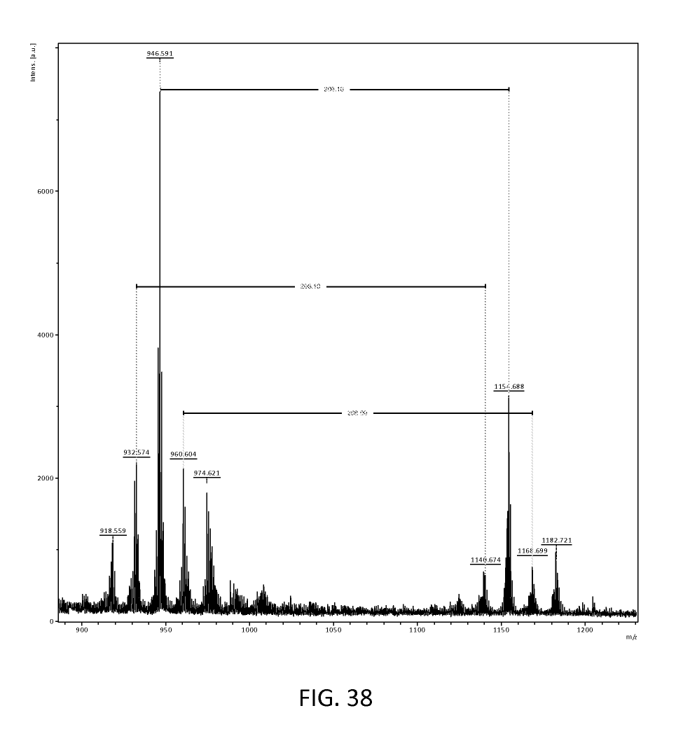

An extract in MeOH of Streptomyces rimosus NRRL WC-3558 labeled with probe D6 under the conditions described above displayed a set of peaks consistent with tetrazine labeling (FIG. 38). There are multiple shifts of +208 Da, indicating tetrazine labeling. Compounds indicated by peaks at 933, 947, 961, and 974 Da were labeled to yield peaks corresponding to 1141, 1155, 1169, and 1183 Da. None of the peaks corresponding to labeling were present in mass spectrum of an unlabeled extract.

Organisms for which extracts display labeling are grown in several media, including ATCC medium no. 172, ISP medium no. 4 and altMS medium (all described previously) to optimize production conditions for their respective screening hits. The media conditions corresponding to the highest MS signal from the compound are scaled up, and compounds are isolated by standard extraction and chromatography techniques (SPE, MPLC, HPLC) for structural characterization by NMR, UV-Vis, and HR-MS, as well as testing of biological activity.

All references, including publications, patent applications, and patents, cited herein are hereby incorporated by reference to the same extent as if each reference were individually and specifically indicated to be incorporated by reference and were set forth in its entirety herein.

Also incorporated by reference in their entirety are any polynucleotide and polypeptide sequences which reference an accession number correlating to an entry in a public database, such as those maintained by The Institute for Genomic Research (TIGR) on the world wide web at tigr.org and/or the National Center for Biotechnology Information (NCBI) on the world wide web at ncbi.nlm.nih.gov.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of identifying a natural product comprising NP—[X]ₙ, the method comprises:
selecting an organism having a biosynthetic pathway for producing the natural product comprising NP—[X]ₙ using a bioinformatics algorithm;
preparing a sample suspected to contain NP—[X]ₙ comprising a complex cellular metabolite mixture from the organism;
reacting the sample suspected to contain NP—[X]ₙ with reactivity probe Y according to Scheme I:
NP—[X]ₙ+Y→NP—[X]ₙ₋ₘ[Z]ₘ Scheme I,
wherein NP—[X]ₙ represents a natural product NP having a chemical moiety X that is susceptible to chemical modification by reactivity probe Y to form at least one product adduct NP—[X]ₙ₋ₘ[Z]ₘ, in which chemical moiety X reacts with reactivity probe Y to form adduct Z, wherein n ranges from 1 to about 10 and m is at least 1 and m≦n;
optionally dereplicating the product collection of at least one known labeled metabolite to provide a depleted product collection comprising at least one unknown labeled metabolite; and
determining the structure of at least one unknown labeled metabolite, thereby identifying the natural product comprising NP—[X]ₙ.

2. The method of claim 1, wherein the bioinformatics algorithm comprises:
populating a list of strains encoding a first biosynthetic enzyme;
reducing the list of strains encoding a second biosynthetic enzyme to yield a refined list of strains, wherein the second biosynthetic enzyme is encoded by a gene within a range of ten open reading frames of a gene encoding the first biosynthetic enzyme; and
identifying precursor peptide products of the first biosynthetic enzyme from the refined list of strains,
wherein both the first and second biosynthetic enzymes catalyze transformations in the biosynthetic pathway for producing the natural product comprising NP—[X]ₙ.

3. The method of claim 2, wherein the first biosynthetic enzyme comprises a thiazole/oxazole-modified microcin (TOMM) cyclodehydratase and the second biosynthetic enzyme comprises a lantibiotic dehydratase, and chemical moiety X is a dehydrated amino acid.

4. The method of claim 1, further comprising the step of dereplicating the product collection of at least one known labeled metabolite to provide a depleted product collection comprising at least one unknown labeled metabolite.

5. The method of claim 4, wherein the step of dereplicating the product collection of at least one known labeled metabolite comprises:
identifying the presence in the product collection comprising labeled metabolites the at least one known labeled metabolite having a mass of a labeled natural product predicted from a precursor peptide product from the organism selected using the bioinformatics algorithm; and
removing the at least one known labeled metabolite from further characterization.

6. The method of claim 5, wherein the step of identifying the presence in the product collection comprising labeled metabolites the at least one known labeled metabolite comprises applying differential mass spectrometry to characterize the at least one known labeled metabolite.

7. The method of claim 4, wherein the step of dereplicating the product collection of at least one known labeled metabolite comprises applying differential mass spectrometry to characterize the product collection.

8. The method of claim 1, wherein the organism is a bacterium or a fungus.

9. The method of claim 1, wherein reactivity probe Y has the structure of Formula (I):
R-L-Q (I),
wherein R is a reactive moiety that reacts with chemical moiety X, L is a linker and Q is a label.

10. The method of claim 9, wherein label Q is selected from an affinity label, a detectable group and a physicochemical label.

11. The method of claim 9, wherein label Q comprises an affinity probe.

12. The method of claim 11, wherein the affinity probe is selected from biotin, streptavidin, polyhistine, an unreacted thiol group of dithiothreitol, glutathione-S-transferase (GST), HaloTag®, AviTag, Calmodulin-tag, polyglutamate tag, FLAG-tag, HA-tag, Myc-tag, S-tag, SBP-tag, Softag 3, V5 tag, Xpress tag, and a hapten.

13. The method of claim 11, wherein the affinity probe comprises Formula (A):

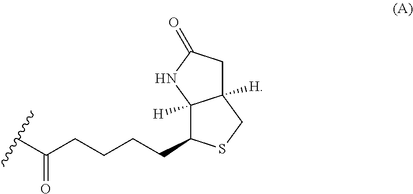

14. The method of claim 9, wherein label Q comprises a detectable group.

15. The method of claim 14, wherein the detectable group is selected from a radiolabel, a fluorescent label, and a chemiluminescent label.

16. The method of claim 14, wherein the detectable group comprises a fluorescent label.

17. The method of claim 16, wherein the fluorescent label comprises Formula (B):

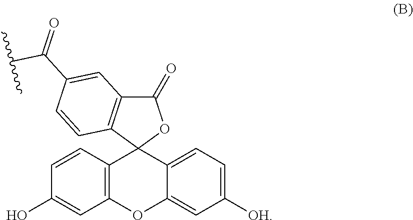 [SMILES: *C(=O)c1ccc2c(c1)C(=O)OC21c2ccc(O)cc2Oc2cc(O)ccc21]

18. The method of claim 9, wherein label Q comprises a physicochemical label.

19. The method of claim 18, wherein the physicochemical label is selected from an isotopic label and a mass label.

20. The method of claim 18, wherein the physicochemical label comprises a cation mass label.

21. The method of claim 20, wherein the cation mass label comprises Formula (C):

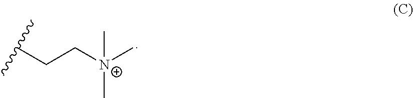 [SMILES: *CC[N+](C)(C)C]

22. The method of claim 9, wherein label Q is selected from the following: 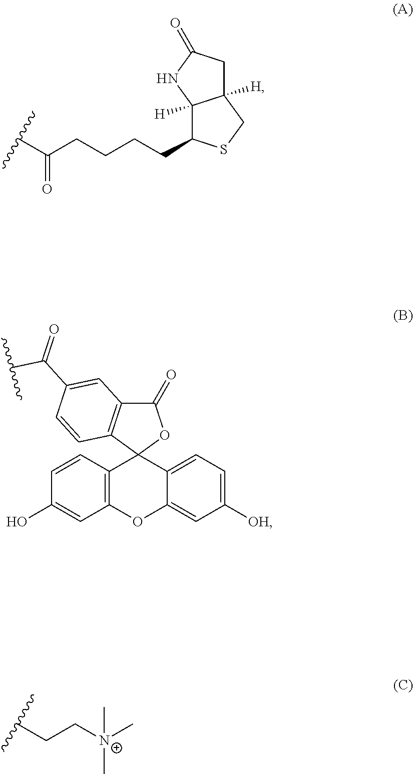
and combinations thereof.

23. The method of claim 1, wherein reactivity probe Y is selected from the following: 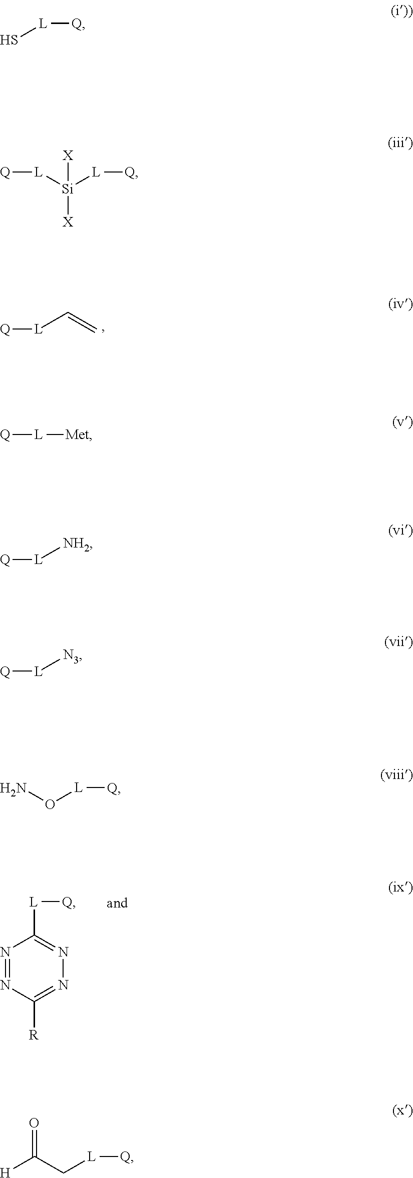
or a combination thereof,
wherein R is alkyl or L-Q.

24. The method of claim 1, wherein reactivity probe Y is selected from an aminooxy-based reactivity probe, an aldehyde-based reactivity probe, a thiol-based reactivity probe and a tetrazine-based reactivity probe, or a combination thereof.

25. The method of claim 1, wherein reactivity probe Y comprises an aminooxy-based reactivity probe.

26. The method of claim 25, wherein the aminooxy-based reactivity probe is selected from 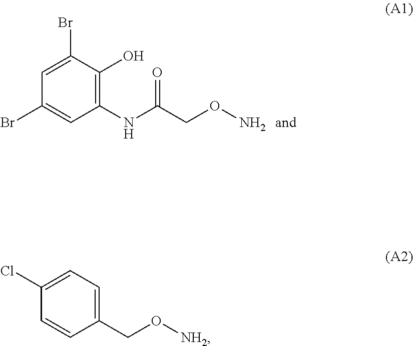 [SMILES: NOCC(=O)Nc1cc(Br)cc(Br)c1O ; NOCc1ccc(Cl)cc1]
or a combination thereof.

27. The method of claim 1, wherein reactivity probe Y comprises an aldehyde-based reactivity probe.

28. The method of claim 27, wherein the aldehyde-based reactivity probe is

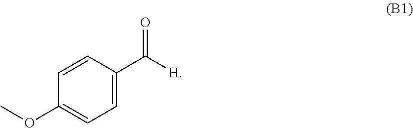

29. The method of claim 1, wherein reactivity probe Y comprises a thiol-based reactivity probe.

30. The method of claim 29, wherein the thiol-based reactivity probe is selected from 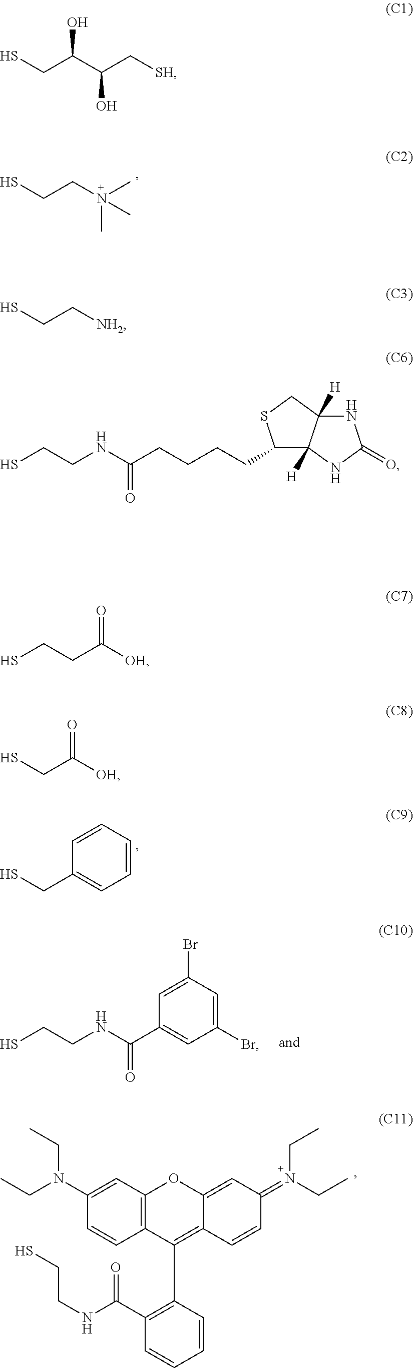
or a combination thereof.

31. The method of claim 1, wherein reactivity probe Y comprises a tetrazine-based reactivity probe.

32. The method of claim 31, wherein the tetrazine-based reactivity probe is selected from 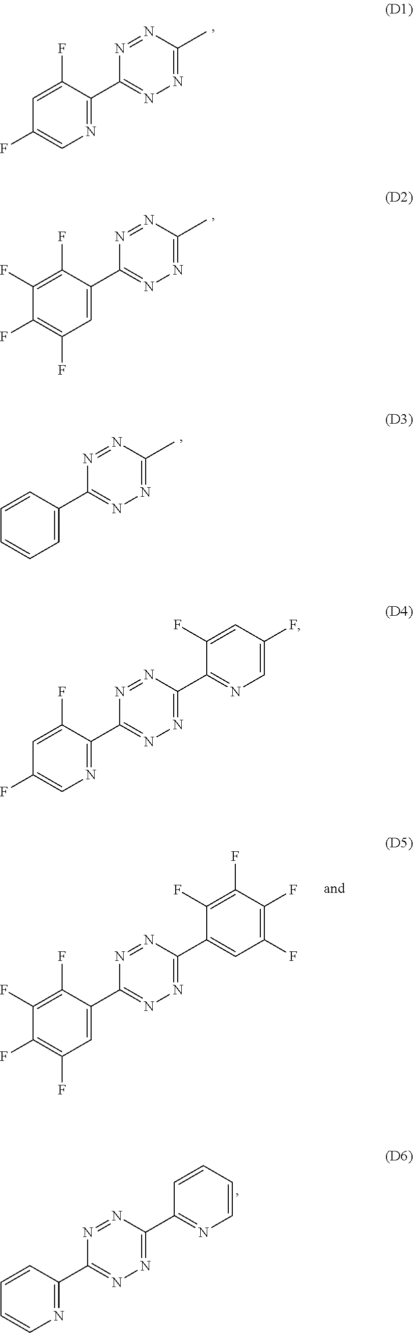 [SMILES: Cc1nnc(-c2ncc(F)cc2F)nn1 ; Cc1nnc(-c2cc(F)c(F)c(F)c2F)nn1 ; Cc1nnc(-c2ccccc2)nn1 ; Fc1cnc(-c2nnc(-c3ncc(F)cc3F)nn2)c(F)c1 ; Fc1cc(-c2nnc(-c3cc(F)c(F)c(F)c3F)nn2)c(F)c(F)c1F ; c1ccc(-c2nnc(-c3ccccn3)nn2)nc1]
or a combination thereof.

33. The method of claim 1, wherein the step of determining the structure of the at least one unknown labeled metabolite comprises at least one selected from the group consisting of mass spectrometry, UV-VIS spectroscopy, nucleic resonance spectrometry and infrared spectroscopy or combinations thereof.

34. A natural product comprising NP—[X]ₙ identified with the method of claim 1.

35-69. (canceled)
